(19) United States
(12) Patent Application Publication
(10) Pub. No.: US 2026/0190857 A1
(43) Pub. Date: Jul. 2, 2026

(54) ORGANIC COMPOUND AND LIGHT-EMITTING DEVICE

(71) Applicant: SEMICONDUCTO ENERGY LABORATORY CO., LTD., Atsugi-shi (JP)

(72) Inventors: Keito TOSU, Atsugi (JP); Takeyoshi Watabe, Atsugi (JP); Yui Yoshiyasu, Atsugi (JP); Tomohiro Kubota, Atsugi (JP); Nobuharu Ohsawa, Zama (JP); Harue Osaka, Atsugi (JP)

(21) Appl. No.: 19/431,760

(22) Filed: Dec. 23, 2025

(30) Foreign Application Priority Data
Dec. 26, 2024 (JP) 2024-230213

(51) Int. Cl. H10K 85/60; C07D 403/10; C07F 5/04; C07F 7/08; H10K 50/16; H10K 85/40

(52) U.S. Cl. CPC H10K 85/654; C07D 403/10; C07F 5/04; C07F 7/0812; H10K 85/40; H10K 50/16

(57) ABSTRACT

An organic compound with a low refractive index is provided. An organic compound represented by General Formula (G1) is provided. In General Formula (G1), each of Q¹ to Q³ independently represents N or CH; at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen, an alkyl group having 1 to 6 carbon atoms, or a cycloalkyl group having 3 to 10 carbon atoms; each of R¹¹ to R²⁴ independently represents hydrogen, an alkyl group having 1 to 6 carbon atoms, a cycloalkyl group having 3 to 10 carbon atoms, a substituted or unsubstituted aryl group having 6 to 30 carbon atoms, or a substituted or unsubstituted heteroaryl group having 3 to 30 carbon atoms; and at least any one of R¹¹ to R²⁴ represents a substituted or unsubstituted heteroaryl group having 3 to 30 carbon atoms.

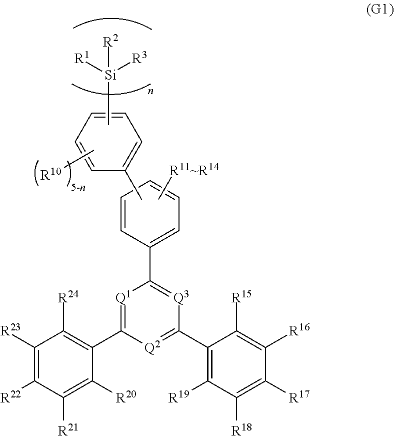

### BACKGROUND OF THE INVENTION

### 1. Field of the Invention

One embodiment of the present invention relates to an organic compound, an organic semiconductor device, a light-emitting device, a light-receiving device, a light-emitting apparatus, a light-receiving apparatus, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor apparatus, a display device, a liquid crystal display apparatus, a light-emitting apparatus, a lighting device, a power storage device, a memory device, an imaging device, a driving method thereof, and a manufacturing method thereof.

### 2. Description of the Related Art

Light-emitting devices (organic EL devices) that include organic compounds and utilize electroluminescence (EL) have been put to practical use. In the basic structure of such light-emitting devices, an organic compound layer (EL layer) including a light-emitting material is sandwiched between a pair of electrodes. Carriers are injected by application of voltage to the device, and recombination energy of the carriers is used, whereby light emission can be obtained from the light-emitting material.

Such light-emitting devices are of self-luminous type and thus have advantages over liquid crystal devices such as high visibility and no need for backlight when used in pixels of a display, and are suitable as devices used in flat panel displays. Displays that include such light-emitting devices are also highly advantageous in that they can be thin and lightweight. Another feature of such organic EL elements is that they have an extremely fast response speed. Since a continuous planar light-emitting layer can be formed for such light-emitting devices, planar light emission can be achieved. This feature is difficult to realize with point light sources typified by incandescent lamps or LEDs or linear light sources typified by fluorescent lamps; thus, such light-emitting devices also have great potential as planar light sources, which can be applied to lighting devices and the like.

Displays or lighting devices including light-emitting devices can be used for a variety of electronic appliances as described above, and research and development of light-emitting devices has progressed for more favorable characteristics.

Low outcoupling efficiency is often a problem in an organic EL device. In particular, the attenuation due to reflection which is caused by a difference in refractive index between adjacent layers is a main cause of a reduction in device efficiency. In order to reduce this effect, a structure including a layer formed using a low refractive index material in an EL layer (see Non-Patent Document 1, for example) has been proposed. A light-emitting device having this structure can have higher outcoupling efficiency and higher external quantum efficiency than a light-emitting device having a conventional structure.

The EL layer of an organic EL element can be formed by any of a variety of methods such as a vacuum evaporation method, a gravure printing method, an offset printing method, a screen printing method, an inkjet method, and a spin coating method. According to recent findings, giant surface potential (GSP) is sometimes formed on a film obtained by a vacuum evaporation method. The term GSP refers to a phenomenon due to a spontaneous orientation polarization (SOP) caused by deviation in the thickness direction of permanent electric dipole moment orientation of an evaporated film of an organic compound.

The surface potential of an evaporated film with GSP changes linearly with increasing thickness without saturation. For example, the surface potential of an evaporated film of tris(8-quinolinolato)aluminum(III) (abbreviation: Alq₃) reaches approximately 28 V at a thickness of 560 nm. The electric field strength reaches 5×10⁵ V/cm, which is approximately the same level as electric field strength during driving of a general light-emitting device.

The slope of a GSP (GSP slope) is represented by ΔV/Δd, where ΔV is the amount of change in surface potential (mV) and Ad is the amount of change in the thickness (nm) of a film whose GSP changes in proportion to the thickness. Note that a GSP slope of a film whose surface potential increases with increasing thickness is a positive GSP slope, and a GSP slope of a film whose surface potential decreases with increasing thickness is a negative GSP slope. It can be said that Alq₃ described above is a material that has a positive GSP slope in the form of a film. The potential of a layer with a positive GSP slope is lower on the substrate side, and the potential of a layer with a negative GSP slope is higher on the substrate side.

As described above, GSP is a phenomenon due to SOP caused by deviation in the thickness direction of permanent electric dipole moment orientation. That is, the following phenomena can be regarded as occurring: in a layer with a positive GSP slope, negative polarization charge is induced on the side where evaporation starts (the substrate side) and positive polarization charge is induced on the side where evaporation ends (the second electrode side); in a similar manner, in a layer with a negative GSP_slope, positive polarization charge is induced on the side where evaporation starts (the substrate side) and negative polarization charge is induced on the side where evaporation ends (the second electrode side). Thus, GSP originates in such induction of polarization charge.

Evaporated films of most organic compounds have a positive GSP slope; thus, in the case where a first layer is deposited on and in contact with a second layer, for example, a GSP slope of the first layer and a GSP slope of the second layer are denoted by the same positive sign. In this case, a polarization charge of the second layer on the first layer side is canceled out by a polarization charge of the first layer on the second layer side, and only a remaining charge can be regarded as an interface charge (fixed charge) at the interface between the first layer and the second layer.

Such interface charge at the interface between evaporated films might cause adverse effects on the characteristics of an organic EL element. Therefore, research and development has been conducted to control the GSP of an evaporated film of an organic compound. For example, Non-Patent Document 2 discloses that the GSP slope of an evaporated film significantly changes depending on the substituent introduced into the organic compound.

### REFERENCES

[Non-Patent Document 1] Jaeho Lee et al., “Synergetic electrode architecture for efficient graphene-based flexible organic light-emitting diodes”, Nature COMMUNICATIONS, Jun. 2, 2016, DOI: 10.1038/ncomms 11791.
[Non-Patent Document 2] Masaki Tanaka et al., “Spontaneous formation of metastable orientation with well-organized permanent dipole moment in organic glassy films”, Nature Materials, 2022, Vol. 21, pp. 819-825.
[Non-Patent Document 3] Yutaka Noguchi et al., “Charge accumulation at organic semiconductor interfaces due to a permanent dipole moment and its orientational order in bilayer devices”, Journal of Applied Physics, 2012, 111, 114508.
[Non-Patent Document 4]Y. Noguchi et al., “Spontaneous Orientation Polarization of Polar Molecules and Interface Properties of Organic Electronic Devices”, Journal of the Vacuum Society of Japan, 2015, Vol. 58, No. 3.

### SUMMARY OF THE INVENTION

In improving the characteristics of an organic EL element, it is useful to use an organic compound with a high carrier-transport property, a low refractive index, and a small GSP slope in a carrier-transport layer of the organic EL element. However, such an organic compound has not been easy to develop.

That is because a low refractive index has a trade-off relationship with a high carrier-transport property or high reliability in a light-emitting device. This problem arises because the carrier-transport property, reliability, and the like of an organic compound are largely dependent on the presence of an unsaturated bond, and an organic compound having many unsaturated bonds tends to have a high refractive index. When an organic compound has many saturated hydrocarbon groups to have a low refractive index, it tends to suffer from a poor carrier-transport property, low reliability, or the like.

Saturated hydrocarbon groups, which do not have conjugation, cause smaller intermolecular dispersion force and van der Waals forces than unsaturated hydrocarbon groups. Because of this, when a vacuum-evaporated film is formed using a compound having saturated and unsaturated hydrocarbon groups, the molecules tend to orient so that the unsaturated hydrocarbon group sites face the substrate or already deposited film and the saturated hydrocarbon group sites face the film surface. Additionally, since a saturated hydrocarbon group is an electron-donating group, the dipole moment of the molecule tends to be such that the saturated hydrocarbon group side is positively polar. Thus, due to the relationship between the orientation generated by a vacuum evaporation method and the permanent electric dipole moment of the molecule, a film formed from a compound having a saturated hydrocarbon group tends to exhibit a large positive GSP slope.

In view of the above, an object of one embodiment of the present invention is to provide an organic compound that has a low reflective index in the form of a film. Another object is to provide an organic compound that has a small GSP slope in the form of a film. Another object is to provide an organic compound that has a carrier-transport property. Another object is to provide an organic compound that has a high carrier-transport property, a low refractive index, and a small GSP slope in the form of a film. Another object is to provide a novel organic compound.

Another object of one embodiment of the present invention is to provide a light-emitting device having high emission efficiency. Another object is to provide a light-emitting device having a low driving voltage. Another object is to provide a light-emitting device, a light-emitting apparatus, an electronic appliance, a display device, and an electronic device each having low power consumption.

Note that the description of these objects does not preclude the existence of other objects. One embodiment of the present invention does not need to achieve all of these objects. Other objects will be apparent from and can be derived from the description of the specification, the drawings, the claims, and the like.

One embodiment of the present invention is an organic compound represented by General Formula (G1).

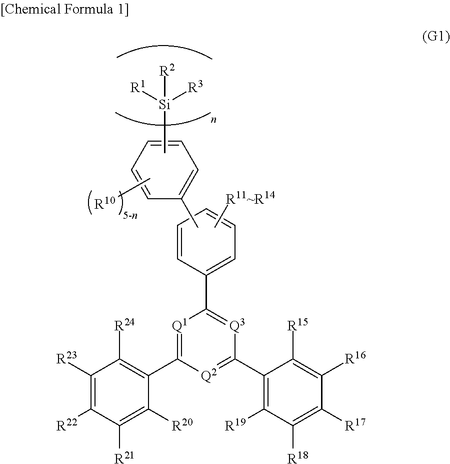

In General Formula (G1), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹¹ to R²⁴ independently represents hydrogen (including deuterium), an alkyl group having 1 to 6 carbon atoms, a cycloalkyl group having 3 to 10 carbon atoms, a substituted or unsubstituted aryl group having 6 to 30 carbon atoms, or a substituted or unsubstituted heteroaryl group having 3 to 30 carbon atoms; at least any one of R¹¹ to R²⁴ represents a substituted or unsubstituted heteroaryl group having 3 to 30 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

Another embodiment of the present invention is an organic compound represented by General Formula (G2).

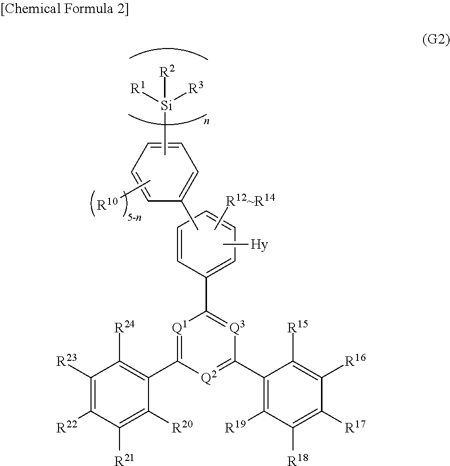

In General Formula (G2), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; Hy represents a substituted or unsubstituted heteroaryl group having 3 to 30 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

In the organic compound having any of the above structures, the substituted or unsubstituted heteroaryl group having 3 to 30 carbon atoms preferably includes nitrogen.

In the organic compound having any of the above structures, one or more of atoms included in an aromatic ring of the substituted or unsubstituted heteroaryl group having 3 to 30 carbon atoms are preferably nitrogen atoms.

In the above organic compound, the substituted or unsubstituted heteroaryl group having 3 to 30 carbon atoms is preferably a substituted or unsubstituted pyridinyl group, a substituted or unsubstituted pyrimidinyl group, or a substituted or unsubstituted pyrazinyl group.

In the organic compound having any of the above structures, the substituted or unsubstituted heteroaryl group having 3 to 30 carbon atoms preferably includes at least any one of an alkyl group having 1 to 6 carbon atoms and a cycloalkyl group having 3 to 10 carbon atoms.

Another embodiment of the present invention is an organic compound represented by General Formula (G3).

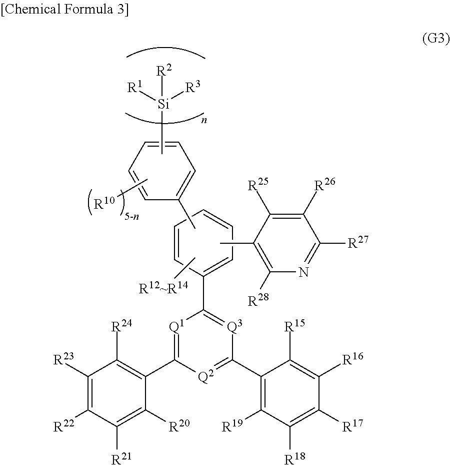

In General Formula (G3), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; each of R²⁵ to R²⁸ independently represents hydrogen (including deuterium), an alkyl group having 1 to 6 carbon atoms, or a cycloalkyl group having 3 to 10 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

Another embodiment of the present invention is an organic compound represented by General Formula (G4).

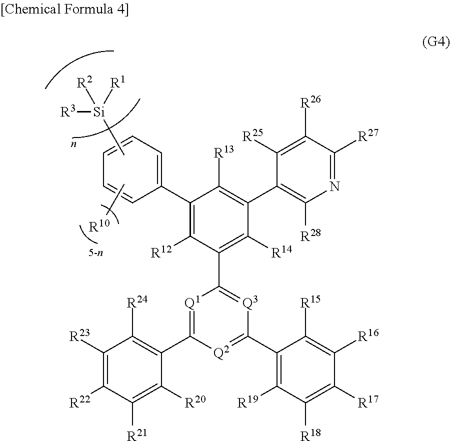

In General Formula (G4), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; each of R²⁵ to R²⁸ independently represents hydrogen (including deuterium), an alkyl group having 1 to 6 carbon atoms, or a cycloalkyl group having 3 to 10 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

In the organic compound having any of the above structures, n is preferably 2 or 3, further preferably 2.

Another embodiment of the present invention is an organic compound represented by General Formula (G5).

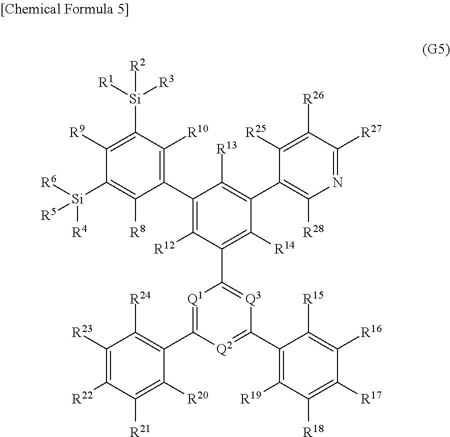

In General Formula (G5), each of Q¹ to Q³ independently represents N or CH (including CD); at least two of Q¹ to Q³ represent N; each of R¹ to R⁶ independently represents an alkyl group having 1 to 6 carbon atoms or a phenyl group; each of R⁸ to R¹⁰ independently represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; and each of R²⁵ to R²⁸ independently represents hydrogen (including deuterium), an alkyl group having 1 to 6 carbon atoms, or a cycloalkyl group having 3 to 10 carbon atoms.

In the organic compound having any of the above structures, at least any one of R²⁵ to R²⁸ preferably represents an alkyl group having 1 to 6 carbon atoms or a cycloalkyl group having 3 to 10 carbon atoms.

In the organic compounds having any of the above structures, each of Q¹ to Q³ preferably represents N.

Another embodiment of the present invention is an organic compound represented by Structural Formula (100).

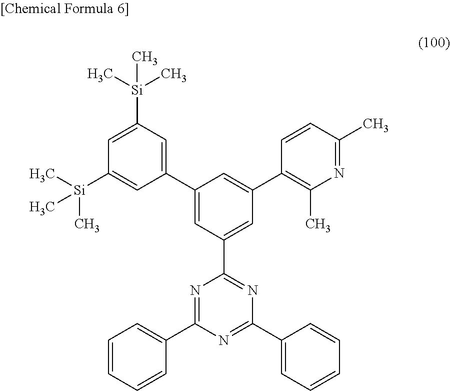

Another embodiment of the present invention is a light-emitting device including the organic compound having any of the above structures.

Another embodiment of the present invention is a light-emitting device including a first electrode, a second electrode, and an EL layer positioned between the first electrode and the second electrode, in which the EL layer includes a light-emitting layer and an electron-transport layer, the electron-transport layer is positioned between the light-emitting layer and the second electrode, the distance between the electron-transport layer and the second electrode is less than or equal to 5 nm, and the electron-transport layer includes the organic compound having any of the above structures.

Another embodiment of the present invention is a light-emitting device including a first electrode, a second electrode, and an EL layer positioned between the first electrode and the second electrode, in which the EL layer includes a light-emitting layer and an electron-transport layer, the electron-transport layer is positioned between the light-emitting layer and the second electrode, the electron-transport layer is a mixed layer including a first organic compound and a metal complex, and the first organic compound includes a π-electron deficient heteroaromatic ring and a trialkylsilyl group having 3 to 18 carbon atoms.

In the organic compounds having any of the above structures, the π-electron deficient heteroaromatic ring is preferably a pyrimidine ring or a triazine ring.

Another embodiment of the present invention is a light-emitting device including a first electrode, a second electrode, and an EL layer positioned between the first electrode and the second electrode, in which the EL layer includes a light-emitting layer and an electron-transport layer, the electron-transport layer is positioned between the light-emitting layer and the second electrode, the electron-transport layer is a mixed layer including a first organic compound and a metal complex, and the first organic compound is an organic compound represented by General Formula (G0).

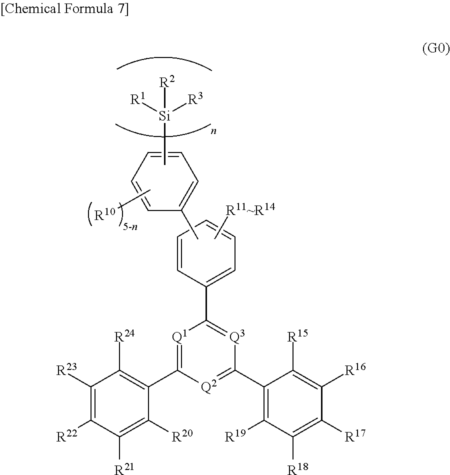

In General Formula (G0), each of Q¹ to Q³ independently represents N or CH; at least two of Q¹ to Q³ represent N; each of R¹ to R³ independently represents an alkyl group having 1 to 6 carbon atoms or a phenyl group; n is an integer greater than or equal to 1 and less than or equal to 5; each of R¹⁰ to R²⁴ independently represents hydrogen (including deuterium), an alkyl group having 1 to 6 carbon atoms, a cycloalkyl group having 3 to 10 carbon atoms, a trisubstituted silyl group having 3 to 18 carbon atoms, a substituted or unsubstituted aryl group having 6 to 30 carbon atoms, or a substituted or unsubstituted heteroaryl group having 3 to 30 carbon atoms; when n is greater than or equal to 2, a plurality of R¹'s may be the same or different from each other, a plurality of R²'s may be the same or different from each other, and a plurality of R³'s may be the same or different from each other; and when 5-n is greater than or equal to 2, a plurality of R¹⁰'s may be the same or different from each other.

In the organic compound having any of the above structures, the distance between the electron-transport layer and the second electrode is preferably less than or equal to 5 nm.

In the organic compound having any of the above structures, the metal complex preferably includes an alkali metal.

One embodiment of the present invention can provide an organic compound that has a low reflective index in the form of a film. Another embodiment can provide an organic compound that has a small GSP slope in the form of a film. Another embodiment can provide an organic compound that has a carrier-transport property. Another embodiment can provide an organic compound that has a high carrier-transport property, a low refractive index, and a small GSP slope in the form of a film. Another embodiment can provide a novel organic compound.

Another embodiment of the present invention can provide a light-emitting device having high emission efficiency. Another embodiment can provide a light-emitting device having a low driving voltage. Another embodiment can provide a light-emitting device, a light-emitting apparatus, an electronic appliance, a display device, and an electronic appliance each having low power consumption.

Note that the description of these effects does not preclude the existence of other effects. One embodiment of the present invention does not necessarily have all of these effects. Other effects can be derived from the description of the specification, the drawings, and the claims.

### BRIEF DESCRIPTION OF THE DRAWINGS

FIGS. 1A to 1F each illustrate a structure of a light-emitting device according to an embodiment.

FIGS. 2A and 2B are perspective views illustrating a structure example of a display module.

FIGS. 3A and 3B are cross-sectional views illustrating structure examples of a display device.

FIG. 4 is a perspective view illustrating a structure example of a display device.

FIG. 5 is a cross-sectional view illustrating a structure example of a display device.

FIG. 6 is a cross-sectional view illustrating a structure example of a display device.

FIGS. 7A and 7B illustrate examples of electronic appliances.

FIGS. 8A to 8F illustrate examples of electronic appliances.

FIGS. 9A to 9G illustrate examples of electronic appliances.

FIG. 10 illustrates a structure of a measurement device 1.

FIG. 11 illustrates a structure of a light-emitting device according to an example.

FIG. 12 illustrates a structure of a light-emitting device according to an example.

FIG. 13 is a ¹H-NMR chart of 2-[3-(2,6-dimethylpyridin-3-yl)-5-(4,6-diphenyl-1,3,5-triazine)]phenyl-4,4,5,5-tetramethyl-1,3,2-dioxaborolane.

FIG. 14 is an enlarged view of the ¹H-NMR chart of 2-[3-(2,6-dimethylpyridin-3-yl)-5-(4,6-diphenyl-1,3,5-triazine)]phenyl-4,4,5,5-tetramethyl-1,3,2-dioxaborolane.

FIG. 15 is a ¹H-NMR chart of mmTMSPh-mDMePyPTzn.

FIG. 16 is an enlarged view of a ¹H-NMR chart of mmTMSPh-mDMePyPTzn.

FIG. 17 shows an emission spectrum and an absorption spectrum of a dichloromethane solution of mmTMSPh-mDMePyPTzn.

FIG. 18 shows an emission spectrum and an absorption spectrum of a thin film of mmTMSPh-mDMePyPTzn.

FIG. 19 shows measurement data of refractive indices of mmTMSPh-mDMePyPTzn.

FIGS. 20A to 20C show a stable structure of mmTMSPh-mDMePyPTzn used for calculation.

FIGS. 21A to 21C show a stable structure of mmtBuPh-mDMePyPTzn used for calculation.

FIG. 22 shows capacity-voltage characteristics of a measurement device 1.

FIG. 23 shows current density-voltage characteristics of the measurement device 1.

FIG. 24 shows luminance-current density characteristics of a light-emitting device G-1, a comparative light-emitting device G-2, and a comparative light-emitting device G-3.

FIG. 25 shows luminance-voltage characteristics of the light-emitting device G-1 and the comparative light-emitting devices G-2 and G-3.

FIG. 26 shows current efficiency-luminance characteristics of the light-emitting device G-1 and the comparative light-emitting devices G-2 and G-3.

FIG. 27 shows current density-voltage characteristics of the light-emitting device G-1 and the comparative light-emitting device G-2.

FIG. 28 shows external quantum efficiency-luminance characteristics of the light-emitting device G-1 and the comparative light-emitting devices G-2 and G-3.

FIG. 29 shows electroluminescence spectra of the light-emitting device G-1 and the comparative light-emitting devices G-2 and G-3.

FIG. 30 shows capacity-voltage characteristics of the light-emitting device G-1 and the comparative light-emitting device G-2.

FIG. 31 shows luminance changes over driving time of the light-emitting device G-1 and the comparative light-emitting devices G-2 and G-3.

FIG. 32 shows luminance-current density characteristics of a light-emitting device G-4 and a comparative light-emitting device G-5.

FIG. 33 shows luminance-voltage characteristics of the light-emitting device G-4 and the comparative light-emitting device G-5.

FIG. 34 shows current efficiency-luminance characteristics of the light-emitting device G-4 and the comparative light-emitting device G-5.

FIG. 35 shows current density-voltage characteristics of the light-emitting device G-4 and the comparative light-emitting device G-5.

FIG. 36 shows external quantum efficiency-luminance characteristics of the light-emitting device G-4 and the comparative light-emitting device G-5.

FIG. 37 shows electroluminescence spectra of the light-emitting device G-4 and the comparative light-emitting device G-5.

FIG. 38 shows luminance-current density characteristics of a light-emitting device B-1 and a comparative light-emitting device B-2.

FIG. 39 shows luminance-voltage characteristics of the light-emitting device B-1 and the comparative light-emitting device B-2.

FIG. 40 shows current efficiency-luminance characteristics of the light-emitting device B-1 and the comparative light-emitting device B-2.

FIG. 41 shows current density-voltage characteristics of the light-emitting device B-1 and the comparative light-emitting device B-2.

FIG. 42 shows external quantum efficiency-luminance characteristics of the light-emitting device B-1 and the comparative light-emitting device B-2.

FIG. 43 shows blue index-luminance characteristics of the light-emitting device B-1 and the comparative light-emitting device B-2.

FIG. 44 shows electroluminescence spectra of the light-emitting device B-1 and the comparative light-emitting device B-2.

FIG. 45 shows luminance changes over driving time of the light-emitting device B-1 and the comparative light-emitting device B-2.

FIG. 46 shows luminance-current density characteristics of a light-emitting device B-3 and a comparative light-emitting device B-4 to a comparative light-emitting device B-6.

FIG. 47 shows luminance-voltage characteristics of the light-emitting device B-3 and the comparative light-emitting devices B-4 to B-6.

FIG. 48 shows current efficiency-luminance characteristics of the light-emitting device B-3 and the comparative light-emitting devices B-4 to B-6.

FIG. 49 shows current density-voltage characteristics of the light-emitting device B-3 and the comparative light-emitting devices B-4 to B-6.

FIG. 50 shows external quantum efficiency-luminance characteristics of the light-emitting device B-3 and the comparative light-emitting devices B-4 to B-6.

FIG. 51 shows blue index-luminance characteristics of the light-emitting device B-3 and the comparative light-emitting devices B-4 to B-6.

FIG. 52 shows electroluminescence spectra of the light-emitting device B-3 and the comparative light-emitting devices B-4 to B-6.

FIG. 53 is a ¹H-NMR chart of mmTMSPh-mPmPTzn.

FIG. 54 is an enlarged view of a ¹H-NMR chart of mmTMSPh-mPmPTzn.

FIG. 55 shows an emission spectrum and an absorption spectrum of mmTMSPh-mPmPTzn in a dichloromethane solution.

FIG. 56 shows an emission spectrum and an absorption spectrum of a thin film of mmTMSPh-mPmPTzn.

FIG. 57 shows measurement data of refractive indices of mmTMSPh-mPmPTzn.

FIG. 58 shows luminance-current density characteristics of a light-emitting device G-6, a comparative light-emitting device G-7, and a comparative light-emitting device G-8.

FIG. 59 shows luminance-voltage characteristics of the light-emitting device G-6 and the comparative light-emitting devices G-7 and G-8.

FIG. 60 shows current efficiency-luminance characteristics of the light-emitting device G-6 and the comparative light-emitting devices G-7 and G-8.

FIG. 61 shows current density-voltage characteristics of the light-emitting device G-6 and the comparative light-emitting devices G-7 and G-8.

FIG. 62 shows external quantum efficiency-luminance characteristics of the light-emitting device G-6 and the comparative light-emitting devices G-7 and G-8.

FIG. 63 shows electroluminescence spectra of the light-emitting device G-6 and the comparative light-emitting devices G-7 and G-8.

FIG. 64 shows luminance changes over driving time of the light-emitting device G-6 and the comparative light-emitting devices G-7 and G-8.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Thus, the present invention should not be construed as being limited to the description in the following embodiments.

Note that the position, size, range, or the like of each component illustrated in drawings and the like is not accurately represented in some cases for easy understanding. Thus, the disclosed invention is not necessarily limited to the position, size, range, or the like disclosed in the drawings and the like.

Ordinal numbers such as “first” and “second” in this specification and the like are used for convenience and do not denote the order of steps or the stacking order of layers in some cases. Thus, for example, description can be made even when “first” is replaced with “second” or “third”, as appropriate. In addition, the ordinal numbers in this specification and the like are not necessarily the same as those used to specify one embodiment of the present invention.

In the description of structures of the present invention in this specification and the like with reference to the drawings, the same components in different drawings are denoted by the same reference numeral in some cases.

In this specification and the like, the terms “film” and “layer” can be interchanged with each other. For example, the term “conductive layer” can be changed to the term “conductive film” in some cases. For another example, the term “insulating film” can be changed into the term “insulating layer” in some cases.

Note that in this specification and the like, hydrogen (H) includes protium (¹H) and deuterium (²H or D). Protium is a stable hydrogen isotope having a mass number of 1. Deuterium is a stable hydrogen isotope having a mass number of 2.

### Embodiment 1

In this embodiment, an organic compound of one embodiment of the present invention will be described.

One embodiment of the present invention is an organic compound including a trisubstituted silyl group. In this specification and the like, a trisubstituted silyl group refers to a monovalent group having a structure in which three substituents are bonded to a silicon atom (Si). Specific examples of the three substituents include an alkyl group and an aryl group. Note that an alkyl group is preferable to an aryl group because the refractive index of the organic compound with an alkyl group can be made lower than that of the organic compound with an aryl group.

In order to examine the effects of introduction of a trisubstituted silyl group into an organic compound, the present inventors analyzed the permanent electric dipole moments of the stable structures of trimethylsilylbenzene in the singlet ground state and tert-butylbenzene in the singlet ground state. Note that trimethylsilylbenzene was analyzed as the organic compound including a trisubstituted silyl group and tert-butylbenzene was analyzed as a comparative example. Since a trimethylsilyl group and a tert-butyl group include saturated hydrocarbon groups, introduction of a trimethylsilyl group or a tert-butyl group into an aromatic ring of an organic compound is expected to reduce the refractive index of the organic compound.

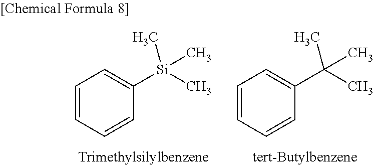

A density functional theory (DFT) method was used as the calculation method. As a functional, B3LYP was used, and as a basis function, 6-311G(d,p) was used. As a computational program, Gaussian 16 was used.

As a result of the calculation, the magnitude of the permanent electric dipole moment of tert-butylbenzene is 0.3110 Debye. This permanent electric dipole moment results from donation of an electron to the benzene ring from a tert-butyl group, which is an electron-donating group. By contrast, the magnitude of the permanent electric dipole moment of trimethylsilylbenzene is 0.0345 Debye, which is less than that of the tert-butylbenzene and close to zero. This indicates that the trimethylsilyl group hardly exhibits an electron-donating property with respect to the benzene ring. Therefore, it can be said that a trisubstituted silyl group has a pooler electron-donating property and is accordingly more effective in reducing the permanent electric dipole moment of a molecule than a group having a structure in which the same three substituents as those in the trisubstituted silyl group are bonded to a carbon atom.

By this effect, a molecule for an organic semiconductor that includes a trisubstituted silyl group is capable of maintaining a small permanent electric dipole moment. Accordingly, such a molecule can be formed into a film with a small SOP by a vacuum evaporation method.

In view of the above, as one embodiment of the present invention, the present inventors have developed an organic compound where a trisubstituted silyl group is introduced into an electron-transport skeleton. The electron-transport skeleton includes a pyrimidine ring or a triazine ring that is a π-electron deficient heteroaromatic ring and three phenyl groups bonded to the ring. Any of the three phenyl groups includes a substituent including a trisubstituted silyl group. The organic compound has an electron-transport property and an evaporated film of the organic compound has a low refractive index and a small GSP slope. When the organic compound is used for an electron-transport layer of a light-emitting device, the emission efficiency of the light-emitting device can be improved.

Next, the organic compound of one embodiment of the present invention is more specifically described using general formulae. One embodiment of the present invention is an organic compound represented by General Formula (G1).

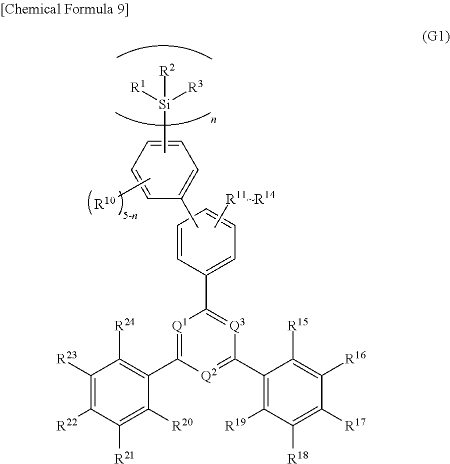

In General Formula (G1), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹¹ to R²⁴ independently represents hydrogen (including deuterium), an alkyl group having 1 to 6 carbon atoms, a cycloalkyl group having 3 to 10 carbon atoms, a substituted or unsubstituted aryl group having 6 to 30 carbon atoms, or a substituted or unsubstituted heteroaryl group having 3 to 30 carbon atoms; at least any one of R¹¹ to R²⁴ represents a substituted or unsubstituted heteroaryl group having 3 to 30 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

Note that CH represents carbon (C) to which hydrogen (H) is bonded, and CD represents carbon (C) to which deuterium (D) is bonded.

In the case of a structure where a substituent including a trisubstituted silyl group is bonded to only any one of the three phenyl groups bonded to the pyrimidine ring or the triazine ring as in the organic compound represented by General Formula (G1), an increase in steric hindrance around the pyrimidine ring or the triazine ring can be inhibited so that the electron-transport property of the organic compound can be improved, as compared with the case where the substituent is bonded to two or more of the three phenyl groups bonded to the pyrimidine ring or the triazine ring.

In the case of a structure where a substituted or unsubstituted heteroaryl group having 3 to 30 carbon atoms is bonded to at least any one of the three phenyl groups bonded to the pyrimidine ring or the triazine ring as in the organic compound represented by General Formula (G1), the electron-transport property of the organic compound can be further improved.

The molecular weight of the organic compound represented by General Formula (G1) is preferably lower than or equal to 1500, further preferably lower than or equal to 1000, in which case it is expected that deposition by vacuum evaporation can be performed at a temperature lower than the thermal decomposition temperature of the organic compound.

Another embodiment of the present invention is an organic compound represented by General Formula (G2).

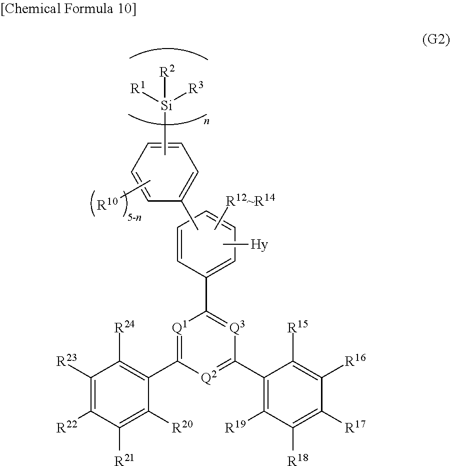

In General Formula (G2), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; Hy represents a substituted or unsubstituted heteroaryl group having 3 to 30 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

General Formula (G2) is different from General Formula (G1) in that both the substituent including a trisubstituted silyl group and the substituted or unsubstituted heteroaryl group having 3 to 30 carbon atoms are bonded to one of the three phenyl groups bonded to the pyrimidine ring or the triazine ring. With such a structure, a film of the organic compound can have an improved electron-transport property and a reduced GSP slope as compared with the case where the substituent and the heteroaryl group are bonded to respective phenyl groups.

In each of the organic compounds represented by the above general formulae, the substituted or unsubstituted heteroaryl group having 3 to 30 carbon atoms is preferably a group including nitrogen, further preferably a group in which one or more of the atoms included in an aromatic ring are further preferably nitrogen atoms, still further preferably a substituted or unsubstituted pyridinyl group, a substituted or unsubstituted pyrimidinyl group, or a substituted or unsubstituted pyrazinyl group. These groups have a high electron-transport property, which further improves the electron-transport property of the organic compound. These groups are preferable also because they exhibit no absorption in the visible range and can improve the efficiency of a light-emitting device.

In each of the organic compounds represented by the above general formulae, the substituted or unsubstituted heteroaryl group having 3 to 30 carbon atoms preferably includes at least any one of an alkyl group having 1 to 6 carbon atoms and a cycloalkyl group having 3 to 10 carbon atoms. In this case, the refractive index of a film of the organic compound can be further lowered and the GSP slope of the film can be further reduced.

Another embodiment of the present invention is an organic compound represented by General Formula (G3).

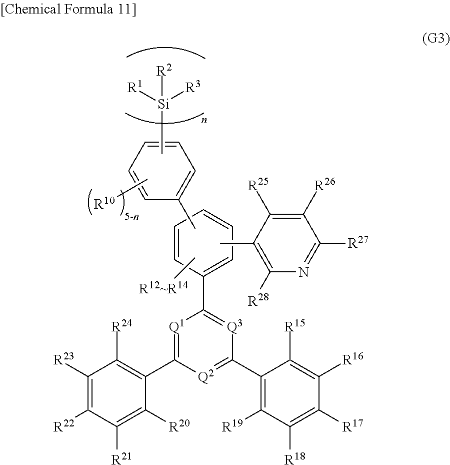

In General Formula (G3), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; each of R²⁵ to R²⁸ independently represents hydrogen (including deuterium), an alkyl group having 1 to 6 carbon atoms, or a cycloalkyl group having 3 to 10 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

General Formula (G3) is different from General Formula (G2) in that Hy in General Formula (G2) is limited to a substituted or unsubstituted pyridinyl group. This can further improve the electron-transport property of the organic compound while maintaining the low refractive index.

Another embodiment of the present invention is an organic compound represented by General Formula (G4).

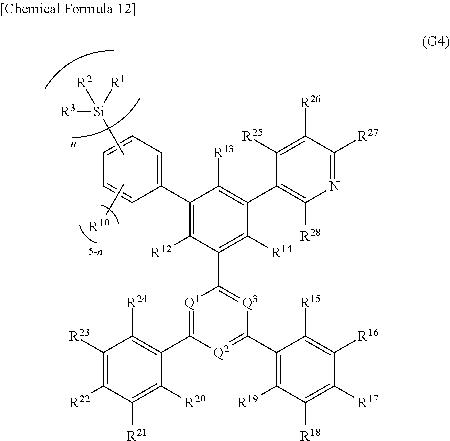

In General Formula (G4), each of Q¹ to Q³ independently represents N or CH (including CD); at least two of Q¹ to Q³ represent N; each of R¹ to R³ independently represents an alkyl group having 1 to 6 carbon atoms or a phenyl group; n is an integer greater than or equal to 2 and less than or equal to 5; R¹⁰ represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; each of R²⁵ to R²⁸ independently represents hydrogen (including deuterium), an alkyl group having 1 to 6 carbon atoms, or a cycloalkyl group having 3 to 10 carbon atoms; a plurality of R¹'s may be the same or different from each other; a plurality of R²'s may be the same or different from each other; a plurality of R³'s may be the same or different from each other; and when 5-n is greater than or equal to 2, a plurality of R¹⁰'s may be the same or different from each other.

General Formula (G4) is different from General Formula (G3) in that the substitution sites of the phenyl group including a trisubstituted silyl group and the substituted or unsubstituted pyridinyl group, are limited to be meta positions with respect to the triazine or pyrimidine. The structure where the phenyl group including a trisubstituted silyl group and the substituted or unsubstituted pyridinyl group are at the substitution sites shown in General Formula (G4) can inhibit steric hindrance due to the proximity of the substituents, improving the stability of the molecule. In addition, a reduction in LUMO level can be avoided unlike in the case where the groups are substituted at the para-positions. Such a structure where the substitution sites are limited to the meta-positions is preferably employed also because it can inhibit extension of the conjugation of the unsaturated bond to further reduce the refractive index. In this case, R¹² to R¹⁴ are preferably hydrogen (including deuterium) to enable higher stability.

In each of the organic compounds represented by the above general formulae, n is preferably 2 or 3, further preferably 2. In this case, it is possible to prevent steric hindrance due to the proximity of the trisubstituted silyl groups and resultant instability of the molecule. In this case, R¹ to R¹⁰ are preferably hydrogen (including deuterium) to enable higher stability.

Another embodiment of the present invention is an organic compound represented by General Formula (G5).

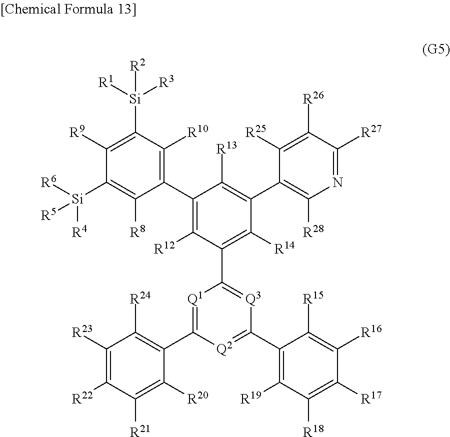

In General Formula (G5), each of Q¹ to Q³ independently represents N or CH (including CD); at least two of Q¹ to Q³ represent N; each of R¹ to R⁶ independently represents an alkyl group having 1 to 6 carbon atoms or a phenyl group; each of R⁸ to R¹⁰ independently represents hydrogen (including deuterium), an alkyl group having 1 to 6 carbon atoms, or a cycloalkyl group having 3 to 10 carbon atoms; each of R¹² to R²⁴ independently represents hydrogen (including deuterium), an alkyl group having 1 to 6 carbon atoms, a cycloalkyl group having 3 to 10 carbon atoms, or a substituted or unsubstituted aryl group having 6 to 30 carbon atoms; and each of R²⁵ to R²⁸ independently represents hydrogen (including deuterium), an alkyl group having 1 to 6 carbon atoms, or a cycloalkyl group having 3 to 10 carbon atoms.

General Formula (G5) is different from General Formula (G4) in that the number (n) and the substitution sites of the trisubstituted silyl groups in General Formula (G4) are limited. The structure including two trisubstituted silyl groups in the substitution sites shown in General Formula (G5) can prevent steric hindrance due to the proximity of trisubstituted silyl groups and resultant instability of the molecule; thus, the organic compound can be stable.

In the organic compound represented by any of the above general formulae, at least any one of R²⁵ to R²⁸ preferably represents an alkyl group having 1 to 6 carbon atoms or a cycloalkyl group having 3 to 10 carbon atoms. This can reduce the refractive index of the organic compound.

In the organic compound represented by any of the above general formulae, each of Q¹ to Q³ preferably represents N. This can increase the electron-transport property of the organic compound.

Next, specific examples of substituents that can be used for the organic compounds represented by the above general formulae will be described. Note that groups that can be used in the above general formulae are not limited to the following specific examples. In addition, in the specific examples described below, some or all of hydrogen atoms may be deuterium.

### <<Alkyl Group Having 1 to 6 Carbon Atoms>>

An alkyl group having 1 to 6 carbon atoms is a monovalent group obtained by removing one hydrogen (H) from an alkane having 1 to 6 carbon atoms. Specific examples include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, a 2,3-dimethylbutyl group, and the like. The alkyl group is preferably a methyl group, in which case there is less steric hindrance due to the proximity of the substituents (alkyl groups), contributing to the higher stability of the molecule. A methyl group is preferably used also because it is sterically small among alkyl groups and therefore less likely to impede carrier transport. In the case where the alkyl group has two or more carbon atoms, the refractive index of the organic compound can be further reduced.

### <<Cycloalkyl Group Having 3 to 10 Carbon Atoms>>

A cycloalkyl group having 3 to 10 carbon atoms is a monovalent group obtained by removing one hydrogen from a monocyclic or polycyclic cycloalkane having 3 to 10 carbon atoms. Specific examples include a cyclopropyl group, a cyclobutyl group, a cyclopentyl group, a cyclohexyl group, a 4-methylcyclohexyl group, a cycloheptyl group, a cyclooctyl group, a cyclononyl group, a cyclodecyl group, a norbornyl group, a bicyclo[2,2,2]octyl group, a decahydronaphthyl group, an adamantyl group, and the like. A cycloalkyl group having 6 or more carbon atoms is preferably used, in which case the refractive index of the organic compound can be lower and the glass transition temperature (Tg) of the organic compound can be higher than in the case where a cycloalkyl group having 5 or less carbon atoms is used. In particular, a cyclohexyl group is preferable because it is inexpensive.

### <<Aryl Group Having 6 to 30 Carbon Atoms>>

An aryl group having 6 to 30 carbon atoms is a monovalent group obtained by removing one hydrogen from one of carbon atoms forming a ring of a monocyclic or polycyclic aromatic compound having 6 to 30 carbon atoms. Specific examples include a phenyl group, an o-tolyl group, a m-tolyl group, ap-tolyl group, a mesityl group, a biphenyl-2-yl group (o-biphenyl group), a biphenyl-3-yl group (m-biphenyl group), a biphenyl-4-yl group (p-biphenyl group), a 1-naphthyl group, a 2-naphthyl group, a phenylnaphthyl group, a naphthylphenyl group, a terphenyl group, a fluorenyl group, a 9,9-dimethylfluorenyl group, a quaterphenyl group, a spirobifluorenyl group, a phenanthryl group, an anthryl group, a binaphthylphenyl group, a fluoranthenyl group, and the like. In the case where the aryl group having 6 to 30 carbon atoms includes a substituent, specific examples of the substituent include an alkyl group having 1 to 6 carbon atoms, a cycloalkyl group having 3 to 10 carbon atoms, and a phenyl group. Among aryl groups, an aryl group having a six-membered ring is preferable because of its high stability and high reliability.

### <<Heteroaryl Group Having 2 to 30 Carbon Atoms>>

A heteroaryl group having 2 to 30 carbon atoms is a monovalent group obtained by removing one hydrogen from one of carbon atoms forming a ring of a monocyclic or polycyclic heterocyclic aromatic compound having 2 to 30 carbon atoms. Specific examples include a carbazolyl group, a dibenzothiophenyl group, a dibenzofuranyl group, a benzocarbazolyl group, a naphthobenzothiophenyl group, a naphthobenzofuranyl group, a dibenzocarbazolyl group, a dinaphthothiophenyl group, a dinaphthofuranyl group, a triazinyl group, a pyrimidinyl group, a pyrazinyl group, a triazolyl group, a pyridinyl group, a benzofuropyrimidinyl group, a benzothiopyrimidinyl group, a benzofuropyrazinyl group, a benzothiopyrazinyl group, a benzofuropyridinyl group, a benzothiopyridinyl group, a bicarbazolyl group, and the like. In the case where the heteroaryl group having 2 to 30 carbon atoms includes a substituent, examples of the substituent include an alkyl group having 1 to 6 carbon atoms, a cycloalkyl group having 3 to 10 carbon atoms, and a phenyl group. Among heteroaryl groups, a heteroaryl group having a six-membered ring is preferable because of its high stability and high reliability. A heteroaryl group having two or more nitrogens is preferable because it improves the electron-transport property. A heteroaryl group having one nitrogen is preferable because it reduces the refractive index.

The above substituents are specific examples of the substituent that can be used for the organic compounds represented by the general formulae.

Specific examples of the organic compounds of embodiments of the present invention represented by the above general formulae include organic compounds represented by Structural Formulae (100) to (122) below. Note that the organic compound of one embodiment of the present invention is not limited to the organic compounds represented by the following structural formulae.

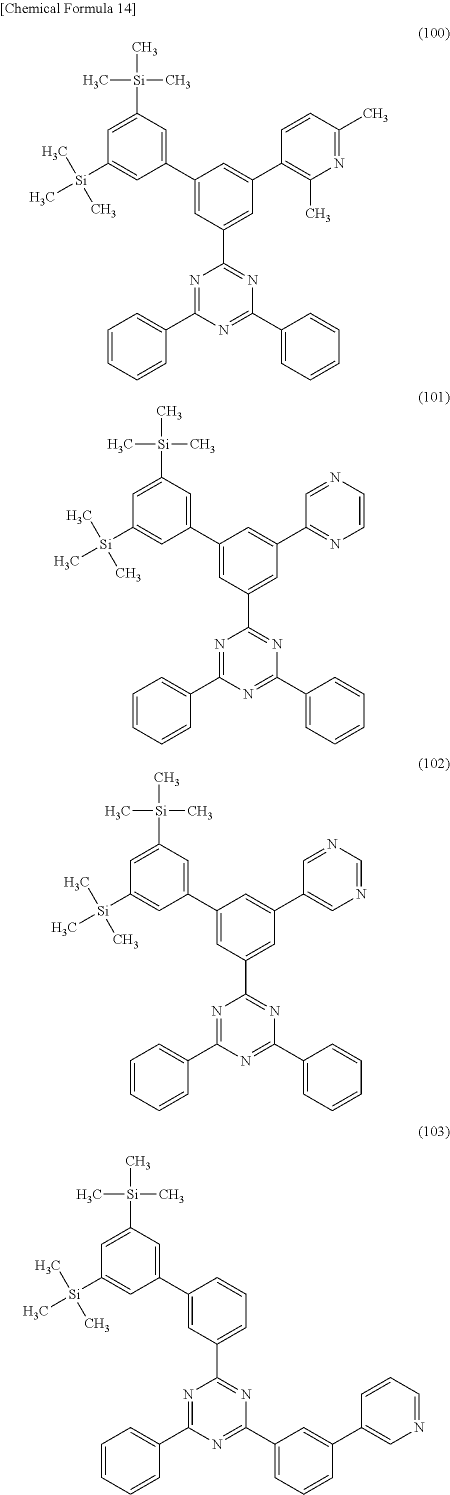

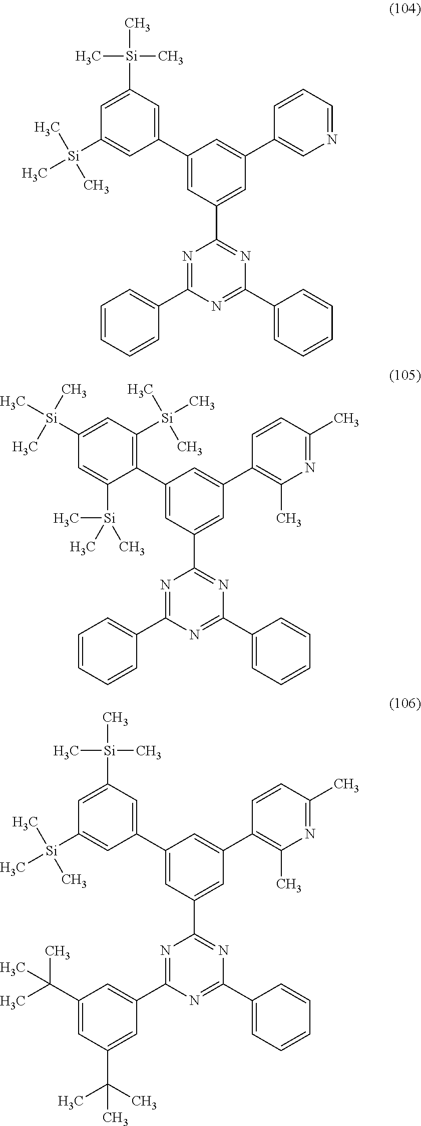

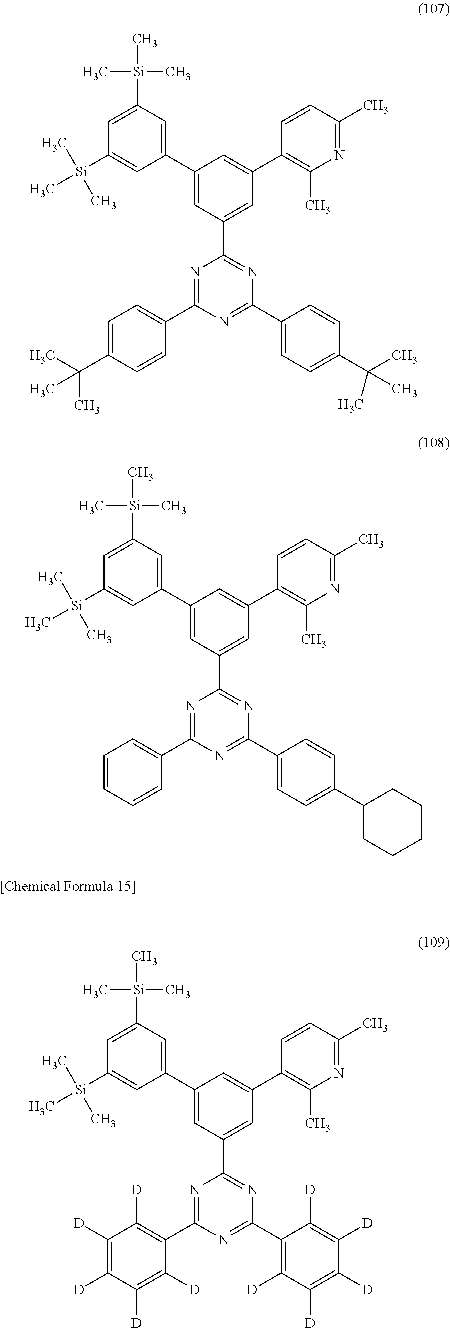

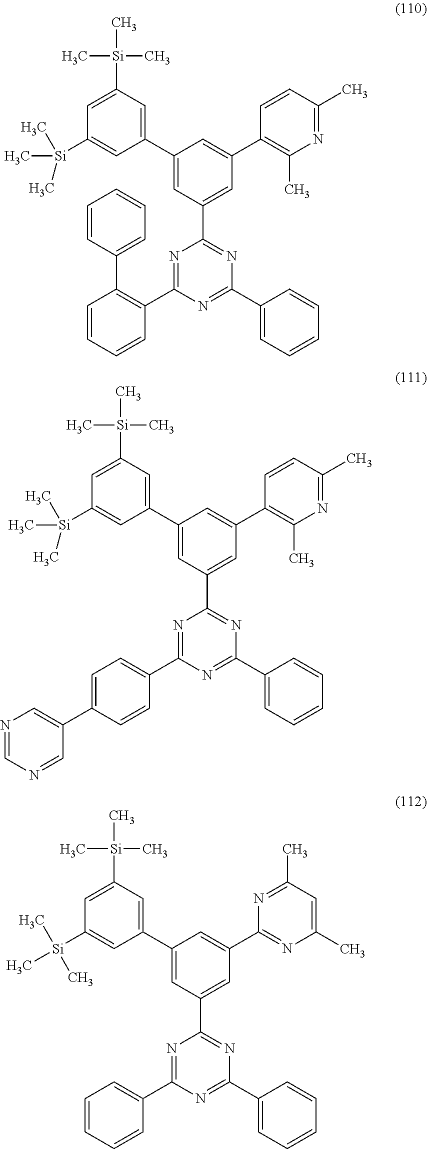

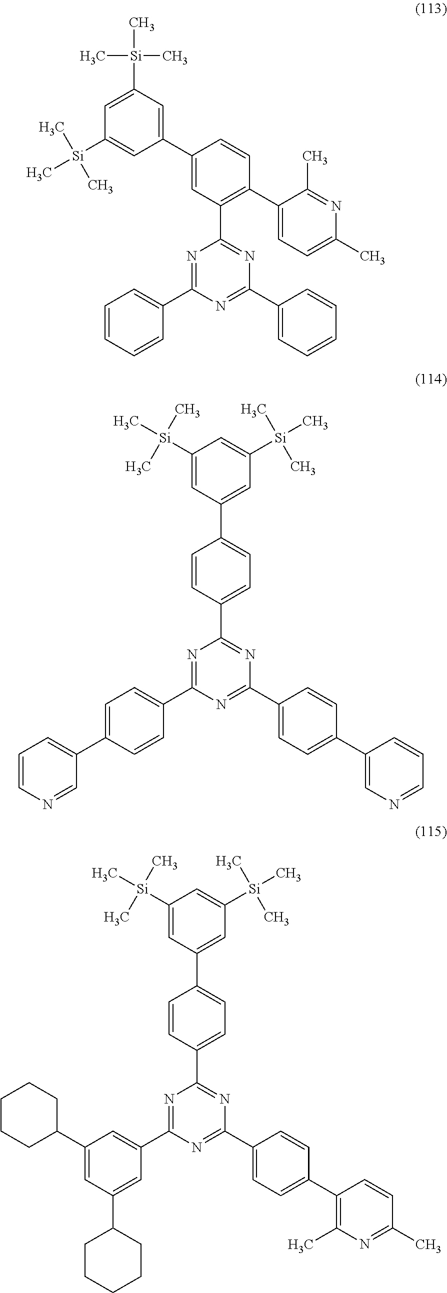

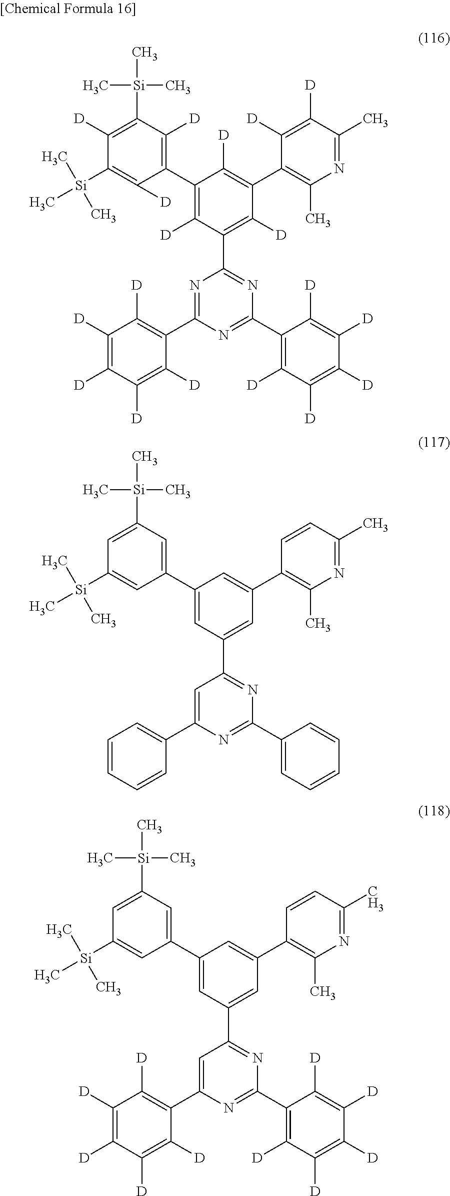

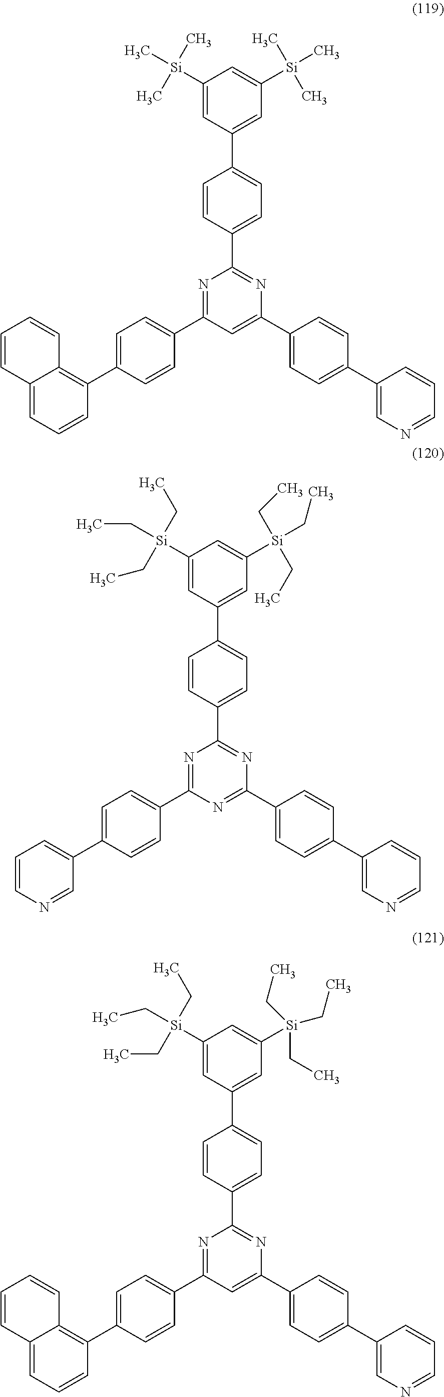

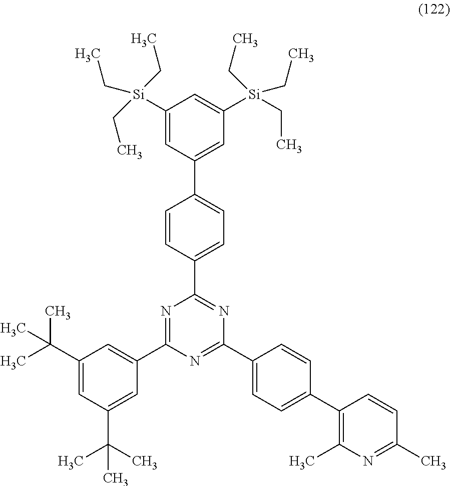

Next, as an example of a method of synthesizing the organic compound of one embodiment of the present invention, a method of synthesizing the organic compounds represented by General Formula (G1) is described.

The organic compound represented by General Formula (G1) can be obtained in the following manner: a halogen compound (B1) is used to synthesize a boron compound (B2) as shown in Synthesis Scheme (A-1); then, the boron compound (B2) and a halogen compound including Si (B3) are reacted as shown in Synthesis Scheme (A-2).

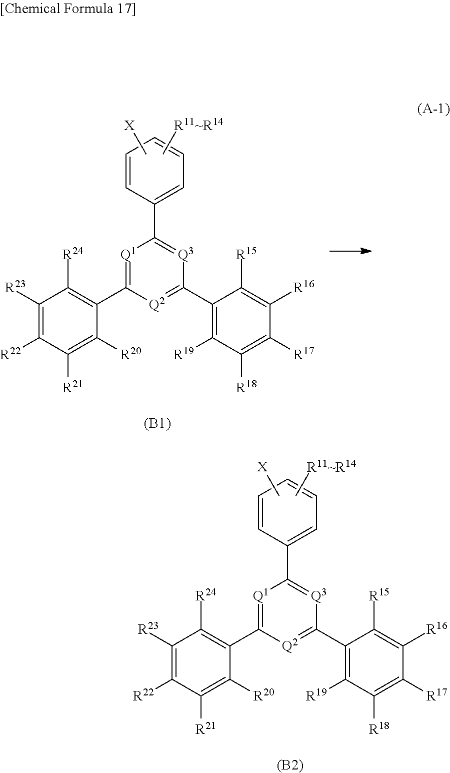

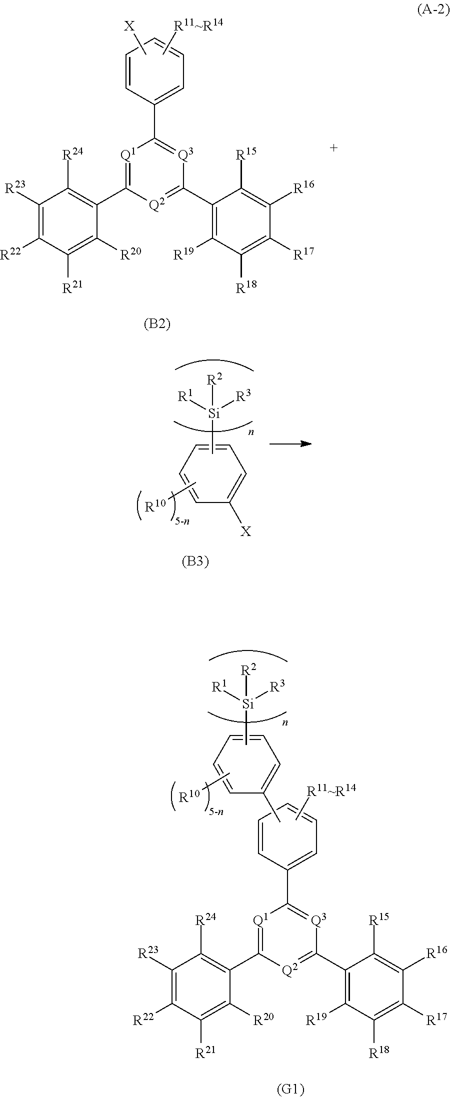

Note that Q¹ to Q³, R¹ to R³, n, R¹⁰, and R¹¹ to R²⁴ in Synthesis Schemes (A-1) and (A-2) are the same as those in the above description of General Formula (G1) and are not described here. X represents chlorine, bromine, iodine, or a sulfonyloxy group. When X is a halogen, it preferably has a large atomic number to increase the reactivity of the halogen compound (B1). Y represents a boronyl group. Note that as the boron compound (B2), a boronic ester such as pinacol boronic ester may be used.

In Synthesis Scheme (A-1) above, the halogen compound (B1) and a boron source are coupled in the presence of a palladium catalyst, whereby the boron compound (B2) can be synthesized. Examples of the boron source include bis(pinacolato)diboron and pinacol borane. Examples of the palladium catalyst include [1,1′-bis(diphenylphosphino)ferrocene]palladium(II) dichloride dichloromethane adduct, palladium(II) acetate, and tris(dibenzylideneacetone)dipalladium(0). Examples of a ligand of the palladium catalyst include 2-dicyclohexylphosphino-2′,4′,6′-triisopropylbiphenyl. Examples of a base include inorganic bases such as potassium acetate, potassium carbonate, and tripotassium phosphate. Examples of a solvent include dimethyl sulfoxide, N,N-dimethylformamide, and 1,4-dioxane. Reagents that can be used are not limited to these.

Note that in Synthesis Scheme (A-1) above, the boron compound (B2) can also be obtained by reacting the halogen compound (B1), a lithium reagent, and borate ester.

In Synthesis Scheme (A-2) above, the boron compound (B2) and the halogen compound including Si (B3) are coupled in the presence of a palladium catalyst, whereby the organic compound represented by General Formula (G1) can be synthesized. Examples of the palladium catalyst include tetrakis(triphenylphosphine)palladium(0), palladium(II) acetate, and tris(dibenzylideneacetone)dipalladium(0). Examples of a ligand of the palladium catalyst include 2-dicyclohexylphosphino-2′,4′,6′-triisopropylbiphenyl, 2-dicyclohexylphosphino-2′,6′-dimethoxybiphenyl, tri(ortho-tolyl)phosphine, and triphenylphosphine. Examples of a base include inorganic bases such as potassium acetate, potassium carbonate, sodium carbonate, and tripotassium phosphate. Examples of a solvent include toluene, xylene, tetrahydrofuran, dioxane, ethylene glycol dimethyl ether, ethanol, and water. Reagents that can be used are not limited to these.

The organic compound of one embodiment of the present invention can be synthesized in the above manner, but the present invention is not limited thereto, and any other synthesis method may be employed.

### <Method for Obtaining GSP Slope>

Here, a method for obtaining the GSP slope of an organic compound film formed by a vacuum evaporation method is described.

A phenomenon in which the surface potential of an evaporated film increases in proportion to the thickness of the film is called the giant surface potential as described above. In general, a slope of a plot of the surface potential of an evaporated film in the thickness direction by Kelvin probe measurement is assumed as the level of the giant surface potential, that is, GSP slope (mV/nm); in the case where two different layers are stacked, a change in the density of charges (mC/m²) accumulated at the interface, which is in association with GSP, can be utilized to estimate the GSP slope. The density of the charges accumulated at the interface is obtained by C-V (capacity-voltage) measurement on an element structure in which charges are accumulated on one of the layers.

When a voltage is applied to a stack of organic thin films with different SOPs (a thin film 1 on the anode side and a thin film 2 on the cathode side; the anode is closer to the substrate than the cathode) and carriers accumulated at the interface (accumulated charges) are electrons, the following equations hold.

$$\begin{matrix}[ Equation 1 ] & \\ {σ}_{acc} = ( {V}_{th} - {V}_{inj} ) \frac{{ε}_{1}}{{d}_{1}} = {σ}_{int} & ( 1 )\end{matrix}$$

$$\begin{matrix}[ Equation 2 ] & \\ {σ}_{int} = {P}_{1} - {P}_{2} = \frac{{ε}_{1} {V}_{1}}{{d}_{1}} - \frac{{ε}_{2} {V}_{2}}{{d}_{2}} & ( 2 )\end{matrix}$$

In Equation (1), σ_{acc} is an accumulated charge density, σᵢₙₜ is an interface charge density, Vᵢₙⱼ is an electron-injection voltage, Vₜₕ is a threshold voltage, d₁ is the thickness of the thin film 1, and ε₁ is the dielectric constant of the thin film 1. Note that Vᵢₙⱼ and Vₜₕ can be estimated from the capacity-voltage characteristics of a device. As the dielectric constant, it is possible to use a value obtained by multiplying the vacuum permittivity and the relative permittivity, which is assumed to be the square of the ordinary refractive index nₒ (the value at a wavelength of 633 nm). As described above, according to Equation (1), the interface charge density σᵢₙₜ can be calculated using Vᵢₙⱼ and Vₜₕ estimated from the capacity-voltage characteristics, the dielectric constant ε₁ of the thin film 1 calculated from the refractive index, and the thickness d₁ of the thin film 1.

Next, in Equation (2), P₁ and P₂ are the degree of SOP of the thin film 1 and the degree of SOP of the thin film 2, respectively, in the direction perpendicular to the substrate surface, ε₂ is the dielectric constant of the thin film 2, and d₂ is the thickness of the thin film 2. Since the interface charge density σᵢₙₜ can be obtained from Equation (1) above, the use of a substance with a known GSP slope for the thin film 1 enables the GSP slope of the thin film 2 to be estimated.

The following is an example of fabricating a measurement device 1 using tris(8-quinolinolato)aluminum(III) (abbreviation: Alq₃) whose GSP slope is known, for the thin film 1 and mmTMSPh-mDMePyPTzn, which is an organic compound of one embodiment of the present invention, for the thin film 2. Note that the GSP slope of Alq₃ measured using a Kelvin probe is 48 mV/nm according to Non-Patent Document 3. Chemical formulae of mmTMSPh-mDMePyPTzn and Alq₃ are shown below.

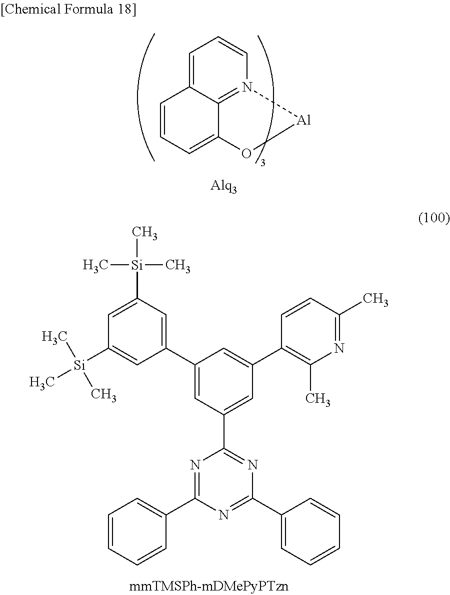

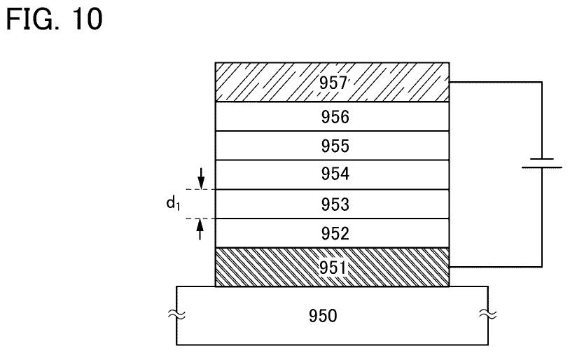

As illustrated in FIG. 10, the measurement device 1 has a structure in which a layer 952, a layer 953, a layer 954, a layer 955, and a layer 956 are stacked in this order over an anode 951 formed over a glass substrate 950, and a cathode 957 is stacked over the layer 956. Table 1 shows the device structure of the measurement device 1.

The layer 953 and the layer 954 in the measurement device 1 correspond to the thin film 1 and the thin film 2, respectively. The layer 952 was formed by co-evaporation of N-(biphenyl-4-yl)-N-[4-(9-phenyl-9H-carbazol-3-yl)phenyl]-9,9-dimethyl-9H-fluoren-2-amine (abbreviation: PCBBiF) and OCHD-003, which is an organic compound having an electron-acceptor property, to a thickness of 10 nm at the weight ratio of 1:0.1 (PCBBiF: OCHD-003). The layer 953 was formed by evaporation of Alq₃ to a thickness of 100 nm. The layer 954 was formed by evaporation of mmTMSPh-mDMePyPTzn to a thickness of 100 nm. The layer 955 was formed by evaporation of 4,7-di-1-pyrrolidinyl-1,10-phenanthroline (abbreviation: Pyrrd-Phen) to a thickness of 1 nm.

TABLE 1

| | Thickness | | Measurement device 1 |
|---|---|---|---|
| Cathode 957 | 200 | nm | Al |
| Layer 956 | 1 | nm | LiF |
| Layer 955 | 1 | nm | Pyrrd-Phen |
| Layer 954 | 100 | nm | mmTMSPh-mDMePyPTzn |
| Layer 953 | 100 | nm | Alq₃ |
| Layer 952 | 10 | nm | PCBBiF:OCHD-003 (1:0.1) |
| Anode 951 | 110 | nm | ITSO |

Note that layers 952 to 956 and the cathode in each measurement device were formed from the anode side by a vacuum evaporation method under the conditions where the substrate temperature was set to room temperature and the deposition rate ranged from 0.2 nm/s to 0.6 nm/s. Each layer was formed without interruption of evaporation. In fabrication of the measurement device, the deposition rate of each layer is preferably within the range of 3 nm/min to 600 nm/min. The thickness of each layer in the measurement device is preferably greater than or equal to 1 nm and less than or equal to 500 nm, further preferably greater than or equal to 50 nm and less than or equal to 300 nm.

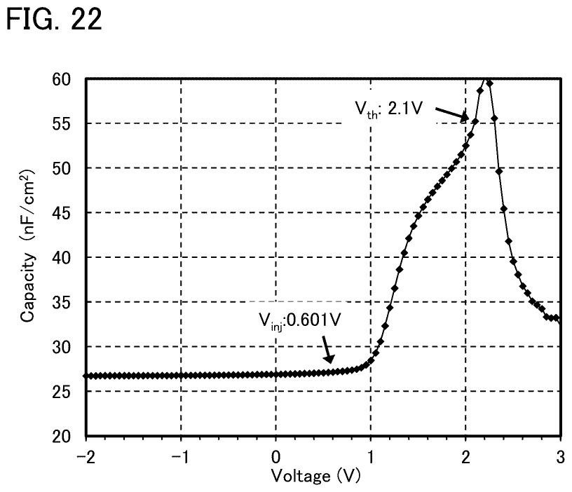

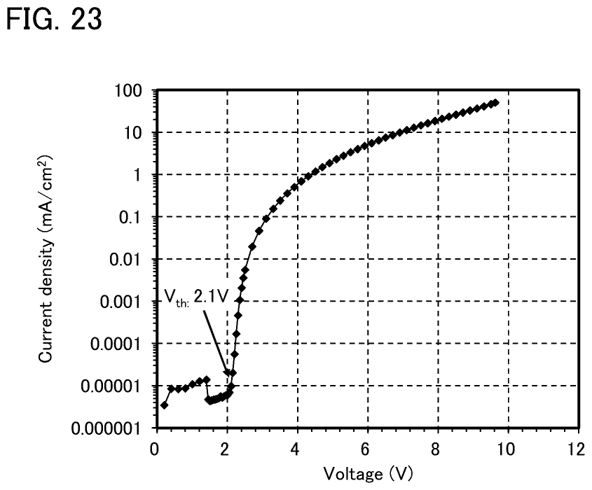

FIG. 22 shows capacity-voltage characteristics of the measurement device 1 and FIG. 23 shows current density-voltage characteristics thereof.

Table 2 shows the electron-injection voltage Vᵢₙⱼ, the interface charge density σᵢₙₜ, and the GSP slope of the measurement device 1, which were obtained from FIG. 22 and Equations (1) and (2), and the ordinary refractive indices nₒ of Alq₃ and mmTMSPh-mDMePyPTzn, the actually measured thickness d₁ of the layer 953, and the threshold voltage Vₜₕ obtained from FIG. 23, which were used in the calculation. Note that the threshold voltage Vₜₕ can also be determined from the capacity-voltage characteristics. As the actually measured thickness d₁ of the layer 953, a highly accurate value of an actually formed film calculated by a spectroscopic ellipsometry method was used.

TABLE 2

| | Measurement |
|---|---|
| | device 1 |
| Electron-injection voltage Vᵢₙⱼ (V) | 0.601 |
| Threshold voltage Vₜₕ (V) | 2.1 |
| Interface charge density σᵢₙₜ (mC/m²) | 0.45 |
| Ordinary refractive index nₒ of Alq₃ (@633 nm) | 1.71 |
| Ordinary refractive index nₒ of | 1.60 |
| mmTMSPh-mDMePyPTzn (@633 nm) | |
| d₁ (nm) | 85.95 |
| GSP slope (mV/nm) | 35.0 |

The GSP slope was estimated to be 35.0 mV/nm, as shown in Table 2, from the measurement results of the measurement device 1. That is, the GSP slope had a preferable value, which is less than or equal to 40.0 mV/nm.

In this manner, a device in which Alq₃ with a known GSP slope and an organic compound film whose GSP slope is to be obtained are stacked is fabricated and the capacity-voltage characteristics are measured, so that the GSP slope of the organic compound can be estimated.

In the above, the method for calculating the GSP slope of the organic compound used for the electron-transport layer in which electrons serve as carriers is described. In the case of using the GSP slope of an organic compound used for a hole-transport layer in which holes serve as carriers, the GSP slope can be calculated in a similar manner using Equation (3) shown below, as described in Non-Patent Document 4.

$$\begin{matrix}[ Equation 3 ] & \\ {σ}_{acc} = ( {V}_{th} - {V}_{inj} ) \frac{{ε}_{2}}{{d}_{2}} = - {σ}_{int} & ( 3 )\end{matrix}$$

Organic compounds used for layers of a light-emitting device are preferably selected in consideration of the GSP slopes of evaporated films of the organic compounds, which are measured in advance by the above measurement method.

The structures described in this embodiment can be used in appropriate combination with any of the structures described in the other embodiments.

### Embodiment 2

In this embodiment, a light-emitting device of one embodiment of the present invention is described.

In the light-emitting device of one embodiment of the present invention, an organic compound where a trisubstituted silyl group is introduced into an electron-transport skeleton can be used. Specifically, an organic compound including the π-electron deficient heteroaromatic ring and a trisubstituted silyl group having 3 to 18 carbon atoms can be used in the light-emitting device of one embodiment of the present invention. As described in Embodiment 1, since such an organic compound has an electron-transport property and its evaporated film has a low refractive index and a small GSP slope, the light-emitting device using the organic compound for an electron-transport layer can have increased emission efficiency and reduced driving voltage.

In the case where the organic compound is used for the electron-transport layer of a light-emitting device, the electron-transport layer is preferably a mixed layer including the organic compound and a metal complex, in which case the property of electron injection into the electron-transport layer is further improved.

The trisubstituted silyl group having 3 to 18 carbon atoms in the organic compound is a group having a structure in which three alkyl groups having 3 to 18 carbon atoms in total or three aryl groups having 3 to 18 carbon atoms in total are bonded to silicon (Si). Specific examples of a silyl group having 3 to 18 carbon atoms include a trimethylsilyl group, a triethylsilyl group, a tert-butyldimethylsilyl group, a triphenylsilyl group, and the like. In particular, a trialkylsilyl group having 3 to 18 carbon atoms, such as a trimethylsilyl group, a triethylsilyl group, or a tert-butyl dimethylsilyl group, is preferable because it can further reduce the refractive index of the organic compound. The organic compound including a trimethylsilyl group is preferable particularly because it can be inexpensively synthesized.

Examples of the π-electron deficient heteroaromatic ring in the organic compound include an oxadiazole ring, a triazole ring, a benzimidazole ring, a quinoxaline ring, a dibenzoquinoxaline ring, a quinazoline ring, a phenanthroline ring, a pyridine ring, a diazine ring (a pyrimidine ring, a pyrazine ring, and a pyridazine ring), a triazine ring, and a furodiazine ring. In particular, a pyrimidine or triazine ring, which is a six-membered monocyclic ring, is preferable because it achieves a low refractive index, high stability to carriers and excitation, and high reliability compared with a fused aromatic ring.

An organic compound represented by General Formula (G0) below can be given as an example of the organic compound that includes the π-electron deficient heteroaromatic ring and the trisubstituted silyl group having 3 to 18 carbon atoms and can be used in the light-emitting device of one embodiment of the present invention.

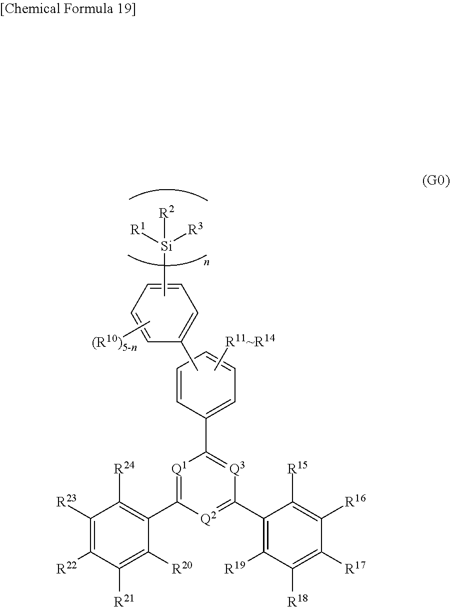

In General Formula (G0), each of Q¹ to Q³ independently represents N or CH; at least two of Q¹ to Q³ represent N; each of R¹ to R³ independently represents an alkyl group having 1 to 6 carbon atoms or a phenyl group; n is an integer greater than or equal to 1 and less than or equal to 5; each of R¹⁰ to R²⁴ independently represents hydrogen (including deuterium), an alkyl group having 1 to 6 carbon atoms, a cycloalkyl group having 3 to 10 carbon atoms, a trisubstituted silyl group having 3 to 18 carbon atoms, a substituted or unsubstituted aryl group having 6 to 30 carbon atoms, or a substituted or unsubstituted heteroaryl group having 3 to 30 carbon atoms; when n is greater than or equal to 2, a plurality of R¹'s may be the same or different from each other, a plurality of R²'s may be the same or different from each other, and a plurality of R³'s may be the same or different from each other; and when 5-n is greater than or equal to 2, a plurality of R¹⁰'s may be the same or different from each other.

Specific examples of the substituent that can be used in the organic compound represented by General Formula (G0) are similar to those described in Embodiment 1 and thus are not described here.

The organic compound represented by any of General Formulae (G1) to (G5) described in Embodiment 1 can be used as an example of the organic compound that includes the π-electron deficient heteroaromatic ring and the trisubstituted silyl group having 3 to 18 carbon atoms and can be used in the light-emitting device of one embodiment of the present invention.

In the case where the organic compound including the π-electron deficient heteroaromatic ring and the trisubstituted silyl group having 3 to 18 carbon atoms is used for a mixed layer in combination with a metal complex, the metal complex is particularly preferably an organic complex including an alkali metal. Examples of the organic complex including an alkali metal include 8-quinolinolato-lithium (abbreviation: Liq), 8-quinolinolato-sodium (abbreviation: Naq), and 8-quinolinolato-potassium (abbreviation: Kq), which are represented by structural formulae below, and derivatives thereof. The organic complex preferably includes an alkyl group such as a methyl group, for example, in which case the refractive index can be reduced. Such a substance is preferably included in a second electron-transport layer 114-2, in which case electron injection from the second electrode 102 can be facilitated and the electron-transport property of the second electron-transport layer 114-2 can be controlled.

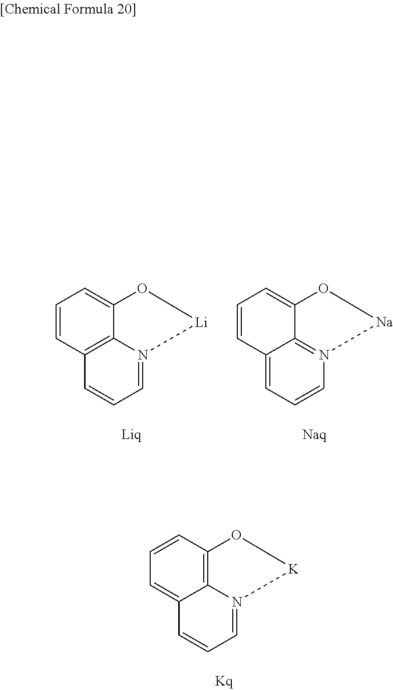

In the case where a mixed layer including the metal complex and the organic compound including the π-electron deficient heteroaromatic ring and the trisubstituted silyl group having 3 to 18 carbon atoms is used as an electron-transport layer, the electron-transport layer is preferably in contact with the cathode. Alternatively, an electron-injection layer preferably has a thickness less than or equal to 5 nm when provided between the electron-transport layer and the cathode. Thus, the effect of the electron-transport property can be enhanced.

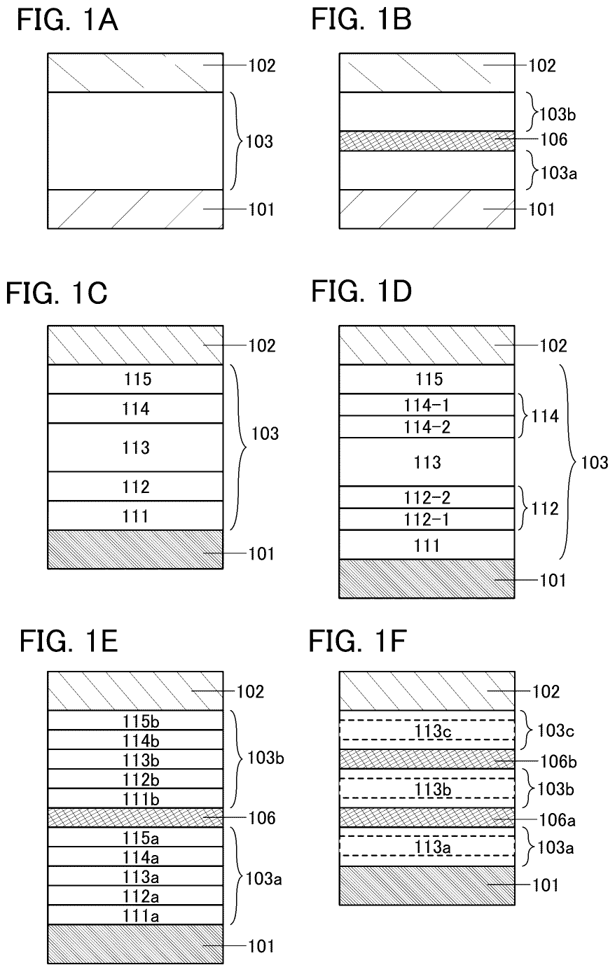

Next, structure examples of a light-emitting device of one embodiment of the present invention is described with reference to FIGS. 1A to 1F.

### <<Basic Structure of Light-Emitting Device>>

A basic structure of a light-emitting device is described. FIG. 1A illustrates a light-emitting device including, between a pair of electrodes, an EL layer including a light-emitting layer. Specifically, an EL layer 103 is located between a first electrode 101 and a second electrode 102.

FIG. 1B illustrates a light-emitting device that has a stacked-layer structure (tandem structure) in which a plurality of EL layers (two EL layers 103a and 103b in FIG. 1B) are provided between a pair of electrodes and a charge-generation layer 106 is provided between the EL layers. A light-emitting device having a tandem structure enables manufacturing of a display device that has high efficiency without changing the amount of current.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when voltage is applied in FIG. 1B such that the potential of the first electrode 101 can be higher than that of the second electrode 102, electrons are injected into the EL layer 103a from the charge-generation layer 106 and holes are injected into the EL layer 103b from the charge-generation layer 106.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

FIG. 1C illustrates a stacked-layer structure of the EL layer 103 in the light-emitting device of one embodiment of the present invention. In this case, the first electrode 101 is regarded as functioning as an anode, and the second electrode 102 is regarded as functioning as a cathode. The EL layer 103 has a structure in which a hole-injection layer 111, a hole-transport layer 112, the light-emitting layer 113, an electron-transport layer 114, and an electron-injection layer 115 are stacked in this order over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure in which a plurality of light-emitting layers that emit light of the same color are stacked can extend driving lifetime; in other words, the structure can achieve higher reliability than a single-layer structure in some cases. In the case where a plurality of EL layers are provided as in the tandem structure illustrated in FIG. 1B, the layers in each EL layer are sequentially stacked from the anode side as described above. When the first electrode 101 is the cathode and the second electrode 102 is the anode, the stacking order of the layers in the EL layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the EL layers (103, 103a, and 103b) contains an appropriate combination of a light-emitting substance and a plurality of substances, so that fluorescent light of a desired color or phosphorescent light of a desired color can be obtained. The plurality of EL layers (103a and 103b) in FIG. 1B may exhibit their respective emission colors. In that case, the light-emitting substances and other substances can be different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 1C. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is k, the optical path length between the first electrode 101 and the second electrode 102 (the product of the thickness and the refractive index) is preferably adjusted to be mλ/2 (m is an integer of 1 or more) or close to mλ/2.

To amplify light obtained from the light-emitting layer 113 at a desired wavelength (λ), it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, the above effect can be sufficiently obtained wherever the reflective regions may be set in the first electrode 101 and the second electrode 102. Furthermore, the optical path length between the first electrode 101 and the light-emitting layer is, to be exact, the optical path length between the reflective region in the first electrode 101 and the light-emitting region in the light-emitting layer. However, it is difficult to precisely determine the reflective region in the first electrode 101 and the light-emitting region in the light-emitting layer; thus, it is assumed that the above effect can be sufficiently obtained wherever the reflective region and the light-emitting region may be set in the first electrode 101 and the light-emitting layer, respectively.

FIG. 1D illustrates a modification example of the stacked-layer structure illustrated in FIG. 1C. Also in this case, the first electrode 101 is regarded as functioning as an anode, and the second electrode 102 is regarded as functioning as a cathode. In this modification example, the hole-transport layer 112 and the electron-transport layer 114 each have a stacked-layer structure of two layers. In other words, the EL layer 103 has a structure in which a hole-injection layer 111, a first hole-transport layer 112-1, a second hole-transport layer 112-2, a light-emitting layer 113, a second electron-transport layer 114-2, a first electron-transport layer 114-1, and an electron-injection layer 115 are stacked in this order over the first electrode 101. Note that the light-emitting layer 113 is positioned between the first electrode 101 and the second electrode 102. The first hole-transport layer 112-1 is positioned between the first electrode 101 and the light-emitting layer 113. The first electron-transport layer 114-1 is positioned between the light-emitting layer 113 and the second electrode 102. The hole-injection layer 111 is positioned between the first electrode 101 and the hole-transport layer 112. The electron-injection layer 115 is positioned between the electron-transport layer 114 and the second electrode 102. The second hole-transport layer 112-2 is positioned between the first hole-transport layer 112-1 and the light-emitting layer 113. In other words, the second electron-transport layer 114-2 is positioned between the light-emitting layer 113 and the first electron-transport layer 114-1. In the case where the EL layer 103 has such a stacked-layer structure, one or more of the electron-injection layer 115, the electron-transport layer 114, and the light-emitting layer 113 preferably contains the organic compound represented by any of General Formulae (G1) to (G5). In particular, the electron-transport layer 114 preferably contains the organic compound represented by any of General Formulae (G1) to (G5), in which case higher efficiency and lower driving voltage of the light-emitting device can be expected.

The second hole-transport layer 112-2 is provided to prevent passing of electrons from the light-emitting layer 113 to the first electrode 101 side, for example. Accordingly, the second hole-transport layer 112-2 can also be referred to as an electron-blocking layer. The second electron-transport layer 114-2 is provided to prevent passing of holes from the light-emitting layer 113 to the second electrode 102 side, for example. Accordingly, the second electron-transport layer 114-2 can also be referred to as a hole-blocking layer. The organic compound represented by any of General Formulae (G1) to (G5) can be used in the second electron-transport layer 114-2 in contact with the light-emitting layer 113. In particular, the first electron-transport layer 114-1 preferably contains the organic compound represented by any of General Formulae (G1) to (G5), in which case higher efficiency and lower driving voltage of the light-emitting device can be expected.

The light-emitting device illustrated in FIG. 1E is a light-emitting device having a tandem structure. Owing to a microcavity structure of the light-emitting device, light (monochromatic light) with different wavelengths from the EL layers (103a and 103b) can be extracted. It is therefore unnecessary to separately form EL layers for obtaining a plurality of emission colors (e.g., R, G, and B). Thus, high resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 1F is an example of the light-emitting device having the tandem structure illustrated in FIG. 1B, and includes three EL layers (103a, 103b, and 103c) stacked with charge-generation layers (106a and 106b) positioned therebetween, as illustrated in FIG. 1F. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light, or the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity of 1×10⁻² Ωcm or less.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity of 1×10⁻² Ωcm or less.

### <<Specific Structure of Light-Emitting Device>>

Next, a specific structure of the light-emitting device of one embodiment of the present invention will be described. Here, the description is made using FIG. 1E illustrating the tandem structure. Note that the structure of the EL layer applies also to the structure of the light-emitting devices having a single structure in FIGS. 1A and 1C. When the light-emitting device in FIG. 1E has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for the first electrode 101 and the second electrode 102, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use a Group 1 element or a Group 2 element in the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device in FIG. 1E, when the first electrode 101 is the anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

### <Hole-Injection Layer>

The hole-injection layers (111, 111a, and 111b) inject holes from the first electrode 101 serving as the anode and the charge-generation layers (106, 106a, and 106b) to the EL layers (103, 103a, and 103b) and contain an organic acceptor material and a material having a high hole-injection property.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound including an electron-withdrawing group (e.g., a halogen group or a cyano group), such as a quinodimethane derivative, a chloranil derivative, and a hexaazatriphenylene derivative, can be used. Examples of the organic acceptor material include 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), and 2-(7-dicyanomethylen-1,3,4,5,6,8,9,10-octafluoro-7H-pyren-2-ylidene)malononitrile. Note that among organic acceptor materials, a compound in which electron-withdrawing groups are bonded to fused aromatic rings each having a plurality of heteroatoms, such as HAT-CN, is particularly preferable because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3, 5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 of the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among the above oxides, molybdenum oxide is preferable because it is stable in atmospheric air, has a low hygroscopic property, and is easily handled. Other examples include a perylenetetracarboxylic acid derivative such as diquinoxalino[2,3-a:2′,3′-c]phenazine (abbreviation: HATNA), 2,3,8,9,14,15-hexafluorodiquinoxalino[2,3-a:2′,3′-c]phenazine (abbreviation: HATNA-F6), 3,4,9,10-perylenetetracarboxylic diimide (abbreviation: PTCDI), or 3,4,9,10-perylenetetracarboxyl-bis-benzimidazole (abbreviation: PTCBI); (C₆₀-Iₕ) [5,6]fullerene (abbreviation: C₆₀); (C₇₀-D₅ₕ) [5,6]fullerene (abbreviation: C₇₀); an organic compound such as phthalocyanine (abbreviation: H₂Pc); and a metal phthalocyanine containing copper, zinc, cobalt, iron, chromium, nickel, or the like or a derivative thereof, such as copper phthalocyanine (abbreviation: CuPc), zinc phthalocyanine (abbreviation: ZnPc), cobalt phthalocyanine (abbreviation: CoPc), iron phthalocyanine (abbreviation: FePc), tin phthalocyanine (abbreviation: SnPc), tin oxide phthalocyanine (abbreviation: SnOPc), titanium oxide phthalocyanine (abbreviation: TiOPc), or vanadium oxide phthalocyanine (abbreviation: VOPc). A phthalocyanine-based metal complex such as CuPc or ZnPc and 2,3,8,9,14,15-hexafluorodiquinoxalino[2,3-a:2′,3′-c]phenazine are especially preferable. Among these materials, CuPc and ZnPc are preferable because they are inexpensive and have favorable characteristics. Using ZnPc, which has a low diffusion coefficient with respect to silicon, reduces the probability that metal diffusion to a semiconductor adversely affects the semiconductor characteristics; accordingly, ZnPc is particularly suitable for a display device manufactured using a silicon semiconductor.

Other examples are aromatic amine compounds, which are low-molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples include high-molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N-phenylamino}phenyl)methacrylamide](abbreviation: PTPDMA), and poly[N,N′-bis(4-butylphenyl)-N,N′-bis(phenyl)benzidine](abbreviation: Poly-TPD). Alternatively, it is possible to use a high-molecular compound to which acid is added, such as poly(3,4-ethylenedioxythiophene)/polystyrenesulfonic acid (abbreviation: PEDOT/PSS) or polyaniline/polystyrenesulfonic acid (abbreviation: PAni/PSS), for example.

As the material having a high hole-injection property, a mixed material containing a hole-transport material and the above-described organic acceptor material (electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that other substances can also be used as long as the substances have hole-transport properties higher than electron-transport properties.

As the hole-transport material, materials having a high hole-transport property, such as a compound including a π-electron rich heteroaromatic ring (e.g., a carbazole derivative, a furan derivative, and a thiophene derivative) and an aromatic amine (an organic compound including an aromatic amine skeleton), are preferable.

Examples of the carbazole derivative (an organic compound including a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), 9-(2-naphthyl)-9′-phenyl-9H,9′H-3,3′-bi-9H-carbazole (abbreviation: βNCCP), 9-(3-biphenyl)-9′-(2-naphthyl)-3,3′-bi-9H-carbazole (abbreviation: βNCCmBP), 9-(4-biphenyl)-9′-(2-naphthyl)-3,3′-bi-9H-carbazole (abbreviation: βNCCBP), and 9,9′-di-2-naphthyl-3,3′-9H,9′H-bicarbazole (abbreviation: BisPNCz).

Specific examples of the aromatic amine having a carbazolyl group include 4-phenyl-4′-(9-phenyl-9H-carbazol-3-yl)triphenylamine (abbreviation: PCBA1BP), N-(biphenyl-4-yl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-(1,1′:3′,1″-terphenyl-4-yl)-9,9-dimethyl-9H-fluoren-2-amine, N-[4-(9-phenyl-9H-carbazol-3-yl)phenyl]-N-(1,1′:4′,1″-terphenyl-4-yl)-9,9-dimethyl-9H-fluoren-2-amine, N-[4-(9-phenyl-9H-carbazol-3-yl)phenyl]-N-(1,1′:3′,1″-terphenyl-4-yl)-9,9-dimethyl-9H-fluoren-4-amine, N-[4-(9-phenyl-9H-carbazol-3-yl)phenyl]-N-(1,1′:4′,1″-terphenyl-4-yl)-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), PCzPCA1, PCzPCA2, PCzPCN1, 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), N-(9,9-spirobi[9H-fluoren]-2-yl)-N,9-diphenylcarbazol-3-amine (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazol-3-yl)phenyl]phenanthrene (abbreviation: PCPPn), 3-[4-(1-naphthyl)phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), and 9-[3-(triphenylsilyl)phenyl]-3,9′-bi-9H-carbazole (abbreviation: PSiCzCz).

Specific examples of the furan derivative (an organic compound including a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound including a thiophene ring) include 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N′-phenyl-N′-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), N-(9,9-spirobi[9H-fluoren]-2-yl)-N,N,N′-triphenyl-1,4-phenyldiamine (abbreviation: DPASF), N,N′-diphenyl-N,N′-bis(4-diphenylaminophenyl)spirobi[9H-fluorene]-2,7-diamine (abbreviation: DPA2SF), 4,4′,4″-tris[N-(1-naphthyl)-N-phenylamino]triphenylamine (abbreviation: 1′-TNATA), TDATA, 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), DPAB, DNTPD, DPA3B, N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other than the above, PVK, PVTPA, PTPDMA, Poly-TPD, or the like that is a high-molecular compound (e.g., an oligomer, a dendrimer, or a polymer) can be used as the hole-transport material. Alternatively, a high-molecular compound to which acid is added, such as PEDOT/PSS or PAni/PSS can be used, for example.

Note that the hole-transport material is not limited to the above examples, and any of a variety of known materials may be used alone or in combination as the hole-transport material.

The hole-injection layers (111, 111a, and 111b) can be formed by any of known film formation methods such as a vacuum evaporation method.

### <Hole-Transport Layer>

The hole-transport layers (112, 112a, and 112b) transport the holes, which are injected from the first electrode 101 by the hole-injection layers (111, 111a, and 111b), to the light-emitting layers (113, 113a, and 113b). Note that the hole-transport layers (112, 112a, and 112b) each include a hole-transport material. Thus, the hole-transport layers (112, 112a, and 112b) can be formed using any of the hole-transport materials that can be used for the hole-injection layers (111, 111a, and 111b).

Note that in the light-emitting device of one embodiment of the present invention, the organic compound used for the hole-transport layers (112, 112a, and 112b) can also be used for the light-emitting layers (113, 113a, and 113b). The same organic compound is preferably used for the hole-transport layers (112, 112a, and 112b) and the light-emitting layers (113, 113a, and 113b), in which case holes can be efficiently transported from the hole-transport layers (112, 112a, and 112b) to the light-emitting layers (113, 113a, and 113b).

### <Light-Emitting Layer>

The light-emitting layers (113, 113a, and 113b) include a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables exhibiting different emission colors (e.g., white light emission obtained by a combination of complementary emission colors). When a plurality of light-emitting layers are provided, the light-emitting layers can exhibit the same color. The structure in which a plurality of light-emitting layers that emit light of the same color are stacked can sometimes achieve higher reliability than a single-layer structure. Furthermore, a stacked-layer structure in which one light-emitting layer contains two or more kinds of light-emitting substances may be employed.

The light-emitting layers (113, 113a, and 113b) may each contain one or more kinds of organic compounds (e.g., a host material) in addition to a light-emitting substance (a guest material).

In the case where a plurality of host materials are used in the light-emitting layers (113, 113a, and 113b), a second host material that is additionally used is preferably a substance having a larger energy gap than those of a known guest material and a first host material. Preferably, the lowest singlet excitation energy level (S₁ level) of the second host material is higher than that of the first host material, and the lowest triplet excitation energy level (T₁ level) of the second host material is higher than that of the guest material. Preferably, the lowest triplet excitation energy level (T₁ level) of the second host material is higher than that of the first host material. With such a structure, an exciplex can be formed by the two kinds of host materials. To form an exciplex efficiently, it is particularly preferable to combine a compound that easily accepts holes (hole-transport material) and a compound that easily accepts electrons (electron-transport material). With the above structure, high efficiency, low voltage, and a long lifetime can be achieved at the same time.

As an organic compound used as the host material (including the first host material and the second host material), organic compounds such as the hole-transport materials usable for the hole-transport layers (112, 112a, and 112b) described above and electron-transport materials usable for electron-transport layers (114, 114a, and 114b) described later can be used as long as they satisfy requirements for the host material used in the light-emitting layer. Another example is an exciplex formed by two or more kinds of organic compounds (the first host material and the second host material). An exciplex whose excited state is formed by two or more kinds of organic compounds has an extremely small energy difference between the S₁ level and the T₁ level and functions as a thermally activated delayed fluorescent (TADF) material capable of converting triplet excitation energy into singlet excitation energy. In an example of a preferable combination of two or more kinds of organic compounds forming an exciplex, one compound of the two or more kinds of organic compounds has a π-electron deficient heteroaromatic ring and the other compound has a π-electron rich heteroaromatic ring. A phosphorescent substance such as an iridium-, rhodium-, or platinum-based organometallic complex or a metal complex may be used as one compound of the combination for forming an exciplex. The organic compound including a trisubstituted silyl group described in Embodiment 1 and this embodiment has an electron-transport property and thus can be used as the host material.

There is no particular limitation on the light-emitting substances that can be used for the light-emitting layers (113, 113a, and 113b), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances that emit fluorescent light (fluorescent substances) can be given as examples of the light-emitting substance that converts singlet excitation energy into light emission and can be used in the light-emitting layers (113, 113a, and 113b): a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine](abbreviation: 1,6BnfAPrn), N,N′-(pyrene-1,6-diyl)bis[(N-phenylbenzo[b]naphtho[1,2-d]furan)-8-amine](abbreviation: 1,6BnfAPrn-02), and N,N′-(pyrene-1,6-diyl)bis[(6,N-diphenylbenzo[b]naphtho[1,2-d]furan)-8-amine](abbreviation: 1,6BnfAPrn-03).

In addition, it is possible to use, for example, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N″-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), and N-[4-(9,10-diphenyl-2-anthryl)phenyl]-N,N′,N′-triphenyl-1,4-phenylenediamine (abbreviation: 2DPAPPA).

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABP}hA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), 1,6BnfAPrn-03, N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), or 3,10-bis[N-(dibenzofuran-3-yl)-N-phenylamino]naphtho[2,3-b;6,7-b′]bisbenzofuran (abbreviation: 3,10FrA2Nbf(IV)-02). In particular, a pyrenediamine compound such as 1,6FLPAPrn, 1,6mMemFLPAPrn, or 1,6BnfAPrn-03 can be used, for example.

A fused heteroaromatic compound including nitrogen and boron, especially a compound having a diaza-boranaphtho-anthracene skeleton, emits high color purity blue light with a narrow emission spectrum and can thus be suitably used. Examples of the compound include 5,9-diphenyl-5H,9H-[1,4]benzazaborino[2,3,4-k/]phenazaborine (abbreviation: DABNA-1), 9-(biphenyl-3-yl)-N,N,5,11-tetraphenyl-5H,9H-[1,4]benzazaborino[2,3,4-kl]phenazaborin-3-amine (abbreviation: DABNA-2), 2,12-di(tert-butyl)-5,9-di(4-tert-butylphenyl)-N,N-diphenyl-5H,9H-[1,4]benzazaborino[2,3,4-kl]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-kl]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-kl][1,4]benzazaborino[4′,3′,2′:4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, a compound having an indole skeleton, such as 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-G), 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-Y), or the like can be suitably used.

<<Light-Emitting Substance that Converts Triplet Excitation Energy into Light Emission>>

Examples of the light-emitting substance that converts triplet excitation energy into light and that can be used in the light-emitting layer 113 include substances that emit phosphorescent light (phosphorescent substances) and TADF materials that exhibit thermally activated delayed fluorescence.

A phosphorescent substance is a compound that emits phosphorescent light but does not emit fluorescent light at a temperature higher than or equal to a low temperature (e.g., 77 K) and lower than or equal to room temperature (i.e., higher than or equal to 77 K and lower than or equal to 313 K). The phosphorescent substance preferably includes a metal element with large spin-orbit interaction, and can be an organometallic complex, a metal complex (platinum complex), or a rare earth metal complex, for example. Specifically, the phosphorescent substance preferably includes a transition metal element. It is preferable that the phosphorescent substance include a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (Peaking at Wavelength from 450 nm to 570 nm: Blue or Green)>>

As examples of a phosphorescent substance which emits blue or green light and whose emission spectrum has a peak wavelength higher than or equal to 450 nm and lower than or equal to 570 nm, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImIr); organometallic complexes having a benzimizazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), bis[2-(4′,6′-difluorophenyl)pyridinato-N,C^{2′}]iridium(III) acetylacetonate (abbreviation: FIr(acac)); and platinum complexes such as (2-{3-[3-(3,5-di-tert-butylphenyl)benzimidazol-1-yl-2-ylidene-κC²]phenoxy-κC²}-9-(4-tert-butyl-2-pyridinyl-κN)carbazole-2,1-diyl-κC¹)platinum(II) (abbreviation: PtON-TBBI). Alternatively, a compound obtained by substituting deuterium for part of hydrogen in any of these compounds can also be used.

<<Phosphorescent Substance (Peaking at Wavelength from 495 nm to 590 nm: Green or Yellow)>>

As examples of a phosphorescent substance which emits green or yellow light and whose emission spectrum has a peak wavelength higher than or equal to 495 nm and lower than or equal to 590 nm, the following substances can be given.

Examples of the phosphorescent substance include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d3-methyl-2-pyridinyl-κN²)phenyl-κC]iridium(III) (abbreviation: Ir(5mppy-d₃)₂(mbfpypy-d₃)), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-(methyl-d₃)-8-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), [2-methyl-8-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]), and tris{2-[5-(methyl-d₃)-4-phenyl-2-pyridinyl-κN]phenyl-κC}iridium(III) (abbreviation: Ir(5m4dppy-d₃)₃); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); organometallic platinum complexes, such as (2-{1-(5-tert-butylbiphenyl-2-yl)-4-[3-tert-butyl-5-(4-phenyl-2-pyridinyl-κN)phenyl-κC⁶]-2-benzimidazolyl-κN³}-4,6-di-tert-butylphenolato-κO)platinum(II) (abbreviation: Pt(tBudppymmtBubiz-tBubp)) and [2-(4-(3,5-di-tert-butylphenyl)-6-{3-[4-(5′-tert-butyl[1,1′:3′,1″-terphenyl]-2′-yl)-2-pyridinyl-κN]phenyl-κC²}-2-pyridinyl-κN)phenolato-κO]platinum(II) (abbreviation: Pt(4tButpppypyp-mmtBup)); and a rare earth metal complex such as tris(acetylacetonato) (monophenanthroline)terbium(III) (abbreviation: [Tb(acac)₃(Phen)]). Alternatively, a compound obtained by substituting deuterium for part of hydrogen in any of these compounds can also be used.

<<Phosphorescent Substance (Peaking at Wavelength from 570 nm to 750 nm: Yellow or Red)>>

As examples of a phosphorescent substance which emits yellow or red light and whose emission spectrum has a peak wavelength higher than or equal to 570 nm and lower than or equal to 750 nm, the following substances can be given.

Examples of the phosphorescent substance include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ2O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC}(2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N, C^{2′})iridium(III) (abbreviation: [Ir(mpq)₂(acac)]), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); organometallic complexes each having a pyridine ring, such as (3,7-diethyl-4,6-nonanedionato-κO⁴,κO⁶)bis[2,4-dimethyl-6-[7-(1-methylethyl)-1-isoquinolinyl-N]phenyl-κC]iridium(III) and (3,7-diethyl-4,6-nonanedionato-κO⁴,κO⁶)bis[2,4-dimethyl-6-[5-(1-methylethyl)-2-quinolinyl-κN]phenyl-κC]iridium(III); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). Alternatively, a compound obtained by substituting deuterium for part of hydrogen in any of these compounds can also be used.

### <<TADF Material>>

Any of materials described below can be used as the TADF material. The TADF material is a material that has a small energy difference between its S₁ and T₁ levels (preferably less than or equal to 0.20 eV), enables up-conversion of a triplet excited state into a singlet excited state (i.e., reverse intersystem crossing) using a little thermal energy, and efficiently exhibits light (fluorescent light) from the singlet excited state. The thermally activated delayed fluorescence is efficiently obtained under the condition where the difference in energy between the triplet excitation energy level and the singlet excitation energy level is greater than or equal to 0.00 eV and less than or equal to 0.20 eV, preferably greater than or equal to 0.00 eV and less than or equal to 0.10 eV. Delayed fluorescent light by the TADF material refers to light emission having a spectrum similar to that of normal fluorescent light and an extremely long lifetime. The lifetime is longer than or equal to 1×10⁻⁶ seconds, or longer than or equal to 1×10⁻³ seconds.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

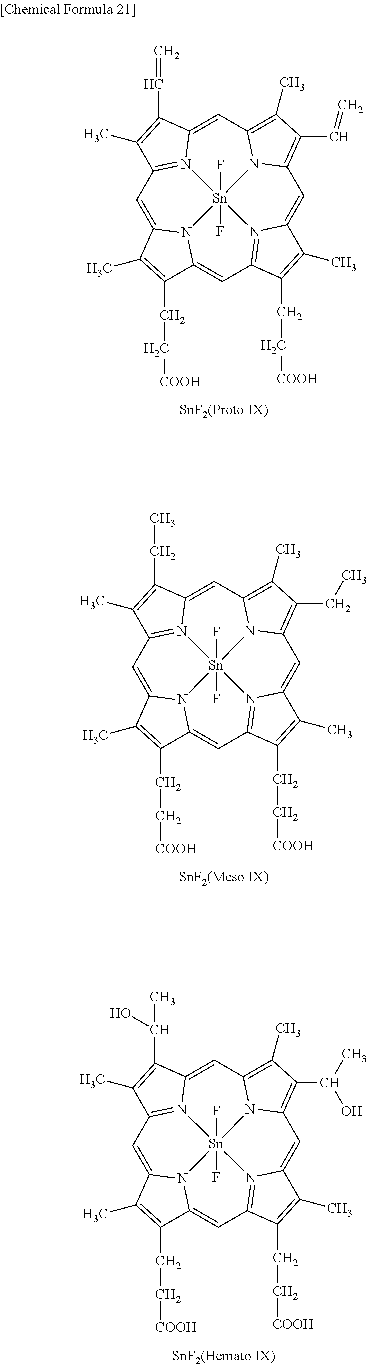

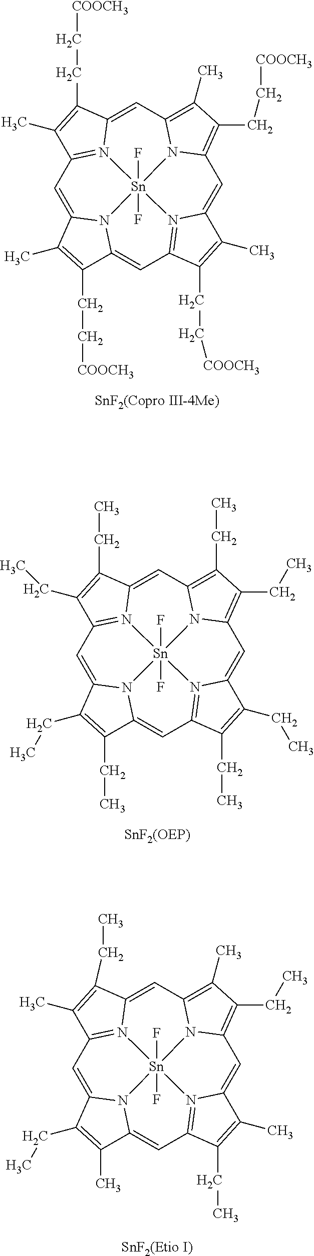

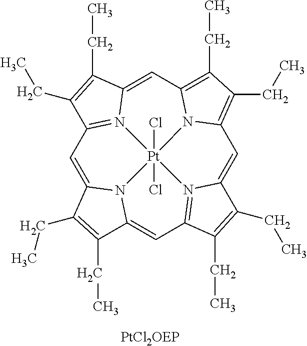

Additionally, a heteroaromatic compound including a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-[3,3′-bi-9H-carbazol]-9-yl)benzofuro[3,2-d]pyrimidine (abbreviation: 4PCCzBfpm), 4-[4-(9′-phenyl-[3,3′-bi-9H-carbazol]-9-yl)phenyl]benzofuro[3,2-d]pyrimidine (abbreviation: 4PCCzPBfpm), or 9-[3-(4,6-diphenyl-1,3,5-triazin-2-yl)phenyl]-9′-phenyl-2,3′-bi-9H-carbazole (abbreviation: mPCCzPTzn-02) may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are enhanced and the energy difference between the singlet excited state and the triplet excited state becomes small. As the TADF material, a TADF material in which the singlet and triplet excited states are in thermal equilibrium (TADF100) may be used. Since such a TADF material enables a short emission lifetime (excitation lifetime), the efficiency of a light-emitting device in a high-luminance region can be less likely to decrease.

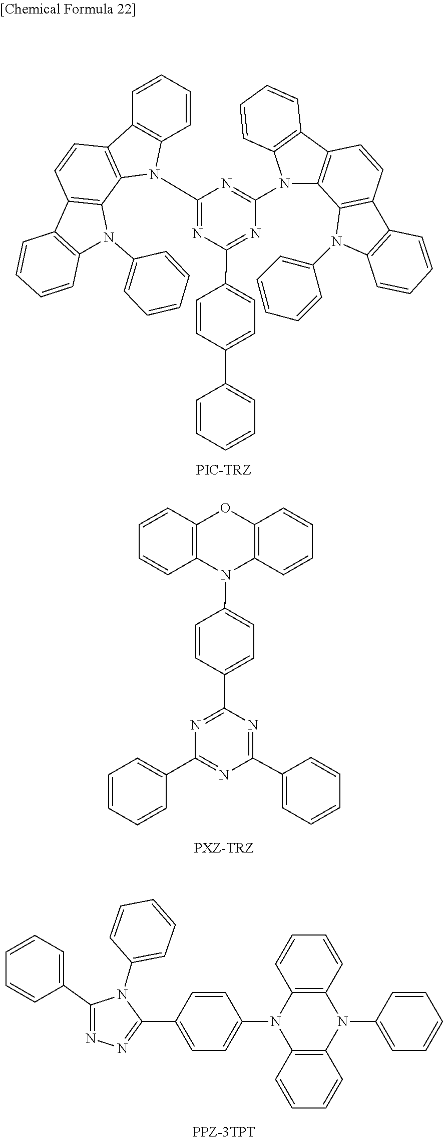 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

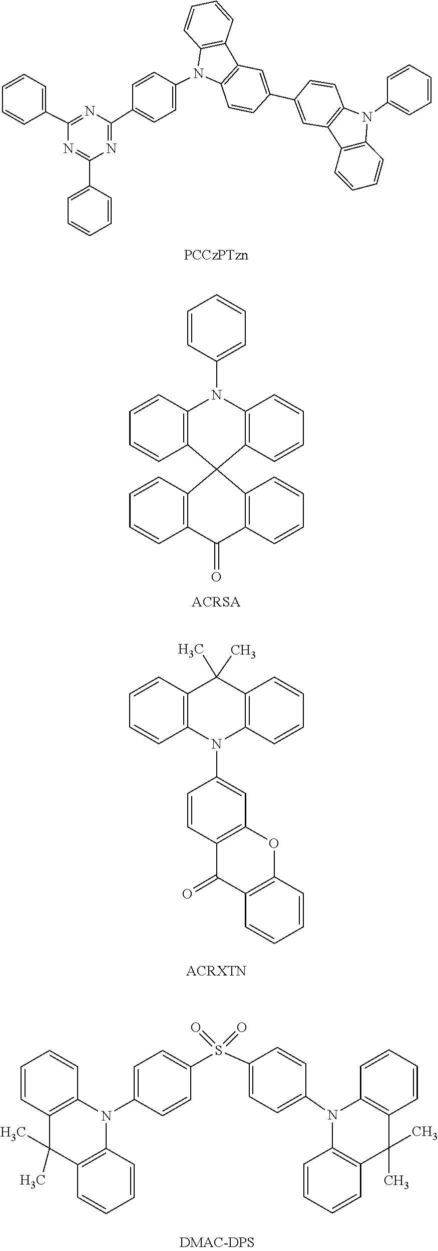 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; O=C1c2ccccc2C2(c3ccccc31)c1ccccc1N(c1ccccc1)c1ccccc12 ; CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21]

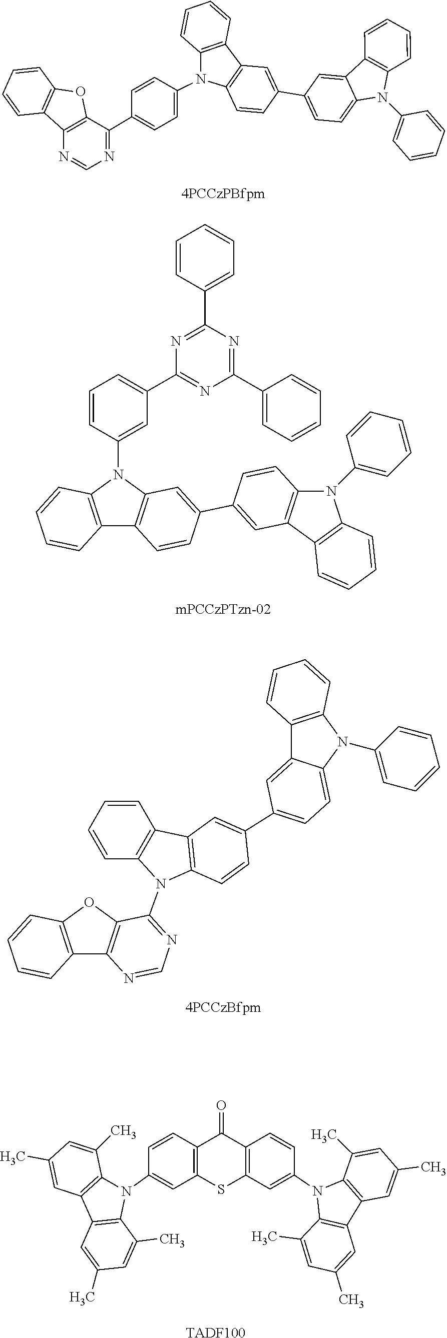

In addition to the above, another example of a material having a function of converting triplet excitation energy into light emission is a nano-structure of a transition metal compound having a perovskite structure. In particular, a nano-structure of a metal halide perovskite material is preferable. The nano-structure is preferably a nanoparticle or a nanorod.

As the organic compound (e.g., the host material) used in combination with the above-described light-emitting substance (guest material) in the light-emitting layers (113, 113a, and 113b), one or more kinds selected from substances having a larger energy gap than the light-emitting substance (guest material) can be used.

### <<Host Material for Fluorescence>>

In the case where the light-emitting substance used in the light-emitting layers (113, 113a, and 113b) is a fluorescent substance, an organic compound (a host material) used in combination with the fluorescent substance is preferably an organic compound that has a high energy level in a singlet excited state and has a low energy level in a triplet excited state or an organic compound having a high fluorescence quantum yield. Therefore, the hole-transport material (described above) and the electron-transport material (described below) shown in this embodiment, for example, can be used as long as they are organic compounds that satisfy such a condition. In addition, the organic compound including a trisubstituted silyl group described in Embodiment 1 and this embodiment can be used.

In terms of a preferable combination with the light-emitting substance (fluorescent substance), examples of the organic compound (host material), some of which overlap the above specific examples, include fused polycyclic aromatic compounds such as an anthracene derivative, a tetracene derivative, a phenanthrene derivative, a pyrene derivative, a chrysene derivative, and a dibenzo[g,p]chrysene derivative.

Specific examples of the organic compound (host material) that is preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), PCPN, 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N″′-octaphenyldibenzo[g,p]chrysene-2,7,10,15-tetraamine (abbreviation: DBC1), CzPA, 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9-(1-naphthyl)-10-(2-naphthyl)anthracene (abbreviation: α,β-ADN), 2-(10-phenylanthracen-9-yl)dibenzofuran, 2-(10-phenyl-9-anthryl)-benzo[b]naphtho[2,3-d]furan (abbreviation: Bnf(II)PhA), 9-(1-naphthyl)-10-[4-(2-naphthyl)phenyl]anthracene (abbreviation: αN-βNPAnth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-10-phenylanthracene (abbreviation: 2αN-QNPhA), 9-(2-naphthyl)-10-[3-(2-naphthyl)phenyl]anthracene (abbreviation: βN-mβNPAnth), 1-{4-[10-(biphenyl-4-yl)-9-anthryl]phenyl}-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri(1-pyrenyl)benzene (abbreviation: TPB3), 5,12-diphenyltetracene, and 5,12-bis(biphenyl-2-yl)tetracene.

### <<Host Material for Phosphorescence>>

In the case where the light-emitting substance used in the light-emitting layers (113, 113a, and 113b) is a phosphorescent substance, an organic compound having triplet excitation energy (an energy difference between a ground state and a triplet excited state) which is higher than that of the light-emitting substance is preferably selected as the organic compound (host material) used in combination with the phosphorescent substance. Note that when a plurality of organic compounds (e.g., a first host material and a second host material (or an assist material)) are used in combination with a light-emitting substance so that an exciplex is formed, the plurality of organic compounds are preferably mixed with the phosphorescent substance. In addition, the organic compound including a trisubstituted silyl group described in Embodiment 1 and this embodiment can be used.

With such a structure, light emission can be efficiently obtained by exciplex-triplet energy transfer (ExTET), which is energy transfer from an exciplex to a light-emitting substance. Note that a combination of the plurality of organic compounds that easily forms an exciplex is preferable, and it is particularly preferable to combine a compound that easily accepts holes (hole-transport material) and a compound that easily accepts electrons (electron-transport material).

In terms of a preferred combination with the light-emitting substance (phosphorescent substance), examples of the organic compounds (the host material and the assist material), some of which are mentioned in the above specific examples, include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- or aluminum-based metal complexes.

Specific examples of the aromatic amine and the carbazole derivative, which are organic compounds having a high hole-transport property among the above-described organic compounds, are the same as the specific examples of the hole-transport materials described above, and those materials are preferable as the host material.

Among the above organic compounds, specific examples of the dibenzothiophene derivative and the dibenzofuran derivative, which are organic compounds having a high hole-transport property, are mmDBFFLBi-II, DBF3P-II, DBT3P-II, DBTFLP-III, DBTFLP-IV, and 4-[3-(triphenylen-2-yl)phenyl]dibenzothiophene (abbreviation: mDBTPTp-II), and these materials are each preferable as a host material.

Other examples of preferable host materials include metal complexes having an oxazole-based or thiazole-based ligand, such as bis[2-(2-benzoxazolyl)phenolato]zinc(II) (abbreviation: ZnPBO) and bis[2-(2-benzothiazolyl)phenolato]zinc(II) (abbreviation: ZnBTZ).

Among the above organic compounds, specific examples of the oxadiazole derivative, the triazole derivative, the benzimidazole derivative, the quinoxaline derivative, the dibenzoquinoxaline derivative, the quinazoline derivative, and the phenanthroline derivative, which are organic compounds having a high electron-transport property, include an organic compound including a heteroaromatic ring having an azole ring such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound including a heteroaromatic ring having a phenanthroline ring such as bathophenanthroline (abbreviation: BPhen), bathocuproine (abbreviation: BCP), 2,9-di(naphthalen-2-yl)-4,7-diphenyl-1,10-phenanthroline (abbreviation: NBPhen), or 2,2′-(1,3-phenylene)bis(9-phenyl-1,10-phenanthroline) (abbreviation: mPPhen2P); and an organic compound including a heteroaromatic ring having a dibenzoquinoxaline ring such as 2-[3-(dibenzothiophen-4-yl)phenyl]dibenzo[f;h]quinoxaline (abbreviation: 2mDBTPDBq-II), 2-[3′-(dibenzothiophen-4-yl)biphenyl-3-yl]dibenzo[f;h]quinoxaline (abbreviation: 2mDBTBPDBq-II), 2-[3′-(9H-carbazol-9-yl)biphenyl-3-yl]dibenzo[f;h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), or 2-[4′-(9-phenyl-9H-carbazol-3-yl)-3,1′-biphenyl-1-yl]dibenzo[f,h]quinoxaline (abbreviation: 2mpPCBPDBq). These organic compounds are preferable as the host material.

Among the above organic compounds, specific examples of the pyridine derivative, the diazine derivative (e.g., the pyrimidine derivative, the pyrazine derivative, and the pyridazine derivative), the triazine derivative, and the furodiazine derivative, which are organic compounds having a high electron-transport property, include organic compounds including a heteroaromatic ring having a diazine ring such as 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), PCCzPTzn, mPCCzPTzn-02, 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 11-[3′-(dibenzothiophen-4-yl)biphenyl-3-yl]phenanthro[9′,10′:4,5]furo[2,3-b]pyrazine (abbreviation: 11mDBtBPPnfpr), 11-[3′-(dibenzothiophen-4-yl)biphenyl-4-yl]phenanthro[9′,10′:4,5]furo[2,3-b]pyrazine, 11-[(3′-9H-carbazol-9-yl)biphenyl-3-yl]phenanthro[9′,10′:4,5]furo[2,3-b]pyrazine, 12-(9′-phenyl-[3,3′-bi-9H-carbazol]-9-yl)phenanthro[9′,10′:4,5]furo[2,3-b]pyrazine (abbreviation: 12PCCzPnfpr), 9-[3′-(9-phenyl-9H-carbazol-3-yl)biphenyl-4-yl]naphtho[1′,2′:4,5]furo[2,3-b]pyrazine (abbreviation: 9pmPCBPNfpr), 9-(9′-phenyl-[3,3′-bi-9H-carbazol]-9-yl)naphtho[1′,2′:4,5]furo[2,3-b]pyrazine (abbreviation: 9PCCzNfpr), 10-(9′-phenyl-[3,3′-bi-9H-carbazol]-9-yl)naphtho[1′,2′:4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′:4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′:4,5]furo[2,3-b]pyrazine (abbreviation: 9mFDBtPNfpr), 9-[3′-(6-phenyldibenzothiophen-4-yl)biphenyl-3-yl]naphtho[1′,2′:4,5]furo[2,3-b]pyrazine (abbreviation: 9mDBtBPNfpr-02), 9-[3-(9′-phenyl-[3,3′-bi-9H-carbazol]-9-yl)phenyl]naphtho[1′,2′:4,5]furo[2,3-b]pyrazine (abbreviation: 9mPCCzPNfpr), 9-[3′-(2,8-diphenyldibenzothiophen-4-yl)biphenyl-3-yl]naphtho[1′,2′:4,5]furo[2,3-b]pyrazine, 11-[3′-(2,8-diphenyldibenzothiophen-4-yl)biphenyl-3-yl]phenanthro[9′,10′:4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-(9,9′-spirobi[9H-fluoren]-2-yl)-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-[8-([1,1′:4′,1″-terphenyl]-4-yl)-1-dibenzofuranyl]-1,3,5-triazine (abbreviation: mBP-TPDBfTzn), 6-(biphenyl-3-yl)-4-[3,5-bis(9H-carbazol-9-yl)phenyl]-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm), and those materials are preferable as the host material.

Among the above organic compounds, specific examples of metal complexes that are organic compounds having a high electron-transport property include zinc- or aluminum-based metal complexes, such as tris(8-quinolinolato)aluminum(III) (abbreviation: Alq₃), tris(4-methyl-8-quinolinolato)aluminum(III) (abbreviation: Almq₃), bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), and bis(8-quinolinolato)zinc(II) (abbreviation: Znq), and metal complexes having a quinoline ring or a benzoquinoline ring. These metal complexes are preferable as the host material.

Moreover, high-molecular compounds such as poly(2,5-pyridinediyl) (abbreviation: PPy), poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) are preferable as the host material.

Furthermore, the following organic compounds with a diazine ring, which have a bipolar property, a high hole-transport property, and a high electron-transport property, can be used as the host material: 9-phenyl-9′-(4-phenyl-2-quinazolinyl)-3,3′-bi-9H-carbazole (abbreviation: PCCzQz), 2mpPCBPDBq, mINc(II)PTzn, 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), and 7-[4-(9-phenyl-9H-carbazol-2-yl)quinazolin-2-yl]-7H-dibenzo[c,g]carbazole (abbreviation: PC-cgDBCzQz).

### <Electron-Transport Layer>

The electron-transport layers (114, 114a, and 114b) transport electrons, which are injected from the second electrode 102 and the charge-generation layers (106, 106a, and 106b) by electron-injection layers (115, 115a, and 115b) described later, to the light-emitting layers (113, 113a, and 113b). The heat resistance of the light-emitting device of one embodiment of the present invention can be improved by including the stacked electron-transport layers. The electron-transport material used in the electron-transport layers (114, 114a, and 114b) is preferably a substance having an electron mobility higher than or equal to 1×10⁻⁶ cm²/Vs in the case where the square root of the electric field strength [V/cm] is 600. Note that any other substance can also be used as long as the substance has an electron-transport property higher than a hole-transport property. The electron-transport layers (114, 114a, and 114b) can function even with a single-layer structure and may have a stacked-layer structure including two or more layers. When a photolithography process is performed over the electron-transport layer including the above-described mixed material, which has heat resistance, an adverse effect of the thermal process on the device characteristics can be reduced.

### <<Electron-Transport Material>>

As the electron-transport material that can be used for the electron-transport layers (114, 114a, and 114b), an organic compound having a high electron-transport property can be used, and for example, a heteroaromatic compound can be used. The term heteroaromatic compound refers to a cyclic compound including at least two different kinds of elements in a ring. Examples of cyclic structures include a three-membered ring, a four-membered ring, a five-membered ring, a six-membered ring, and the like, among which a five-membered ring and a six-membered ring are particularly preferable. The elements included in the heteroaromatic compound are preferably one or more of nitrogen, oxygen, sulfur, and the like in addition to carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used. In addition, the organic compound including a trisubstituted silyl group described in Embodiment 1 and this embodiment can be used.

Note that the electron-transport material can be different from the materials used in the light-emitting layer. Not all excitons formed by recombination of carriers in the light-emitting layer can contribute to light emission and some excitons are diffused into a layer in contact with the light-emitting layer or a layer in the vicinity of the light-emitting layer. In order to avoid this phenomenon, the energy level (the lowest singlet excitation energy level or the lowest triplet excitation energy level) of a material used for the layer in contact with the light-emitting layer or the layer in the vicinity of the light-emitting layer is preferably higher than that of a material used for the light-emitting layer. Thus, when a material different from the material of the light-emitting layer is used as the electron-transport material, a device having high efficiency can be obtained.

The heteroaromatic compound is an organic compound including at least one heteroaromatic ring.

The heteroaromatic ring includes any one of a pyridine ring, a diazine ring, a triazine ring, an azole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having an azole ring includes a heteroaromatic ring having an imidazole ring, a triazole ring, or an oxadiazole ring.

The heteroaromatic ring includes a fused heteroaromatic ring having a fused ring structure. Examples of the fused heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like include a heteroaromatic compound having a heteroaromatic ring, such as a pyridine ring, a diazine ring (a pyrimidine ring, a pyrazine ring, a pyridazine ring, or the like), a triazine ring, or an azole ring. Other examples include a heteroaromatic compound having a bipyridine structure, a heteroaromatic compound having a terpyridine structure, and the like, which are included in examples of a heteroaromatic compound in which pyridine rings are connected.

Examples of the heteroaromatic compound having a fused ring structure partly including the above six-membered ring structure include a heteroaromatic compound having a fused heteroaromatic ring such as a quinoline ring, a benzoquinoline ring, a quinoxaline ring, a dibenzoquinoxaline ring, a phenanthroline ring, a furodiazine ring (including a structure in which an aromatic ring is fused to a furan ring of a furodiazine ring), or a benzimidazole ring.

Specific examples of the heteroaromatic compound having a five-membered ring structure (e.g., an azole ring (including an imidazole ring, a triazole ring, and an oxadiazole ring), an oxazole ring, a thiazole ring, or a benzimidazole ring) are PBD, OXD-7, CO11, TAZ, 3-(4-tert-butylphenyl)-4-(4-ethylphenyl)-5-(4-biphenylyl)-1,2,4-triazole (abbreviation: p-EtTAZ), TPBI, mDBTBIm-II, and BzOs.

Specific examples of the above-described heteroaromatic compound having a six-membered ring structure (including a heteroaromatic ring having a pyridine ring, a diazine ring, a triazine ring, or the like) include: a heteroaromatic compound including a heteroaromatic ring having a pyridine ring, such as 35DCzPPy or TmPyPB; a heteroaromatic compound including a heteroaromatic ring having a triazine ring, such as PCCzPTzn, mPCCzPTzn-02, mINc(II)PTzn, mTpBPTzn, BP-SFTzn, 2,4NP-6PyPPm, PCDBfTzn, mBP-TPDBfTzn, 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6mPnP2Pm, 4,6mDBTP2Pm-II, 4,6mCzP2Pm, 4,6mCzBP2Pm, 6mBP-4Cz2PPm, 6BP-4Cz2PPm, 4-[3-(dibenzothiophen-4-yl)phenyl]-8-(naphthalen-2-yl)-[1]benzofuro[3,2-d]pyrimidine (abbreviation: 8PN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′, 2′: 4,5]furo[3,2-d]pyrimidine (abbreviation: 8mDBtBPNfpm), or 8-([2,2′-binaphthalen]-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a fused heteroaromatic ring.

Other examples include heteroaromatic compounds including a heteroaromatic ring having a diazine (pyrimidine) ring, such as 2,2′-(pyridine-2,6-diyl)bis(4-phenylbenzo[h]quinazoline) (abbreviation: 2,6(P-Bqn)₂Py), 2,2′-([2,2′-bipyridine]-6,6′-diyl)bis(4-phenylbenzo[h]quinazoline) (abbreviation: 6,6′(P-Bqn)₂BPy), 2,2′-(pyridine-2,6-diyl)bis{4-[4-(2-naphthyl)phenyl]-6-phenylpyrimidine}(abbreviation: 2,6(NP-PPm)₂Py), or 6mBP-4Cz2PPm, and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tzn), or 2-[3-(2,6-dimethyl-3-pyridinyl)-5-(9-phenanthryl)phenyl]-4,6-diphenyl-1,3,5-triazine (abbreviation: mPn-mDMePyPTzn).

Specific examples of the above-described heteroaromatic compound having a fused ring structure partly including a six-membered ring structure (the heteroaromatic compound having a fused ring structure) include a heteroaromatic compound having a quinoxaline ring, such as BPhen, BCP, NBPhen, mPPhen2P, 2,6(P-Bqn)2Py, 2mDBTPDBq-II, 2mDBTBPDBq-II, 2mCzBPDBq, 2CzPDBq-III, 7mDBTPDBq-II, 6mDBTPDBq-II, and 2mpPCBPDBq.

For the electron-transport layers (114, 114a, and 114b), any of the metal complexes given below can be used as well as the heteroaromatic compounds described above. Examples of the metal complexes include a metal complex having a quinoline ring or a benzoquinoline ring, such as Alq₃, Almq₃, 8-quinolinolato-lithium (abbreviation: Liq), BAlq, and Znq, and a metal complex having an oxazole ring or a thiazole ring, such as ZnPBO and ZnBTZ.

It is also possible to use high-molecular compounds such as PPy, PF-Py, and PF-BPy as the electron-transport material.

Each of the electron-transport layers (114, 114a, and 114b) is not limited to a single layer and may be a stack of two or more layers each including any of the above substances.

### <Electron-Injection Layer>

The electron-injection layers (115, 115a, and 115b) include a substance having a high electron-injection property. The electron-injection layers (115, 115a, and 115b) are layers for increasing the efficiency of electron injection from the second electrode 102 and are preferably formed using a material whose value of the LUMO level has a small difference (less than or equal to 0.50 eV) from the work function of a material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), 8-quinolinolato-lithium (abbreviation: Liq), 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal or a compound of a rare earth metal, such as erbium fluoride (ErF₃) or ytterbium (Yb), can also be used. It is also possible to use a compound including a 1,3,4,6,7,8-tetrahydro-2H-pyrimido[1,2-a]pyrimidine skeleton, such as 1-(9,9′-spirobi[9H-fluoren]-2-yl)-1,3,4,6,7,8-hexahydro-2H-pyrimido[1,2-a]pyrimidine (abbreviation: 2hppSF), 1,1′-(9,9′-spirobi[9H-fluorene]-2,7-diyl)bis(1,3,4,6,7,8-hexahydro-2H-pyrimido[1,2-a]pyrimidine) (abbreviation: 2,7hpp2SF), or 1,1′-pyridine-2,6-diyl-bis(1,3,4,6,7,8-hexahydro-2H-pyrimido[1,2-a]pyrimidine) (abbreviation: hpp2Py). To form the electron-injection layers (115, 115a, and 115b), two or more of the above materials may be mixed or stacked. electride may also be used for the electron-injection layers (115, 115a, and 115b). Examples of an electride include substances in which electrons are added at high concentration to a calcium oxide-aluminum oxide. Any of the substances for forming the electron-transport layers (114, 114a, and 114b), which are given above, can also be used.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. The organic compound here is preferably a material excellent in transporting the generated electrons; specifically, for example, the above-described electron-transport materials used for the electron-transport layers (114, 114a, and 114b), such as a metal complex and a heteroaromatic compound, can be used. As the electron donor, a substance showing an electron-donating property with respect to an organic compound is preferably used. Specifically, an alkali metal, an alkaline earth metal, and a rare earth metal are preferable, and lithium, cesium, magnesium, calcium, erbium, ytterbium, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

A mixed material in which an organic compound and a metal are mixed may also be used for the electron-injection layers (115, 115a, and 115b). The organic compound used here preferably has a LUMO level higher than or equal to −3.60 eV and lower than or equal to −2.30 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure partly including a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples thereof include Ag, Cu, Al, and In. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one fourth of the wavelength k of light emitted from the light-emitting layer 113b. In that case, the optical path length can be adjusted by changing the thickness of the electron-transport layer 114b or the electron-injection layer 115b.

### <Charge-Generation Layer>

The charge-generation layer 106 has a function of injecting electrons into the EL layer 103a and injecting holes into the EL layer 103b when voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these structures may be stacked. Furthermore, an electron-relay layer may be provided between the p-type layer and the electron-injection buffer layer. Note that forming the charge-generation layer 106 with the use of any of the above materials can inhibit an increase in driving voltage caused by the stack of the EL layers.

In the case where the charge-generation layer 106 is a p-type layer in which an electron acceptor is added to a hole-transport material, which is an organic compound, any of the materials described in this embodiment can be used as the hole-transport material. Examples of the electron acceptor include F₄-TCNQ and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films including the respective materials may be used.

In the case where the charge-generation layer 106 is an electron-injection buffer layer in which an electron donor is added to an electron-transport material, any of the materials described in this embodiment can be used as the electron-transport material. In addition, the organic compound including a trisubstituted silyl group described in Embodiment 1 and this embodiment can be used. As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An alkali metal compound such as Liq may be used. An organic compound such as tetrathianaphthacene may be used as the electron donor. An organic compound including a 1,3,4,6,7,8-tetrahydro-2H-pyrimido[1,2-a]pyrimidine skeleton, such as 2hppSF, 2,7hpp2SF, or hpp2Py may be used as the electron donor. When any of these organic compounds is used as the electron donor, the electron-transport material to be combined with the electron donor is preferably an organic compound including a heteroaromatic ring having a phenanthroline ring, such as BPhen, BCP, NBPhen, or 2,2′-(1,3-phenylene)bis(9-phenyl-1,10-phenanthroline) (abbreviation: mPPhen2P), in which case driving voltage of the light-emitting device can be reduced.

When an electron-relay layer is provided between a p-type layer and an electron-injection buffer layer in the charge-generation layer 106, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer can be higher than or equal to −5.00 eV, further preferably higher than or equal to −5.00 eV and lower than or equal to −3.00 eV. Note that as the substance having an electron-transport property in the electron-relay layer, a phthalocyanine-based material or a metal complex having a metal-oxygen bond and an aromatic ligand is preferably used.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

Although FIG. 1E illustrates the structure in which two EL layers 103 are stacked, three or more EL layers may be stacked with charge-generation layers each provided between two adjacent EL layers.

### <Cap Layer>

Although not illustrated in FIGS. 1A to 1F, a cap layer may be provided over the second electrode 102 of the light-emitting device. For example, a material with a high refractive index can be used for the cap layer. When the cap layer is provided over the second electrode 102, extraction efficiency of light emitted through the second electrode 102 can be improved.

Specific examples of a material that can be used for the cap layer include 5,5′-diphenyl-2,2′-di-5H-[1]benzothieno[3,2-c]carbazole (abbreviation: BisBTc) and DBT3P-II.

### <Substrate>

The light-emitting device described in this embodiment can be formed over a variety of substrates. Note that the type of the substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, and paper or a base material film including a fibrous material.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as an acrylic resin, polypropylene, polyester, polyvinyl fluoride, polyvinyl chloride, polyamide, polyimide, aramid, an epoxy resin, an inorganic vapor deposition film, and paper.

For fabrication of the light-emitting device in this embodiment, a gas phase method such as an evaporation method or a liquid phase method such as a spin coating method or an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers with various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, screen printing (stencil), offset printing (planography), flexography (relief printing), gravure printing, or micro-contact printing), or the like.

In the case where a film formation method such as the coating method or the printing method is employed, a high-molecular compound (e.g., an oligomer, a dendrimer, or a polymer), a middle-molecular compound (a compound between a low-molecular compound and a high-molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

The structures described in this embodiment can be used in appropriate combination with any of the structures described in the other embodiments.

### Embodiment 3

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and laptop personal computers, a monitor of a computer and the like, digital signage, and a large game machine such as a pachinko machine.

### [Display Module]

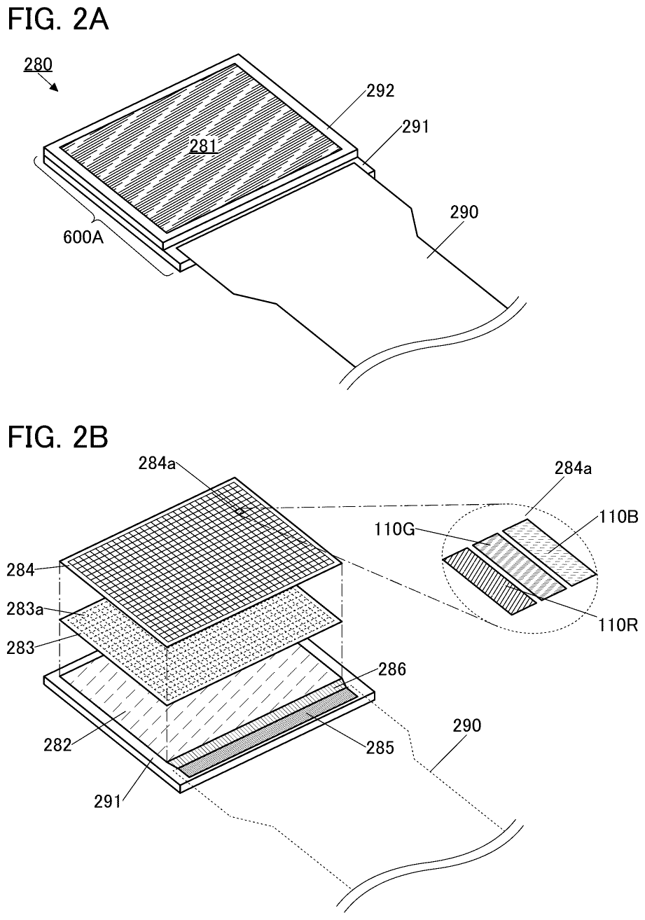

FIG. 2A is a perspective view of a display module 280. The display module 280 includes a display device 600A and an FPC 290. Note that the display device included in the display module 280 is not limited to the display device 600A and may be a display device 600B described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 2B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion over the substrate 291 that does not overlap with the pixel portion 284. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 2B. The pixels 284a can employ any of the structures described in the above embodiments

The pixel circuit portion 283 includes a plurality of pixel circuits 283a arranged periodically.

One pixel circuit 283a is a circuit that controls driving of a plurality of elements included in one pixel 284a. One pixel circuit 283a can be provided with three circuits each of which controls light emission of one light-emitting device. For example, the pixel circuit 283a can include at least one selection transistor, one current control transistor (driving transistor), and a capacitor per light-emitting device. A gate signal is input to a gate of the selection transistor, and a video signal is input to a source or a drain of the selection transistor. Thus, an active-matrix display device is achieved.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high. For example, the aperture ratio of the display portion 281 can be higher than or equal to 40% and lower than 100%, preferably higher than or equal to 50% and lower than or equal to 95%, further preferably higher than or equal to 60% and lower than or equal to 95%. Furthermore, the pixels 284a can be arranged extremely densely and thus the display portion 281 can have significantly high definition. For example, the pixels 284a are preferably arranged in the display portion 281 to give a definition higher than or equal to 2000 ppi, further preferably higher than or equal to 3000 ppi, still further preferably higher than or equal to 5000 ppi, yet still further preferably higher than or equal to 6000 ppi, and lower than or equal to 20000 ppi or lower than or equal to 30000 ppi.

The pixel 284a includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and Y, and four subpixels emitting light of R, G, and B and infrared light (IR).

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion. For example, the display module 280 can be favorably used in a display portion of a wearable electronic appliance, such as a wrist watch.

### [Display Device 600A]

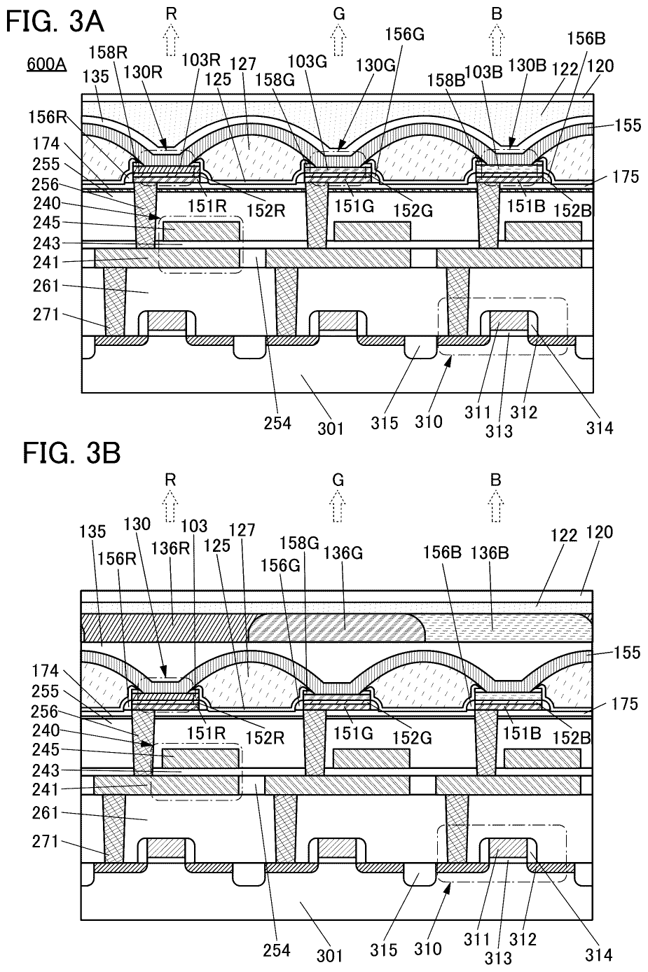

The display device 600A illustrated in FIG. 3A includes a substrate 301, the light-emitting devices 130R, 130G, and 130B, a capacitor 240, and a transistor 310.

The substrate 301 corresponds to the substrate 291 in FIGS. 2A and 2B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B each have a structure as described in Embodiment 2. An insulator is provided in regions between adjacent light-emitting devices. For example, in FIG. 3A, the inorganic insulating layer 125 and the insulating layer 127 over the inorganic insulating layer 125 are provided in those regions.

The light-emitting device 130R includes the first electrode including a conductive layer 151R and a conductive layer 152R, an EL layer 103R over the first electrode, and a common layer 155 over the EL layer 103R. The light-emitting device 130G includes a first electrode including a conductive layer 151G and a conductive layer 152G, an EL layer 103G over the first electrode, and the common layer 155 over the EL layer 103G. The light-emitting device 130B includes a first electrode including a conductive layer 151B and a conductive layer 152B, an EL layer 103B over the first electrode, and the common layer 155 over the EL layer 103B. A sacrificial layer 158R is positioned over the EL layer 103R of the light-emitting device 130R. A sacrificial layer 158G is positioned over the EL layer 103G of the light-emitting device 130G. A sacrificial layer 158B is positioned over the EL layer 103B of the light-emitting device 130B. The common layer 155 shared by the light-emitting devices includes at least a second electrode. The common layer 155 may include an electron-injection layer positioned between the second electrode and each EL layer. For example, the light-emitting device 130R can emit red light, the light-emitting device 130G can emit green light, and the light-emitting device 130B can emit blue light. In this specification and the like, description common to the light-emitting devices 130R, 130G, and 130B is sometimes made using the collective term “light-emitting device 130”. In this specification and the like, description common to the conductive layers 151R, 151G, and 151B is sometimes made using the collective term “conductive layer 151”. In this specification and the like, description common to the conductive layers 152R, 152G, and 152B is sometimes made using the collective term “conductive layer 152”.

In the light-emitting device 130, one of the first electrode and the second electrode functions as an anode and the other functions as a cathode. Hereinafter, description is made on the assumption that the first electrode functions as the anode and the second electrode functions as the cathode unless otherwise specified.

The EL layers included in the light-emitting device 130 are island-shaped and independent of each other on a light-emitting device basis or on an emission color basis. Providing the island-shaped EL layer 103 in each of the light-emitting devices 130 can suppress leakage current between the adjacent light-emitting devices 130 even in a high-resolution display device. This can prevent crosstalk, so that a display device with extremely high contrast can be obtained. Specifically, a display device having high current efficiency at low luminance can be obtained.

In the display device of one embodiment of the present invention, the first electrode (pixel electrode) of the light-emitting device preferably has a stacked-layer structure. For example, in the example shown in FIG. 3B, the first electrode of the light-emitting device 130 is a stack of the conductive layer 151 and the conductive layer 152. In the case where the display device 600A is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display device 600A is of a top-emission type, the higher the visible light reflectance of the pixel electrode is, the higher the efficiency of extraction of the light emitted by the EL layer 103 is. In the case where the pixel electrode functions as the anode, the higher the work function of the pixel electrode is, the easier hole injection into the EL layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

In the case where the conductive layer 151 has high reflectance for visible light, the visible light reflectance of the conductive layer 151 is preferably higher than or equal to 40% and lower than or equal to 100%, or higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

Thus, in the display device 600A of this embodiment, an insulating layer 156 (insulating layers 156R, 156G, and 156B) is formed on the side surfaces of the conductive layers 151 and 152. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display device 600A to be manufactured by a high-yield method and to be accordingly inexpensive. In addition, generation of a defect in the display device 600A can be inhibited, which makes the display device 600A highly reliable. In this specification and the like, description common to the conductive layers 156R, 156G, and 156B is sometimes made using the collective term “conductive layer 156”.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy including an appropriate combination of any of these metals, for example.

For the conductive layer 152, an oxide including one or more selected from indium, tin, zinc, gallium, titanium, aluminum, and silicon can be used. For example, it is preferable to use a conductive oxide including one or more of indium oxide, indium tin oxide, indium zinc oxide, zinc oxide, zinc oxide including gallium, titanium oxide, indium zinc oxide including gallium, indium zinc oxide including aluminum, indium tin oxide including silicon, indium zinc oxide including silicon, and the like. In particular, an indium tin oxide including silicon can be suitably used for the conductive layer 152 because of having a work function of higher than or equal to 4.0 eV, for example.

The conductive layer 151 is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. The top surface of the insulating layer 175 and the top surface of the plug 256 are level with or substantially level with each other. Any of a variety of conductive materials can be used for the plugs.

The protective layer 135 is provided over the light-emitting device 130. A substrate 120 is bonded onto the protective layer 135 with a resin layer 122. The substrate 120 corresponds to the substrate 292 in FIG. 2A.

FIG. 3B illustrates a variation example of the display device 600A illustrated in FIG. 3A. The light-emitting device illustrated in FIG. 3B includes the coloring layers 136R, 136G, and 136B, and each of the light-emitting devices 130 includes a region overlapping with one of the coloring layers 136R, 136G, and 136B. In the display device illustrated in FIG. 3B, the light-emitting device 130 can emit white light, for example. For example, the coloring layer 136R, the coloring layer 136G, and the coloring layer 136B can transmit red light, green light, and blue light, respectively.

### [Display Device 600B]

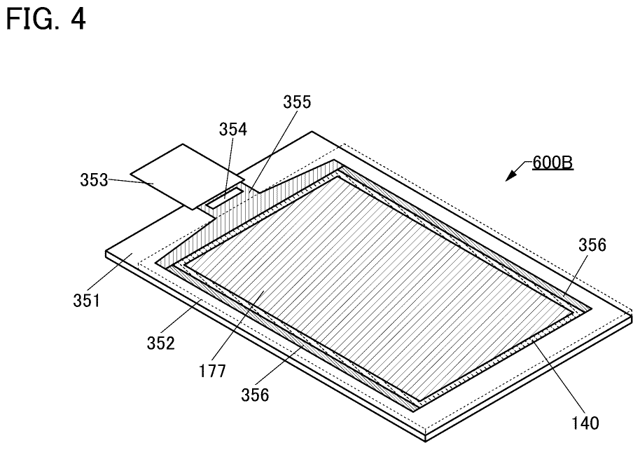

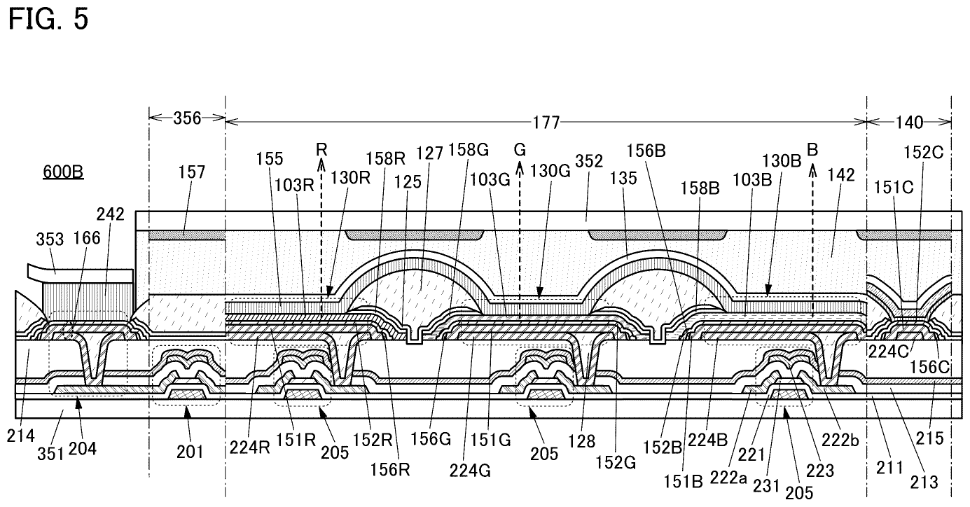

FIG. 4 is a perspective view of the display device 600B, and FIG. 5 is a cross-sectional view of the display device 600B.

In the display device 600B, a substrate 352 and a substrate 351 are bonded to each other. In FIG. 4, the substrate 352 is denoted by a dashed line.

The display device 600B includes the pixel portion 177, the connection portion 140, a circuit 356, a wiring 355, and the like. FIG. 4 shows an example in which an integrated circuit (IC) 354 and an FPC 353 are mounted on the display device 600B. Thus, the structure illustrated in FIG. 4 can be regarded as a display module including the display device 600B, the IC, and the FPC. Here, a display device in which a substrate is equipped with a connector such as an FPC or mounted with an IC is referred to as a display module.

The connection portion 140 is provided outside the pixel portion 177. The connection portion 140 can be provided along one side or a plurality of sides of the pixel portion 177. The number of connection portions 140 may be one or more. FIG. 4 illustrates an example in which the connection portion 140 is provided to surround the four sides of the pixel portion 177. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 4 illustrates an example in which the IC 354 is provided for the substrate 351 by a chip on glass (COG) method, a chip on film (COF) method, or the like. An IC including a scan line driver circuit, a signal line driver circuit, or the like can be used as the IC 354, for example. Note that the display device 600B and the display module are not necessarily provided with an IC. Alternatively, the IC may be mounted on the FPC by a COF method, for example.

FIG. 5 shows an example of cross sections of part of a region including the FPC 353, part of the circuit 356, part of the pixel portion 177, part of the connection portion 140, and part of a region including an end portion of the display device 600B.

The display device 600B shown in FIG. 5 includes a transistor 201, a transistor 205, the light-emitting device 130R that emits red light, the light-emitting device 130G that emits green light, the light-emitting device 130B that emits blue light, and the like between the substrate 351 and the substrate 352.

The stacked-layer structure of each of the light-emitting devices 130R, 130G, and 130B is the same as that shown in FIG. 1A except for the structure of the pixel electrode. The above embodiments can be referred to for the details of the light-emitting devices.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B. Here, the conductive layers 224R, 151R, and 152R can be collectively referred to as the pixel electrode of the light-emitting device 130R; the conductive layers 151R and 152R excluding the conductive layer 224R can also be referred to as the pixel electrode of the light-emitting device 130R. Similarly, the conductive layers 224G, 151G, and 152G can be collectively referred to as the pixel electrode of the light-emitting device 130G; the conductive layers 151G and 152G excluding the conductive layer 224G can also be referred to as the pixel electrode of the light-emitting device 130G. The conductive layers 224B, 151B, and 152B can be collectively referred to as the pixel electrode of the light-emitting device 130B; the conductive layers 151B and 152B excluding the conductive layer 224B can also be referred to as the pixel electrode of the light-emitting device 130B.

The conductive layer 224R is connected to a conductive layer 222b included in the transistor 205 through an opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G, and the insulating layer 156G in the light-emitting device 130G are not described in detail because they are respectively similar to the conductive layers 224R, 151R, and 152R, and the insulating layer 156R in the light-emitting device 130R; the same applies to the conductive layers 224B, 151B, and 152B, and the insulating layer 156B in the light-emitting device 130B.

The conductive layers 224R, 224G, and 224B each have a depressed portion covering the opening provided in the insulating layer 214. A layer 128 is embedded in the depressed portion.

The layer 128 has a function of filling the depressed portions of the conductive layers 224R, 224G, and 224B to obtain planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depressed portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 135 is provided over the light-emitting devices 130R, 130G, and 130B. The protective layer 135 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 5, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided in a frame shape not to overlap with the light-emitting device. Furthermore, the space may be filled with a resin other than the frame-like adhesive layer 142.

FIG. 5 shows an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and a conductive layer 152C obtained by processing the same conductive film as the conductive layers 152R, 152G, and 152B. In the example illustrated in FIG. 5, the insulating layer 156C is provided to include a region overlapping with the side surface of the conductive layer 151C.

The display device 600B has a top-emission structure. Light from the light-emitting device is emitted toward the substrate 352. For the substrate 352, a material with a high visible-light-transmitting property is preferably used. The pixel electrode includes a material that reflects visible light, and a counter electrode (the common electrode 155) includes a material that transmits visible light.

The transistor 201 and the transistor 205 are formed over the substrate 351. These transistors can be fabricated using the same materials in the same steps.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

A material that does not easily allow diffusion of impurities such as water and hydrogen is preferably used for at least one of the insulating layers covering the transistors. This is because such an insulating layer can function as a barrier layer. Such a structure can effectively inhibit diffusion of impurities into the transistors from the outside and improve the reliability of a display device.

An inorganic insulating film is preferably used as each of the insulating layers 211, 213, and 215. As the inorganic insulating film, a silicon nitride film, a silicon oxynitride film, a silicon oxide film, a silicon nitride oxide film, an aluminum oxide film, or an aluminum nitride film can be used, for example. A hafnium oxide film, an yttrium oxide film, a zirconium oxide film, a gallium oxide film, a tantalum oxide film, a magnesium oxide film, a lanthanum oxide film, a cerium oxide film, a neodymium oxide film, or the like may be used. Two or more of the above insulating films may also be stacked.

An organic insulating layer is suitable as the insulating layer 214 functioning as a planarization layer. Examples of materials that can be used for the organic insulating layer include an acrylic resin, a polyimide resin, an epoxy resin, a polyamide resin, a polyimide-amide resin, a siloxane resin, a benzocyclobutene-based resin, a phenol resin, and precursors of these resins. The insulating layer 214 may have a stacked-layer structure of an organic insulating layer and an inorganic insulating layer. The outermost layer of the insulating layer 214 preferably functions as an etching protective layer. This can inhibit formation of a depressed portion in the insulating layer 214 at the time of processing of the conductive layer 224R, 151R, or 152R or the like. Alternatively, a depressed portion may be provided in the insulating layer 214 at the time of processing of the conductive layer 224R, 151R, or 152R or the like.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate. Here, a plurality of layers obtained by processing the same conductive film are shown with the same hatching pattern. The insulating layer 211 is positioned between the conductive layer 221 and the semiconductor layer 231. The insulating layer 213 is positioned between the conductive layer 223 and the semiconductor layer 231.

There is no particular limitation on the structure of the transistors included in the display device of this embodiment. For example, a planar transistor, a staggered transistor, or an inverted staggered transistor can be used. A top-gate transistor or a bottom-gate transistor can be used. Alternatively, gates may be provided above and below a semiconductor layer where a channel is formed.

The structure in which the semiconductor layer where a channel is formed is provided between two gates is employed for each of the transistors 201 and 205. The two gates may be connected to each other and supplied with the same signal to drive the transistor. Alternatively, the threshold voltage of the transistor may be controlled by applying a potential for controlling the threshold voltage to one of the two gates and a potential for driving to the other of the two gates.

There is no particular limitation on the crystallinity of a semiconductor material used for the transistors, and either an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case degradation of transistor characteristics can be inhibited.

The semiconductor layer of the transistor preferably includes a metal oxide. That is, a transistor including a metal oxide in its channel formation region (hereinafter referred to as an OS transistor) is preferably used in the display device of this embodiment.

Examples of an oxide semiconductor having crystallinity include a c-axis-aligned crystalline oxide semiconductor (CAAC-OS) and a nanocrystalline oxide semiconductor (nc-OS).

Alternatively, a transistor including silicon in its channel formation region (a Si transistor) may be used. Examples of silicon include single crystal silicon, polycrystalline silicon, and amorphous silicon. In particular, a transistor including low-temperature polysilicon (LTPS) in its semiconductor layer (hereinafter also referred to as an LTPS transistor) can be used. The LTPS transistor has high field-effect mobility and excellent frequency characteristics.

With the use of Si transistors such as LTPS transistors, a circuit required to be driven at a high frequency (e.g., a source driver circuit) can be formed on the same substrate as the display portion. This allows simplification of an external circuit mounted on the display device and a reduction in costs of parts and mounting costs.

An OS transistor has much higher field-effect mobility than a transistor including amorphous silicon. In addition, the OS transistor has an extremely low leakage current between a source and a drain in an off state, and charge accumulated in a capacitor that is connected in series to the transistor can be held for a long period. Furthermore, the use of an OS transistor can reduce the power consumption of the display device.

To increase the luminance of the light-emitting device included in the pixel circuit, the amount of current fed through the light-emitting device needs to be increased. To increase the current amount, the source-drain voltage of a driving transistor included in the pixel circuit needs to be increased. An OS transistor has a higher breakdown voltage between a source and a drain than a Si transistor; hence, a high voltage can be applied between the source and the drain of the OS transistor. Therefore, when an OS transistor is used as the driving transistor in the pixel circuit, the amount of current flowing through the light-emitting device can be increased, so that the luminance of the light-emitting device can be increased.

Regarding saturation characteristics of a current flowing when transistors operate in a saturation region, even in the case where the source-drain voltage of an OS transistor increases gradually, a more stable current (saturation current) can be fed through the OS transistor than through a Si transistor. Thus, by using an OS transistor as the driving transistor, a stable current can be fed through light-emitting devices even when the current-voltage characteristics of the light-emitting devices vary, for example. In other words, when the OS transistor operates in the saturation region, the source-drain current hardly changes with an increase in the source-drain voltage; hence, the luminance of the light-emitting device can be stable.

As described above, by using OS transistors as the driving transistors included in the pixel circuits, it is possible to suppress black-level degradation, increase the luminance, increase the number of gray levels, and suppress variations in light-emitting devices, for example.

The semiconductor layer preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

It is particularly preferable that an oxide containing indium (In), gallium (Ga), and zinc (Zn) (also referred to as IGZO) be used for the semiconductor layer. It is preferable to use an oxide containing indium, tin, and zinc. It is preferable to use an oxide containing indium, gallium, tin, and zinc. It is preferable to use an oxide containing indium (In), aluminum (Al), and zinc (Zn) (also referred to as IAZO). It is preferable to use an oxide containing indium (In), aluminum (Al), gallium (Ga), and zinc (Zn) (also referred to as IAGZO). Alternatively, it is preferable to use an oxide containing indium (also referred to as IO).

When the semiconductor layer is an In-M-Zn oxide, the atomic proportion of In is preferably higher than or equal to the atomic proportion of M in the In-M-Zn oxide. Examples of the atomic ratio of the metal elements in such an In-M-Zn oxide are In:M:Zn=1:1:1, 1:1:1.2, 2:1:3, 3:1:2, 4:2:3, 4:2:4.1, 5:1:3, 5:1:6, 5:1:7, 5:1:8, 6:1:6, and 5:2:5 and a composition in the neighborhood of any of the above atomic ratios. Note that the neighborhood of the atomic ratio includes ±30% of an intended atomic ratio.

When the atomic ratio is described as In:Ga:Zn=4:2:3 or a composition in the neighborhood thereof, the case is included where the atomic proportion of Ga is greater than or equal to 1 and less than or equal to 3 and the atomic proportion of Zn is greater than or equal to 2 and less than or equal to 4 with the atomic proportion of In being 4. In addition, when the atomic ratio is described as In:Ga:Zn=5:1:6 or a composition in the neighborhood thereof, the case is included where the atomic proportion of Ga is greater than 0.5 and less than or equal to 2 and the atomic proportion of Zn is greater than or equal to 5 and less than or equal to 7 with the atomic proportion of In being 5. Furthermore, when the atomic ratio is described as In:Ga:Zn=1:1:1 or a composition in the neighborhood thereof, the case is included where the atomic proportion of Ga is greater than 0.5 and less than or equal to 2 and the atomic proportion of Zn is greater than 0.5 and less than or equal to 2 with the atomic proportion of In being 1.

The transistors included in the circuit 356 and the transistors included in the pixel portion 177 may have the same structure or different structures. One structure or two or more kinds of structures may be employed for a plurality of transistors included in the circuit 356. Similarly, one structure or two or more kinds of structures may be employed for a plurality of transistors included in the pixel portion 177.

All transistors included in the pixel portion 177 may be OS transistors, or all transistors included in the pixel portion 177 may be Si transistors. Alternatively, some of the transistors included in the pixel portion 177 may be OS transistors and the others may be Si transistors.

For example, when both an LTPS transistor and an OS transistor are used in the pixel portion 177, the display device can have low power consumption and high driving capability. Note that a structure in which an LTPS transistor and an OS transistor are used in combination is referred to as LTPO in some cases. For example, it is preferable that an OS transistor be used as a transistor functioning as a switch for controlling electrical continuity between wirings and an LTPS transistor be used as a transistor for controlling a current.

For example, one transistor included in the pixel portion 177 functions as a transistor for controlling a current flowing through the light-emitting device and can be referred to as a driving transistor. One of a source and a drain of the driving transistor is electrically connected to the pixel electrode of the light-emitting device. An LTPS transistor is preferably used as the driving transistor. In that case, the amount of current flowing through the light-emitting device can be increased in the pixel circuit.

Another transistor included in the pixel portion 177 functions as a switch for controlling selection or non-selection of a pixel and can also be referred to as a selection transistor. A gate of the selection transistor is electrically connected to a gate line, and one of a source and a drain thereof is electrically connected to a source line (signal line). An OS transistor is preferably used as the selection transistor. In that case, the gray level of the pixel can be maintained even with an extremely low frame frequency (e.g., lower than or equal to 1 fps); thus, power consumption can be reduced by stopping the driver in displaying a still image.

As described above, the display device of one embodiment of the present invention can have all of a high aperture ratio, high resolution, high display quality, and low power consumption.

Note that the display device of one embodiment of the present invention has a structure including the OS transistor and the light-emitting device having a metal maskless (MML) structure. This structure can significantly reduce a leakage current that would flow through a transistor and a leakage current that would flow between adjacent light-emitting devices (sometimes referred to as a horizontal leakage current or a lateral leakage current). Displaying images on the display device having this structure can bring one or more of image crispness, image sharpness, high color saturation, and a high contrast ratio to the viewer. When a leakage current that would flow through the transistor and a lateral leakage current that would flow between the light-emitting devices are extremely low, leakage of light at the time of black display (black-level degradation) or the like can be minimized.

In particular, in the case where a light-emitting device having an MML structure employs a side-by-side (SBS) structure, which is the above-described structure for separately forming or coloring light-emitting layers, a layer provided between light-emitting devices (for example, also referred to as an organic layer or a common layer which is shared by the light-emitting devices) is disconnected; accordingly, side leakage can be prevented or be made extremely low.

A connection portion 204 is provided in a region of the substrate 351 not overlapping with the substrate 352. In the connection portion 204, the wiring 355 is electrically connected to the FPC 353 through a conductive layer 166 and a connection layer 242. As an example, the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### [Display Device 600C]

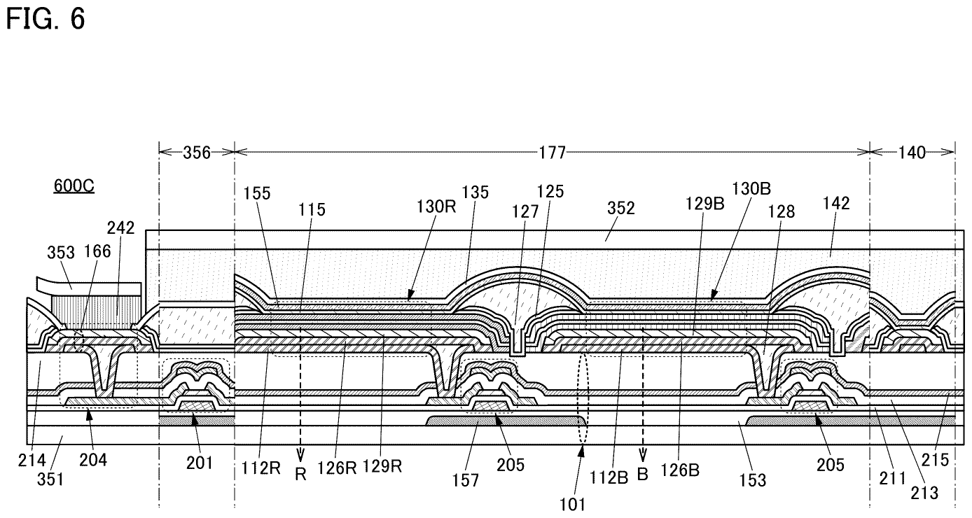

The display device 600C illustrated in FIG. 6 differs from the display device 600B illustrated in FIG. 5 mainly in having a bottom-emission structure.

Light from the light-emitting device is emitted toward the substrate 351. For the substrate 351, a material with a high visible-light-transmitting property is preferably used. By contrast, there is no limitation on the light-transmitting property of a material used for the substrate 352.

The light-blocking layer 157 is preferably formed between the substrate 351 and the transistor 201 and between the substrate 351 and the transistor 205. FIG. 6 illustrates an example in which the light-blocking layer 157 is provided over the substrate 351, an insulating layer 153 is provided over the light-blocking layer 157, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material with a high visible-light-transmitting property is used for each of the conductive layers 112R, 112B, 126R, 126B, 129R, and 129B. A material that reflects visible light is preferably used for the common layer 155.

Although not illustrated in FIG. 6, the light-emitting device 130G is also provided.

Although FIG. 6 and the like illustrate an example where the top surface of the layer 128 includes a flat portion, the shape of the layer 128 is not particularly limited.

This embodiment can be combined as appropriate with the other embodiments or the examples. In this specification, in the case where a plurality of structure examples are shown in one embodiment, the structure examples can be combined as appropriate.

### Embodiment 4

In this embodiment, electronic appliances of embodiments of the present invention will be described.

Electronic appliances of this embodiment include the display device of one embodiment of the present invention in their display portions. The display device of one embodiment of the present invention is highly reliable and can be easily increased in resolution and definition. Thus, the display device of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the display device of one embodiment of the present invention can have high resolution, and thus can be favorably used for an electronic appliance having a relatively small display portion. Examples of such an electronic appliance include watch-type and bracelet-type information terminals (wearable devices) and wearable devices capable of being worn on a head, such as a VR device like a head-mounted display, a glasses-type AR device, and an MR device.

The definition of the display device of one embodiment of the present invention is preferably as high as HD (number of pixels: 1280×720), FHD (number of pixels: 1920×1080), WQHD (number of pixels: 2560×1440), WQXGA (number of pixels: 2560×1600), 4K (number of pixels: 3840×2160), or 8K (number of pixels: 7680×4320). In particular, definition of 4K, 8K, or higher is preferable. The pixel density (resolution) of the display device of one embodiment of the present invention is preferably higher than or equal to 100 ppi, further preferably higher than or equal to 300 ppi, still further preferably higher than or equal to 500 ppi, yet still further preferably higher than or equal to 1000 ppi, yet still further preferably higher than or equal to 2000 ppi, yet still further preferably higher than or equal to 3000 ppi, yet still further preferably higher than or equal to 5000 ppi, yet still further preferably higher than or equal to 7000 ppi. With such a display device having high definition and/or high resolution, the electronic appliance can provide higher realistic sensation, sense of depth, and the like. There is no particular limitation on the screen ratio (aspect ratio) of the display device of one embodiment of the present invention. For example, the display device is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

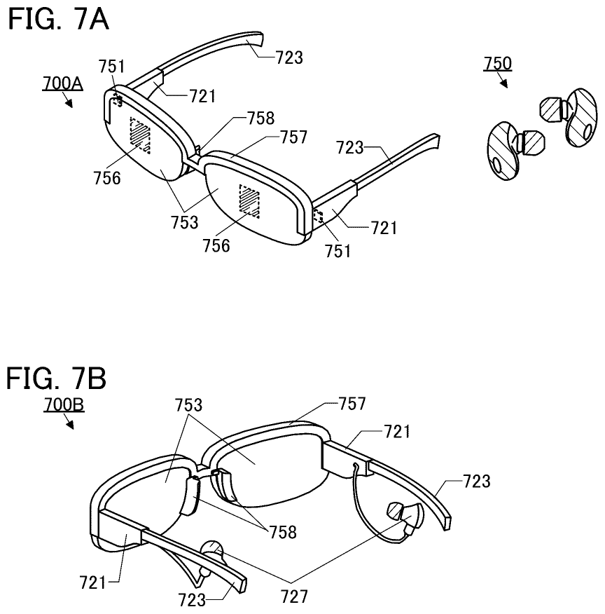

Examples of a wearable device capable of being worn on a head will be described with reference to FIGS. 7A and 7B. These wearable devices have at least one of a function of displaying AR contents, a function of displaying VR contents, a function of displaying SR contents, and a function of displaying MR contents. The electronic appliance having a function of displaying contents of at least one of AR, VR, SR, MR, and the like enables the user to feel a higher level of immersion.

An electronic appliance 700A illustrated in FIG. 7A and an electronic appliance 700B illustrated in FIG. 7B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The display device of one embodiment of the present invention can be used for the display panels 751. Thus, a highly reliable electronic appliance is obtained.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliances 700A and 700B are electronic appliances capable of performing AR display.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic appliance 700A in FIG. 7A has a function of transmitting information to the earphones 750 with the wireless communication function.

The electronic appliance may include an earphone portion. The electronic appliance 700B in FIG. 7B includes earphone portions 727. For example, the earphone portion 727 can be connected to the control portion by wire. Part of a wiring that connects the earphone portion 727 and the control portion may be positioned inside the housing 721 or the wearing portion 723.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

Note that the electronic appliance of one embodiment of the present invention can be suitably applied to a goggles-type structure without being limited to the glasses-type structure like the electronic appliances 700A and 700B.

The electronic appliance of one embodiment of the present invention can transmit information to earphones by wire or wirelessly.

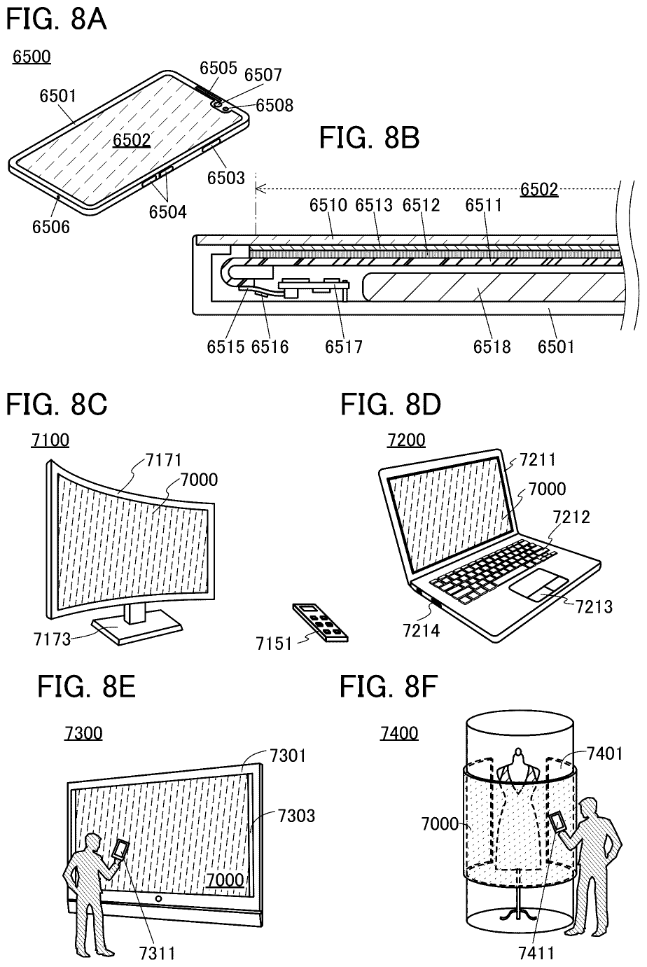

An electronic appliance 6500 illustrated in FIG. 8A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The display device of one embodiment of the present invention can be used in the display portion 6502. Thus, a highly reliable electronic appliance is obtained.

FIG. 8B is a schematic cross-sectional view including an end portion of the housing 6501 closer to the microphone 6506.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with a bonding layer (not shown).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The display device of one embodiment of the present invention can be used in the display panel 6511. Thus, an extremely lightweight electronic appliance can be achieved. Since the display panel 6511 is extremely thin, the battery 6518 with high capacity can be mounted without an increase in the thickness of the electronic appliance. Moreover, part of the display panel 6511 is folded back so that a connection portion with the FPC 6515 is provided on the back side of the pixel portion, whereby an electronic appliance with a narrow bezel can be achieved.

FIG. 8C illustrates an example of a television device. In a television device 7100, a display portion 7000 is incorporated in a housing 7171. Here, the housing 7171 is supported by a stand 7173.

The display device of one embodiment of the present invention can be used in the display portion 7000. Thus, a highly reliable electronic appliance is obtained.

Operation of the television device 7100 illustrated in FIG. 8C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel of the remote control 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 8D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The display device of one embodiment of the present invention can be used in the display portion 7000. Thus, a highly reliable electronic appliance is obtained.

FIGS. 8E and 8F illustrate examples of digital signage that can be used for store windows, showcases, and the like.

Digital signage 7300 illustrated in FIG. 8E includes a housing 7301, the display portion 7000, a speaker 7303, and the like. The digital signage 7300 can also include an LED lamp, an operation key (including a power switch or an operation switch), a connection terminal, a variety of sensors, a microphone, and the like.

FIG. 8F illustrates digital signage 7400 attached to a cylindrical pillar 7401. The digital signage 7400 includes the display portion 7000 provided along a curved surface of the pillar 7401.

In FIGS. 8E and 8F, the display device of one embodiment of the present invention can be used in the display portion 7000. Thus, a highly reliable electronic appliance is obtained.

A larger area of the display portion 7000 can increase the amount of information that can be provided at a time. The larger display portion 7000 attracts more attention, so that the effectiveness of the advertisement can be increased, for example.

Specifically, in the case where the display device of one embodiment of the present invention is used for the digital signage 7300 and the digital signage 7400 shown in FIGS. 8E and 8F that display advertisements and the like, the display device being a light-transmitting panel can increase the flexibility of representation. A light-transmitting display device can be manufactured, for example, by using a wiring and a support member each of which is formed of a conductive film that transmits visible light and adjusting the distance between pixel electrodes.

The use of the tandem light-emitting device of one embodiment of the present invention in addition to the wiring and the support member each of which is formed of the conductive film that transmits visible light can increase the luminance per pixel. That is, favorable display can be performed even when the aperture ratio of the display device is decreased; thus, the light-transmitting property of the display portion of the display device can be increased. Accordingly, such a structure is suitably used in the light-transmitting display device of one embodiment of the present invention.

As illustrated in FIGS. 8E and 8F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

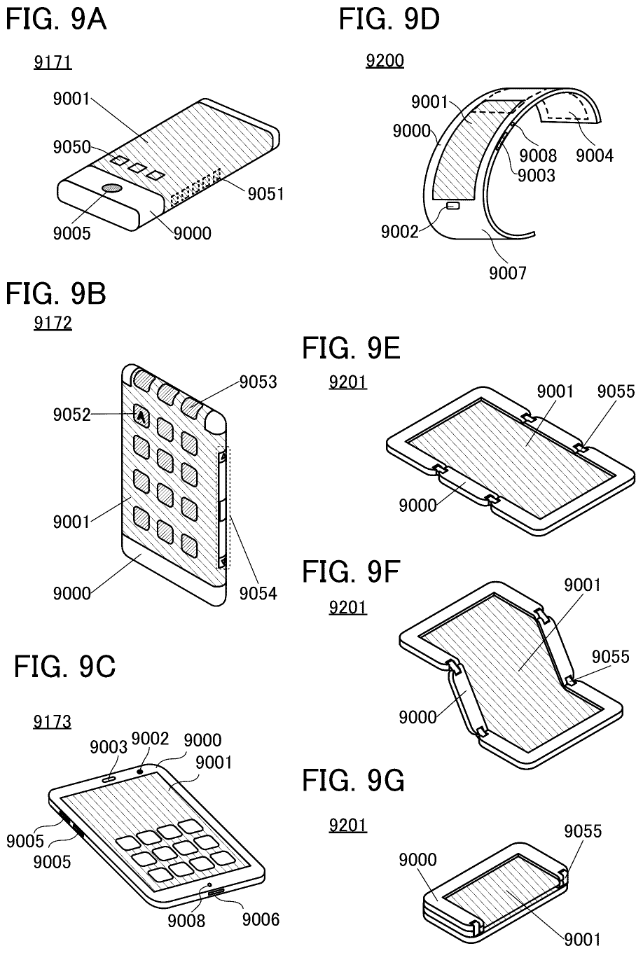

Electronic appliances illustrated in FIGS. 9A to 9G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic appliances illustrated in FIGS. 9A to 9G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with use of a variety of software (programs), a wireless communication function, and a function of reading out and processing a program or data stored in a recording medium. Note that the functions of the electronic appliances are not limited thereto, and the electronic appliances can have a variety of functions. The electronic appliances may include a plurality of display portions. The electronic appliances may be provided with a camera or the like and have a function of taking a still image or a moving image, a function of storing the taken image in a storage medium (an external storage medium or a storage medium incorporated in the camera), a function of displaying the taken image on the display portion, and the like.

The electronic appliances in FIGS. 9A to 9G will be described in detail below.

FIG. 9A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 9A illustrates an example where three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 9B is a perspective view of a portable information terminal 9172. The portable information terminal 9172 has a function of displaying information on three or more surfaces of the display portion 9001. In the example shown here, information 9052, information 9053, and information 9054 are displayed on different surfaces. For example, the user of the portable information terminal 9172 can check the information 9053 displayed such that it can be seen from above the portable information terminal 9172, with the portable information terminal 9172 put in a breast pocket of his/her clothes. Thus, the user can see the display without taking out the portable information terminal 9172 from the pocket and decide whether to answer the call, for example.

FIG. 9C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, a camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 9D is a perspective view of a watch-type portable information terminal 9200. The portable information terminal 9200 can be used as a Smartwatch (registered trademark), for example. The portable information terminal 9200 may include the operation key 9005 as a button for operation on the left side surface of the housing 9000 and the sensor 9007 on the bottom surface of the housing 9000. Although the housing 9000 having a curved bangle shape is shown as an example, a belt or the like may be used in combination with the housing 9000 to make the portable information terminal 9200 wearable. The display surface of the display portion 9001 is curved, and an image can be displayed on the curved display surface. A power storage device 9004 may have a curved shape along the housing 9000. The power storage device 9004 has flexibility and can be bent in accordance with a change in shape when the user puts on or takes off the portable information terminal 9200. Note that a charge control IC connected to the power storage device 9004 may be provided. Furthermore, for example, mutual communication between the portable information terminal 9200 and a headset capable of wireless communication can be performed, and thus hands-free calling is possible. The portable information terminal 9200 can perform mutual data transmission wirelessly with another information terminal and can be charged with wireless power feeding. Note that the connection terminal 9006 may be provided in the housing 9000 so that data transmission and charging operation may be performed by wire.

FIGS. 9E to 9G are perspective views of a foldable portable information terminal 9201. FIG. 9E is a perspective view illustrating the portable information terminal 9201 that is opened. FIG. 9G is a perspective view illustrating the portable information terminal 9201 that is folded. FIG. 9F is a perspective view illustrating the portable information terminal 9201 that is shifted from one of the states in FIGS. 9E and 9G to the other. When the portable information terminal 9201 is opened, a seamless large display region is highly browsable. The display portion 9001 of the portable information terminal 9201 is supported by three housings 9000 joined together by hinges 9055. The display portion 9001 can be folded with a radius of curvature greater than or equal to 0.1 mm and less than or equal to 150 mm, for example.

This embodiment can be combined as appropriate with the other embodiments or the examples. In this specification, in the case where a plurality of structure examples are shown in one embodiment, the structure examples can be combined as appropriate.

### Example 1

### Synthesis Example 1

Described in this synthesis example are a method for synthesizing the organic compound of one embodiment of the present invention, 2-(3-(2,6-dimethylpyridin-3-yl)-{[3′,5′-bis(trimethylsilyl)]-biphenyl}-5-yl)-4,6-diphenyl-1,3,5-triazine (abbreviation: mmTMSPh-mDMePyPTzn) (Structural Formula (100)), and the physical properties thereof.

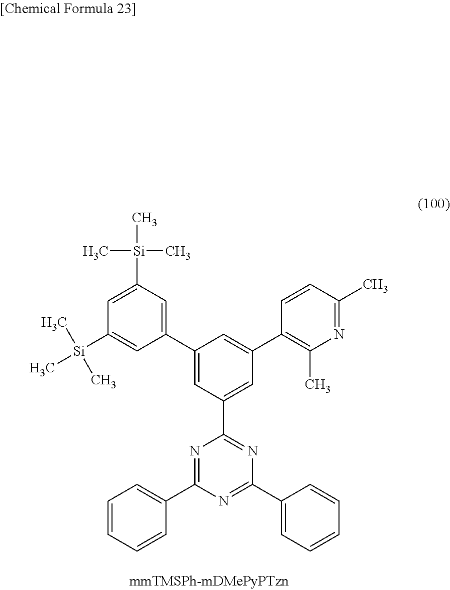

### Step 1: Synthesis of 2-[3-(2,6-dimethylpyridin-3-yl)-5-(4,6-diphenyl-1,3,5-triazine)]phenyl-4,4,5,5-tetramethyl-1,3,2-dioxaborolane

Into a three-neck flask were put 7.0 g (16 mmol) of 2-[3-chloro-5-(2,6-dimethylpyridin-3-yl)phenyl]-4,6-diphenyl-1,3,5-triazine, 5.9 g (23 mmol) of bis(pinacolato)diboron, 4.2 g (43 mmol) of potassium acetate, and 230 mL of 1,4-dioxane, and the mixture was degassed by being stirred under reduced pressure. After the degassing, 0.14 g (0.28 mmol) of 2-dicyclohexylphosphino-2′,4′,6′-triisopropylbiphenyl (abbreviation: XPhos) and 32 mg (0.14 mmol) of palladium(II) acetate were added thereto, and the mixture was stirred at 100° C. for 14 hours. After the reaction was completed, water was added to the obtained reaction mixture, and the mixture was separated into an organic layer and an aqueous layer. Toluene was added to the obtained aqueous layer, and extraction was performed. The resulting toluene layer and the organic layer were mixed, and magnesium sulfate was added thereto to adsorb moisture. This mixture was gravity-filtered, and the obtained filtrate was concentrated to give a pale yellow solid. This solid was purified by silica gel column chromatography with a developing solvent of toluene and ethyl acetate in a ratio of 10:1, which was then changed to 3:1. The obtained solution was concentrated to give 8.1 g of a target pale yellow solid in a yield of 96%. The synthesis scheme of Step 1 is shown in Formula (a-1) below.

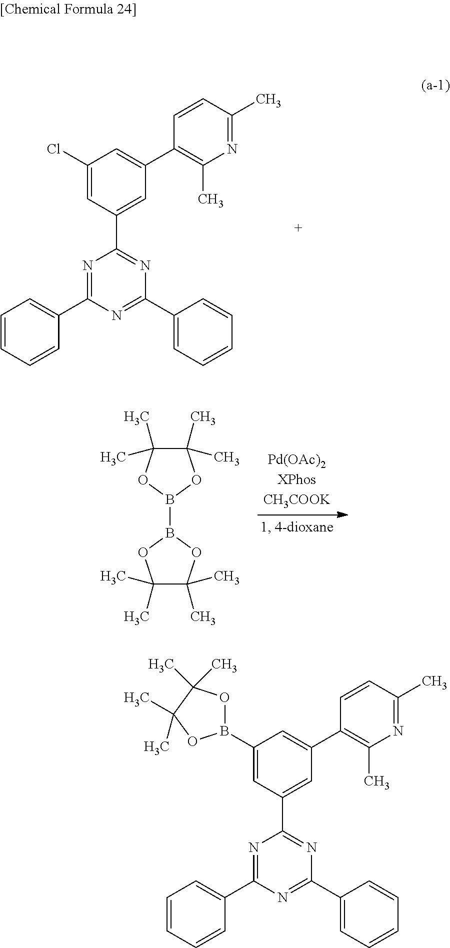

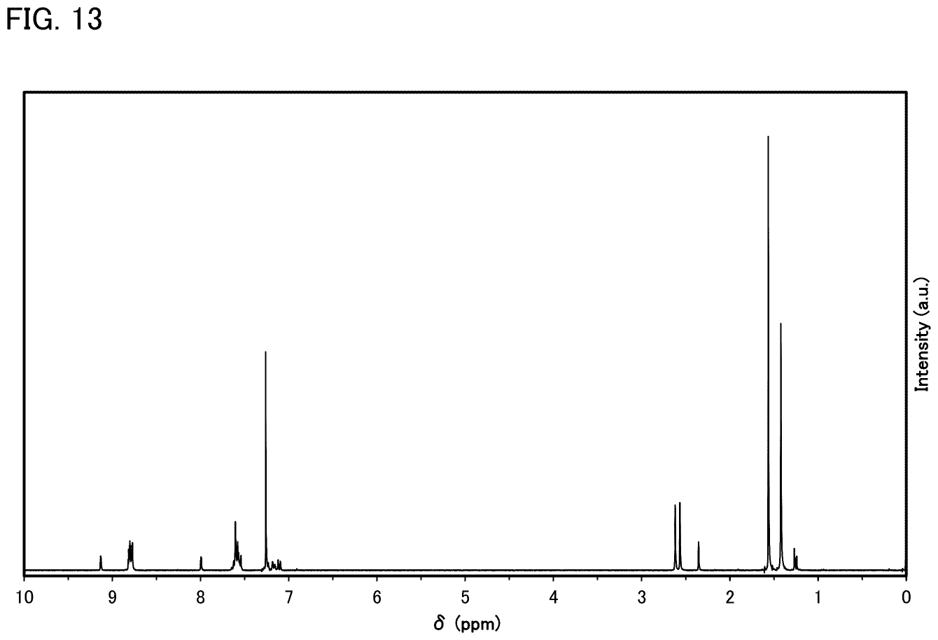

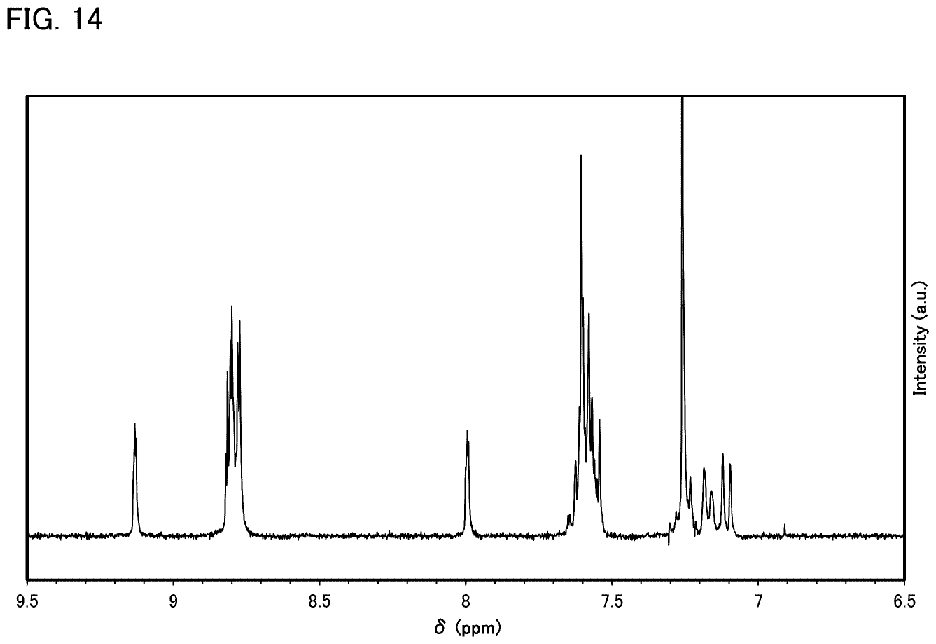

Analysis results by nuclear magnetic resonance (¹H-NMR) spectroscopy of the pale yellow solid obtained in the above Step 1 are shown below. FIG. 13 is a ¹H-NMR chart, and FIG. 14 is an enlarged chart of the range of 6.5 ppm to 9.5 ppm in FIG. 13. Note that the singlet peak at around 2.36 ppm and the multiplet peaks at around 7.17 to 7.25 ppm are derived from toluene used as the purification solvent.

¹H-NMR. δ (CDCl₃, 300 MHz): 1.42 (s, 12H), 2.57 (s, 3H), 2.62 (s, 3H), 7.11 (d, 1H, J=7.5 Hz), 7.54-7.63 (m, 7H), 7.99-8.00 (m, 1H), 8.77-8.82 (m, 5H), 9.13-9.14 (m, 1H).

### Step 2: Synthesis of mmTMSPh-mDMePyPTzn

Into a three-neck flask were put 1.3 g (2.4 mmol) of 2-[3-(2,6-dimethylpyridin-3-yl)-5-(4,6-diphenyl-1,3,5-triazine)]phenyl-4,4,5,5-tetramethyl-1,3,2-dioxaborolane obtained in Step 1, 0.66 g (2.2 mmol) of 3,5-bis(trimethylsilyl)bromobenzene, 0.60 g (4.4 mmol) of potassium carbonate, 25 mL of toluene, 5 mL of ethanol, and 3 mL of water, and the mixture was degassed by being stirred under reduced pressure. After the degassing, 90 mg (0.22 mmol) of 2-dicyclohexylphosphino-2′,6′-dimethoxybiphenyl (abbreviation: SPhos) and 9.8 mg (48 μmol) of palladium(II) acetate were added thereto, and the mixture was stirred at 80° C. for 14 hours. After the reaction was completed, the mixture was separated into an organic layer and an aqueous layer. The obtained aqueous layer was subjected to extraction with toluene, followed by mixing with the obtained organic layer, and magnesium sulfate was added thereto to adsorb moisture. This mixture was gravity-filtered, and the obtained filtrate was concentrated to give a yellow solid. This solid was purified by silica gel column chromatography with a developing solvent of toluene and ethyl acetate in a ratio of 30:1, which was then changed to 20:1, giving 1.19 g of a pale yellow solid. This solid was recrystallized with toluene and ethanol to give 0.88 g of a target white solid in a yield of 58%. By a train sublimation method, 0.87 g of the obtained solid was purified. In the purification by sublimation, the solid was heated at 235° C. under a pressure of 5.8 Pa for 18 hours. After the sublimation purification, 0.77 g of a white solid was obtained at a yield of 89%. The synthesis scheme of Step 2 is shown in (a-2) below.

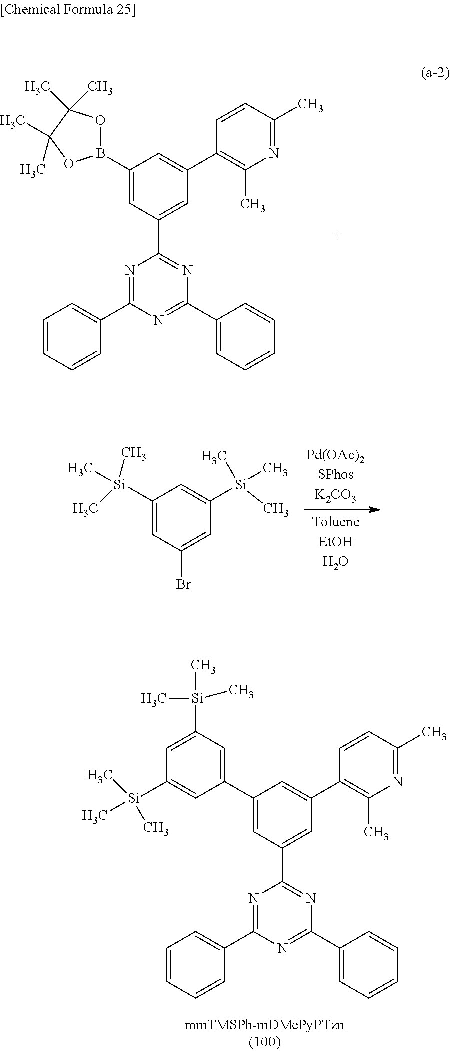

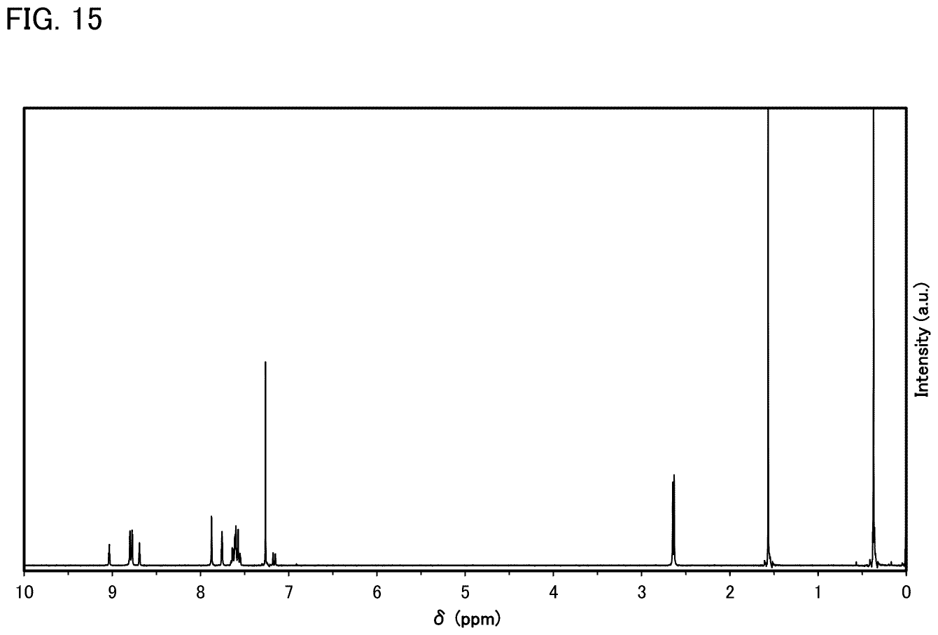

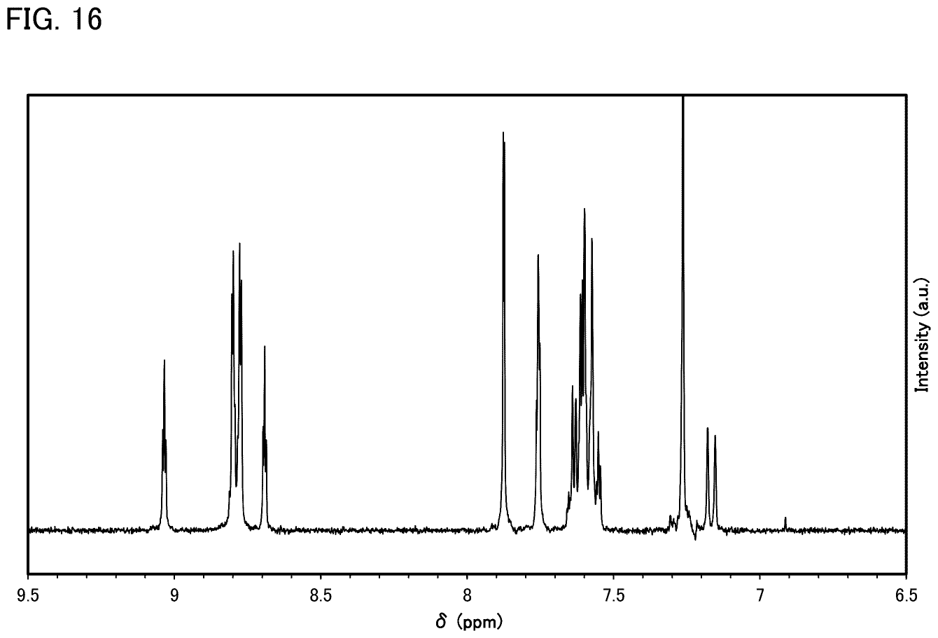

Analysis results by nuclear magnetic resonance (¹H-NMR) spectroscopy of the white solid obtained in the above Step 2 are shown below. FIG. 15 is a ¹H-NMR chart, and FIG. 16 is an enlarged chart of the range of 6.5 ppm to 9.5 ppm in FIG. 15. These results reveal that the organic compound of one embodiment of the present invention, mmTMSPh-mDMePyPTzn, was obtained in this example.

¹H-NMR. δ (CDCl₃, 300 MHz): 0.37 (s, 18H), 2.63 (s, 3H), 2.65 (s, 3H), 7.17 (d, 1H, J=8.1 Hz), 7.55-7.65 (m, 7H), 7.75-7.76 (m, 2H), 7.88 (d, 2H, J=0.9 Hz), 8.69 (t, 1H, J=1.5 Hz), 8.77-8.80 (m, 4H), 9.04 (t, 1H, J=1.7 Hz).

### <Measurement of Emission and Absorption Spectra>

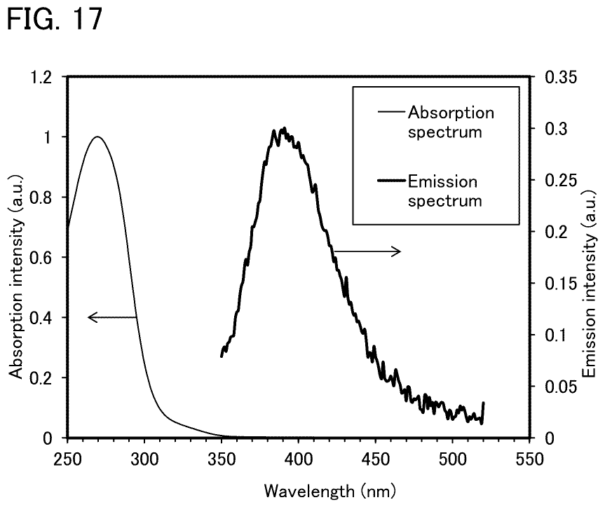

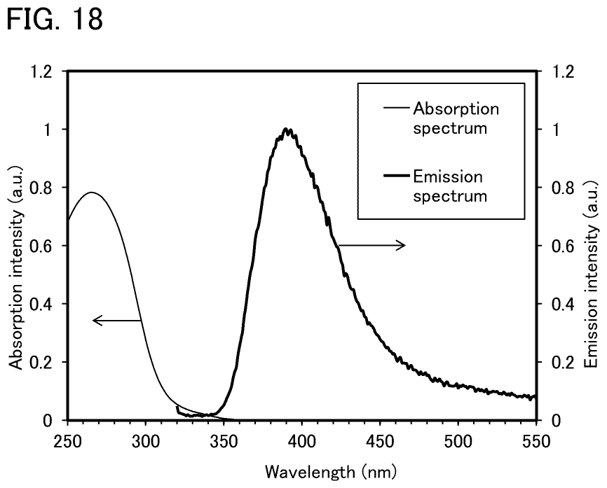

FIG. 17 shows the measurement results of an absorption spectrum and an emission spectrum of a dichloromethane solution of mmTMSPh-mDMePyPTzn. FIG. 18 shows the absorption and emission spectra of a thin film of mmTMSPh-mDMePyPTzn. The thin film was formed over a quartz substrate by a vacuum evaporation method. The absorption spectrum of the dichloromethane solution was measured with an ultraviolet-visible light spectrophotometer (V-770DS, manufactured by JASCO Corporation), and the spectrum of dichloromethane alone in a quartz cell was subtracted. The absorption spectrum of the thin film was measured with a spectrophotometer (U-4100 Spectrophotometer, manufactured by Hitachi High-Technologies Corporation). The emission spectra were measured with a fluorescence spectrophotometer (FP-8600, manufactured by JASCO Corporation).

As shown in FIG. 17, the dichloromethane solution of mmTMSPh-mDMePyPTzn has an absorption spectrum peak at a wavelength of 270 nm and an emission spectrum peak at a wavelength of 390 nm (excitation wavelength: 270 nm). As shown in FIG. 18, the thin film of mmTMSPh-mDMePyPTzn has an absorption spectrum peak at a wavelength of 265 nm and an emission spectrum peak at a wavelength of 390 nm (excitation wavelength: 310 nm). It was found from FIG. 17 and FIG. 18 that mmTMSPh-mDMePyPTzn exhibits no absorption within the visible range (at wavelengths longer than 450 nm).

### <Measurement of Refractive Index>

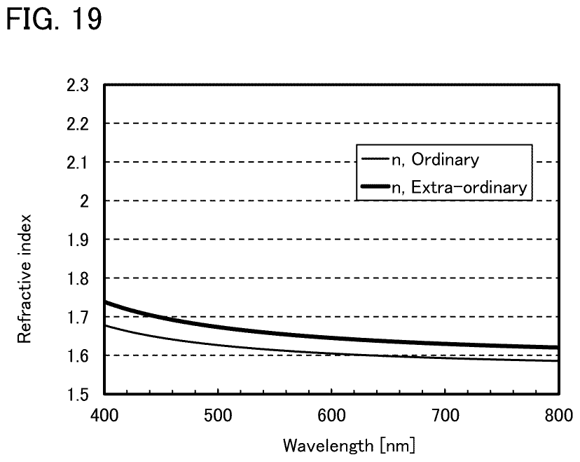

FIG. 19 shows the results of measuring the refractive index of the mmTMSPh-mDMePyPTzn film with a spectroscopic ellipsometer (M-2000U, produced by J. A. Woollam Japan Corp.). The film used for the measurement was formed to a thickness of approximately 50 nm with the material over a quartz substrate by a vacuum evaporation method. Note that a refractive index for an ordinary ray, n, Ordinary, and a refractive index for an extraordinary ray, n, Extra-ordinary, are shown in FIG. 19.

As shown in FIG. 19, the mmTMSPh-mDMePyPTzn film has an ordinary refractive index in the range of 1.50 to 1.75 for the entire blue emission region (at wavelengths of 455 nm and 465 nm) and also has an ordinary refractive index at a wavelength of 633 nm in the range of 1.45 to 1.70, indicating that the film has a low refractive index.

### <Measurement of GSP Slope>

Next, the GSP slope of an evaporated film of mmTMSPh-mDMePyPTzn was measured. The measurement was performed by the method described in Embodiment 1. The results are shown in Table 3. Table 3 also shows the GSP slope of an evaporated film of 2-{3-(2,6-dimethylpyridin-3-yl)-5-[(3,5-di-tert-butyl)phenyl]phenyl}-4,6-diphenyl-1,3,5-triazine (abbreviation: mmtBuPh-mDMePyPTzn) for comparison. The chemical formula of mmTMSPh-mDMePyPTzn, which is the organic compound of one embodiment of the present invention, and the chemical formula of mmtBuPh-mDMePyPTzn, which is a comparative organic compound, are shown below.

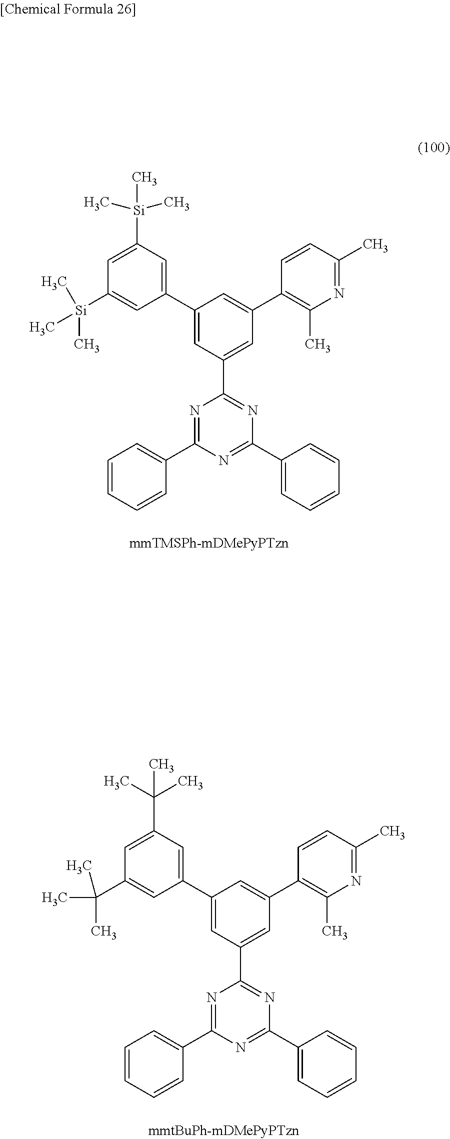

TABLE 3

| | GSP slope (mV/nm) |
|---|---|
| mmTMSPh-mDMePyPTzn | 35.0 |
| mmtBuPh-mDMePyPTzn | 44.3 |

As shown in Table 3, mmTMSPh-mDMePyPTzn, which is the organic compound of one embodiment of the present invention, has a smaller GSP slope in the form of a film than mmtBuPh-mDMePyPTzn, which is a comparative organic compound. In mmTMSPh-mDMePyPTzn, the trisubstituted silyl groups have a structure in which the quaternary carbon atoms of the two tert-butyl groups of mmtBuPh-mDMePyPTzn are each replaced with a silicon atom. It can be considered that, since a silicon atom has lower electronegativity than a carbon atom, mmTMSPh-mDMePyPTzn had a smaller permanent electric dipole moment than mmtBuPh-mDMePyPTzn and the SOP of the evaporated film of mmTMSPh-mDMePyPTzn decreased accordingly, which resulted in the small GSP slope.

### <Calculation of Permanent Electric Dipole Moment>

Next analyzed were the permanent electric dipole moments of the stable structures of the singlet ground states of mmTMSPh-mDMePyPTzn, which is the organic compound of one embodiment of the present invention, and mmtBuPh-mDMePyPTzn, which is a comparative organic compound.

A density functional theory (DFT) method was used as the calculation method. As a functional, B3LYP was used, and as a basis function, 6-311G(d,p) was used. Gaussian 16 was used as a computational program.

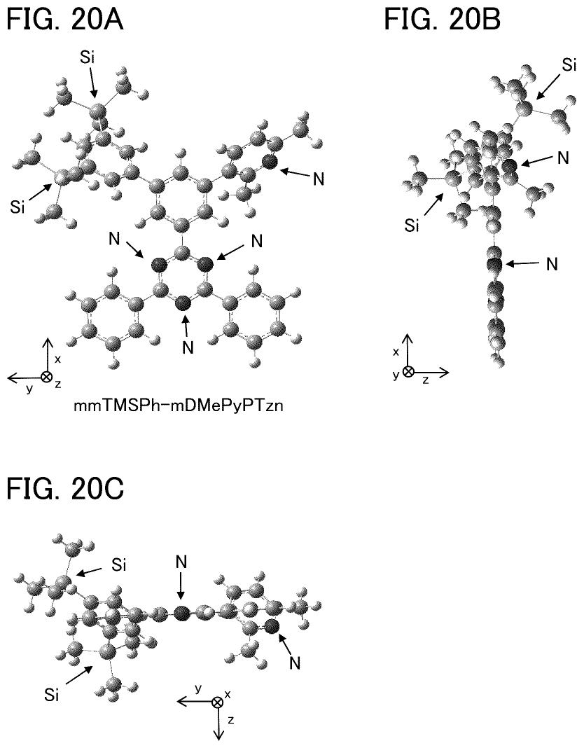

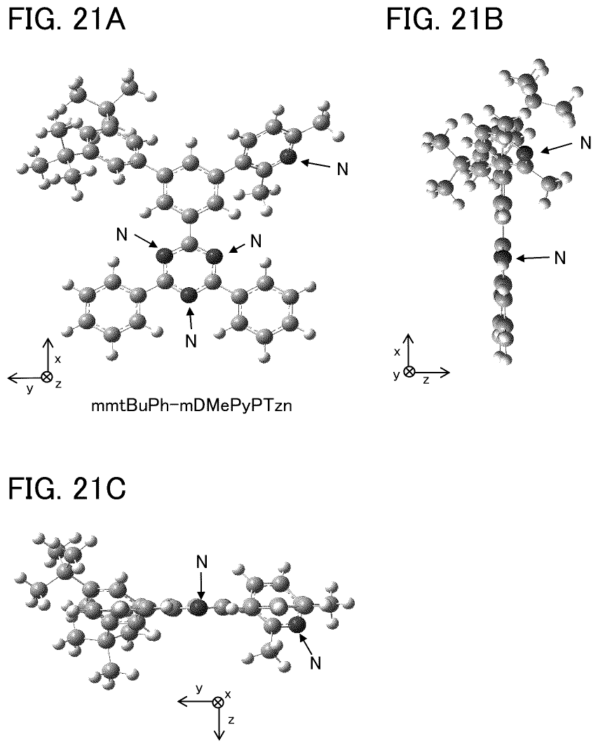

FIG. 20A illustrates the stable structure of mmTMSPh-mDMePyPTzn used for the calculation. FIG. 20B illustrates the stable structure of mmTMSPh-mDMePyPTzn in FIG. 20A seen from the y-axis direction, and FIG. 20C illustrates the stable structure seen from the x-axis direction. FIG. 21A illustrates the stable structure of mmtBuPh-mDMePyPTzn used for the calculation. FIG. 21B illustrates the stable structure of mmtBuPh-mDMePyPTzn in FIG. 21A seen from the y-axis direction, and FIG. 21C shows the stable structure seen from the x-axis direction. As can be seen from FIGS. 20A to 20C and FIGS. 21A to 21C, the stable structures of mmTMSPh-mDMePyPTzn and mmtBuPh-mDMePyPTzn used for the calculation have similar conformations. That is, the calculation for the stable structures was conducted on the assumption that their initial structures have the same conformation except for the trimethylsilyl group and the tert-butyl group. In other words, a comparison was made unaffected by conformational differences other than the difference of the trimethylsilyl group and the tert-butyl group.

Table 4 shows the magnitudes of the permanent electric dipole moments of mmTMSPh-mDMePyPTzn and mmtBuPh-mDMePyPTzn that were obtained by the calculation.

TABLE 4

| | Permanent electric |
|---|---|
| | dipole moment (Debye) |
| mmTMSPh-mDMePyPTzn | 1.66 |
| mmtBuPh-mDMePyPTzn | 1.89 |

Table 4 shows that mmTMSPh-mDMePyPTzn has a smaller permanent electric dipole moment than mmtBuPh-mDMePyPTzn. The same trend was found by comparing the permanent electric dipole moments of another mmTMSPh-mDMePyPTzn, whose conformation is similar to that in FIGS. 20A to 20C and where conformation of the stable structure of the substituents is different from conformation of the stable structure of the substituents in FIGS. 20A to 20C, with another mmtBuPh-mDMePyPTzn, whose conformation is similar to that in FIGS. 21A to 21C and where conformation of the stable structure of the substituents is different from conformation of the stable structure of the substituents in FIGS. 21A to 21C. For example, also when a comparison was made between a structure formed by rotating the dimethylpyridinyl group in the stable structure of mmTMSPh-mDMePyPTzn illustrated in FIG. 20A by 1800 around the bond between the dimethylpyridinyl group and the benzene ring to which the dimethylpyridinyl group is bonded and a structure formed by rotating the dimethylpyridinyl group in the stable structure of mmtBuPh-mDMePyPTzn illustrated in FIG. 21A by 180°, mmTMSPh-mDMePyPTzn was found to have a smaller permanent electric dipole moment than mmtBuPh-mDMePyPTzn.

According to the above results, the reason why the GSP slope of the evaporated film of mmTMSPh-mDMePyPTzn is smaller than the GSP slope of the evaporated film of mmtBuPh-mDMePyPTzn was found to be that the molecule of mmTMSPh-mDMePyPTzn has a smaller permanent electric dipole moment than that of mmtBuPh-mDMePyPTzn. This reveals that the GSP slope of the evaporated film can be made small when the trisubstituted silyl groups having a structure in which the quaternary carbon atoms included in the tert-butyl groups are each replaced with a silicon atom are introduced, instead of the tert-butyl groups, into the organic compound.

### Example 2

This example describes the fabrication of a light-emitting device G-1 and a light-emitting device G-4 each including mmTMSPh-mDMePyPTzn, which is the organic compound of one embodiment of the present invention described in Example 1, and a comparative light-emitting device G-2, a comparative light-emitting device G-3, and a comparative light-emitting device G-5 each including a comparative organic compound, and the measurement results of the device. These light-emitting devices emit green phosphorescent light. Structural formulae of organic compounds used for the light-emitting devices are shown below.

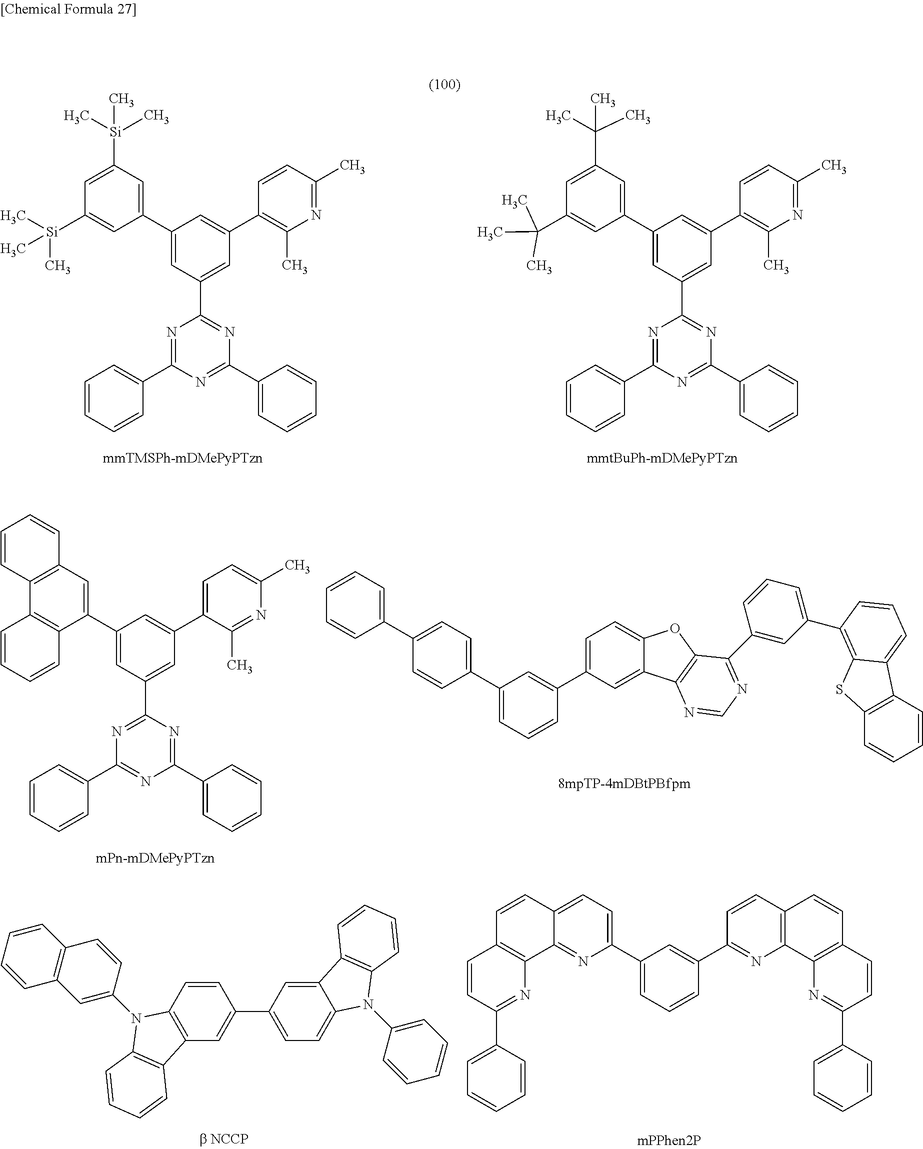

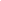

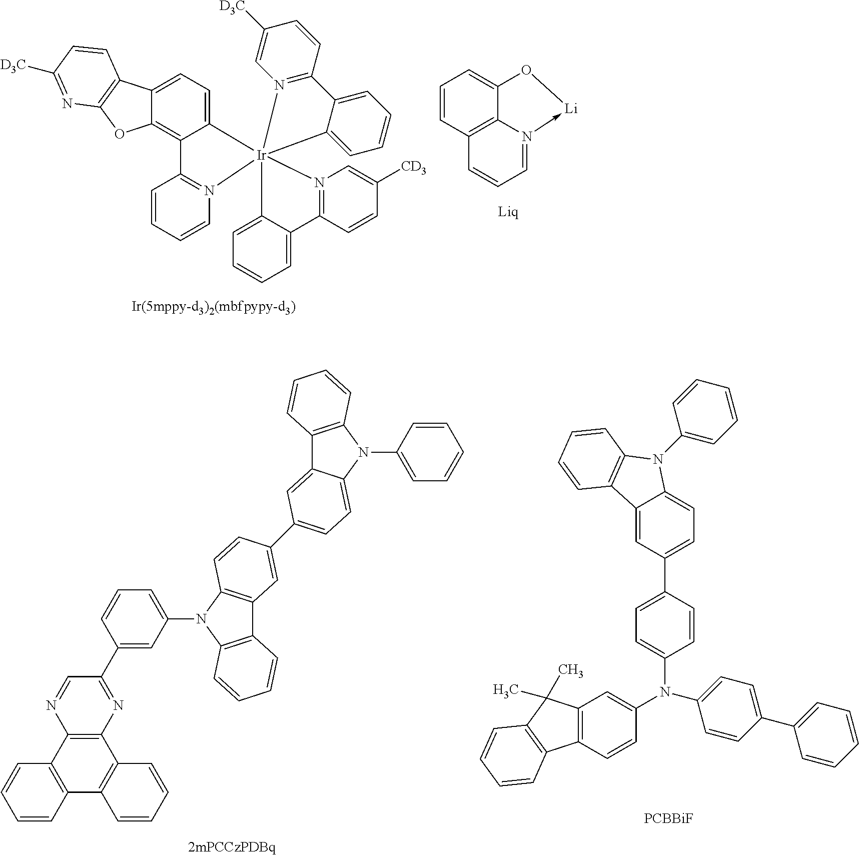

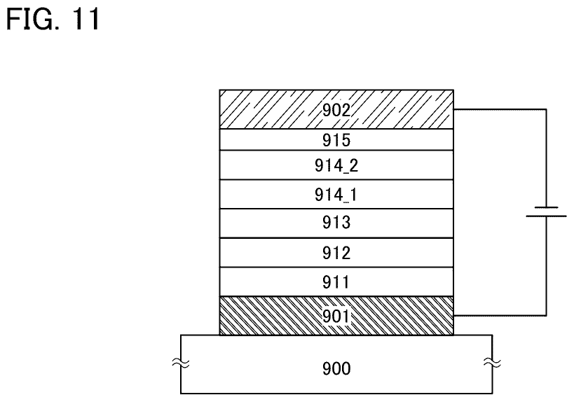

As illustrated in FIG. 11, the light-emitting devices each have a structure in which a hole-injection layer 911, a hole-transport layer 912, a light-emitting layer 913, electron-transport layers (a first electron-transport layer 914_1 and a second electron-transport layer 9142), and an electron-injection layer 915 are stacked in this order over a first electrode 901 formed over a glass substrate 900, and a second electrode 902 is formed over the second electron-injection layer 915.

### <Fabrication Method of Light-Emitting Device G-1>

Indium tin oxide containing silicon oxide (ITSO) was deposited by a sputtering method over the glass substrate 900 to a thickness of 70 nm, so that the first electrode 901 as a transparent electrode was formed. The electrode area was set to 4 mm² (2 mm×2 mm).

Next, in pretreatment for forming the light-emitting device over the substrate, the substrate surface was washed with water and baking was performed at 200° C. for 1 hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed.

Then, the substrate provided with the first electrode 901 was fixed to a substrate holder provided in the vacuum evaporation apparatus such that the surface on which the first electrode 901 was formed faced downward. Over the first electrode 901, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were co-deposited to a thickness of 10 nm by evaporation at the weight ratio of 1:0.03 (PCBBiF: OCHD-003), whereby the hole-injection layer 911 was formed.

Then, over the hole-injection layer 911, PCBBiF was deposited by evaporation to a thickness of 40 nm, whereby the hole-transport layer 912 was formed.

Then, over the second hole-transport layer 912, 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-3,3′-bi-9H-carbazole (abbreviation: βNCCP), and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) were deposited by co-evaporation to a thickness of 40 nm at the weight ratio of 0.5:0.5:0.1 (8mpTP-4mDBtPBfpm: βNCCP: Ir(5mppy-d₃)₂(mbfpypy-d₃)), whereby the light-emitting layer 913 was formed.

Next, the first electron-transport layer 914_1 was formed to a thickness of 20 nm over the light-emitting layer 913 by evaporation of 2-{3-[3-(N-phenyl-9H-carbazol-3-yl)-9H-carbazol-9-yl]phenyl}dibenzo[f,h]quinoxaline (abbreviation: 2mPCCzPDBq). Then, the second electron-transport layer 914_2 was formed to a thickness of 20 nm by co-evaporation of mmTMSPh-mDMePyPTzn, which is the organic compound of one embodiment of the present invention, and 8-quinolinolato-lithium (abbreviation: Liq) at the weight ratio of 1:1 (mmTMSPh-mDMePyPTzn: Liq).

Then, over the second electron-transport layer 9142, lithium fluoride (LiF) was deposited by evaporation to a thickness of 1 nm, whereby the electron-injection layer 915 was formed.

Next, the second electrode 902 was formed to a thickness of 100 nm over the electron-injection layer 915 by evaporation of aluminum (Al). Thus, the light-emitting device G-1 was fabricated.

### <Fabrication Method of Comparative Light-Emitting Device G-2>

The comparative light-emitting device G-2 is different from the light-emitting device G-1 in that mmTMSPh-mDMePyPTzn, which is the organic compound of one embodiment of the present invention used for the second electron-transport layer 9142, is replaced with mmtBuPh-mDMePyPTzn, which is a comparative organic compound. Other components were fabricated in a manner similar to that for the light-emitting device G-1.

### <Fabrication Method of Comparative Light-Emitting Device G-3>

The comparative light-emitting device G-3 is different from the light-emitting device G-1 in that the second electron-transport layer 914_2 was formed to a thickness of 20 nm by evaporation of 2,2′-(1,3-phenylene)bis(9-phenyl-1,10-phenanthroline) (abbreviation: mPPhen2P), which is a comparative organic compound. Other components were fabricated in a manner similar to that for the light-emitting device G-1.

### <Fabrication Method of Light-Emitting Device G-4>

The light-emitting device G-4 is different from the light-emitting device G-1 in that the first electron-transport layer 914_1 was formed to a thickness of 20 nm by evaporation of mmTMSPh-mDMePyPTzn, which is the organic compound of one embodiment of the present invention, and that the second electron-transport layer 914_2 was formed to a thickness of 20 nm by co-evaporation of 2-[3-(2,6-dimethyl-3-pyridyl)-5-(9-phenanthryl)phenyl]-4,6-diphenyl-1,3,5-triazine (abbreviation: mPn-mDMePyPTzn) and Liq at the weight ratio of 1:1 (mPn-mDMePyPTzn: Liq). Other components were fabricated in a manner similar to that for the light-emitting device G-1.

### <Fabrication Method of Comparative Light-Emitting Device G-5>

The light-emitting device G-5 is different from the light-emitting device G-4 in that mmTMSPh-mDMePyPTzn, the organic compound of one embodiment of the present invention used in the first electron-transport layer 914_1 of the light-emitting device G-4, was replaced with 2mPCCzPDBq, which is a comparative organic compound. Other components were fabricated in a manner similar to that for the light-emitting device G-4.

Table 5 lists the device structures of the light-emitting device G-1 and the comparative light-emitting devices G-2 and G-3. Table 6 lists the device structures of the light-emitting device G-4 and the comparative light-emitting device G1-5.

TABLE 5

| | | | | Light-emitting | Comparative light- | Comparative light- |
|---|---|---|---|---|---|---|
| | | Thickness | | device G-1 | emitting device G-2 | emitting device G-3 |
| Second electrode | | 100 | nm | Al | | |
| Electron-injection layer | | 1 | nm | LiF | | |
| Electron- | 2 | 20 | nm | mmTMSPh-mDMePyPTzn:Liq | mmtBuPh-mDMePyPTzn:Liq | mPPhen2P |

| | | |
|---|---|---|
| transport | (1:1) | (1:1) |

| | | | | |
|---|---|---|---|---|
| layer | 1 | 20 | nm | 2mPCCzPDBq |

| | | | |
|---|---|---|---|
| Light-emitting layer | 40 | nm | 8mpTP-4mDBtPBfpm:βNCCP:Ir(5mppy-d₃)₂(mbfpypy-d₃) (0.5:0.5:0.1) |
| Hole-transport layer | 40 | nm | PCBBiF |
| Hole-injection layer | 10 | nm | PCBBiF:OCHD-003 (1:0.03) |
| First electrode | 70 | nm | ITSO |

TABLE 6

| | | | | Light-emitting | Comparative light- |
|---|---|---|---|---|---|
| | | Thickness | | device G-4 | emitting device G-5 |
| Second electrode | | 100 | nm | Al | |
| Electron-injection layer | | 1 | nm | LiF | |
| Electron- | 2 | 20 | nm | mPn-mDMePyPTzn:Liq (1:1) | |
| transport layer | 1 | 20 | nm | mmTMSPh-mDMePyPTzn | 2mPCCzPDBq |

| | | | |
|---|---|---|---|
| Light-emitting layer | 40 | nm | 8mpTP-4mDBtPBfpm:βNCCP:Ir(5mppy-d₃)₂(mbfpypy-d₃) (0.5:0.5:0.1) |
| Hole-transport layer | 40 | nm | PCBBiF |
| Hole-injection layer | 10 | nm | PCBBiF:OCHD-003 (1:0.03) |
| First electrode | 70 | nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for one hour were performed at the time of sealing). Then, the characteristics of the light-emitting devices were measured.

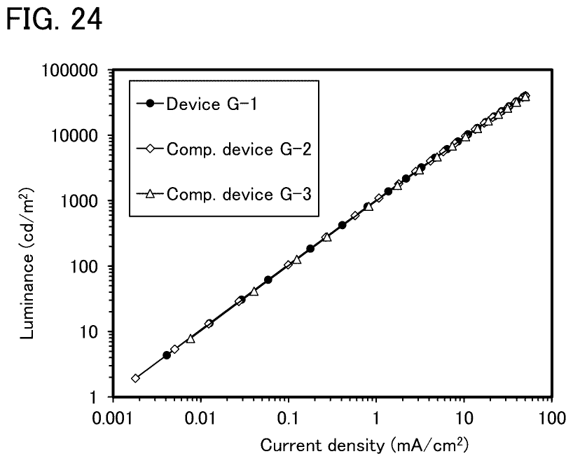

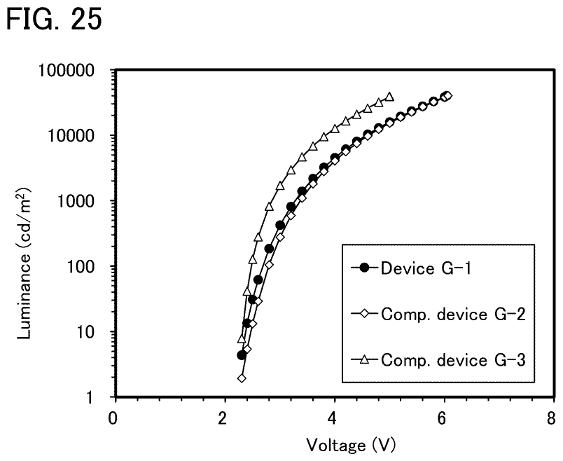

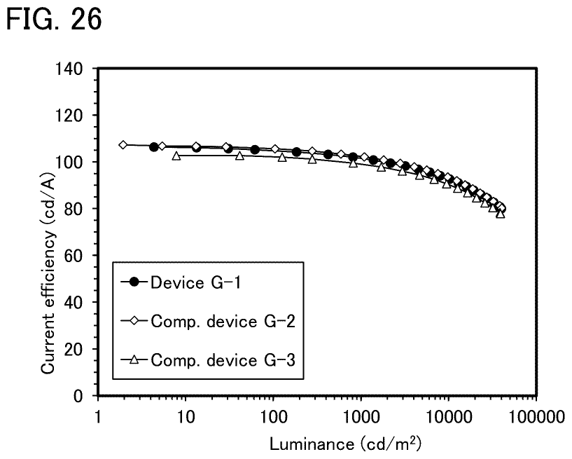

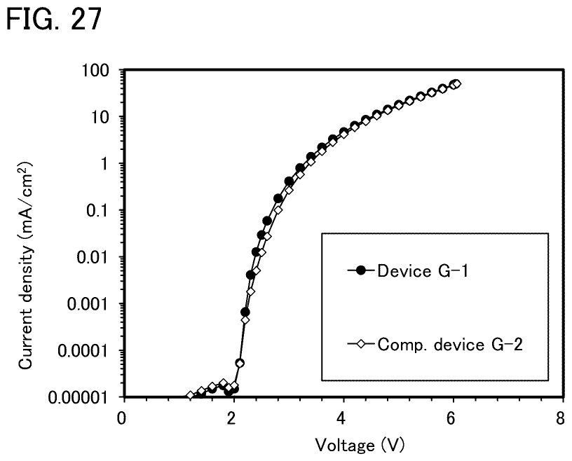

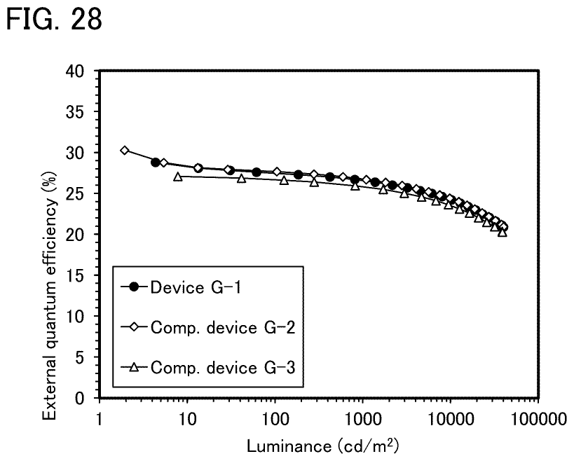

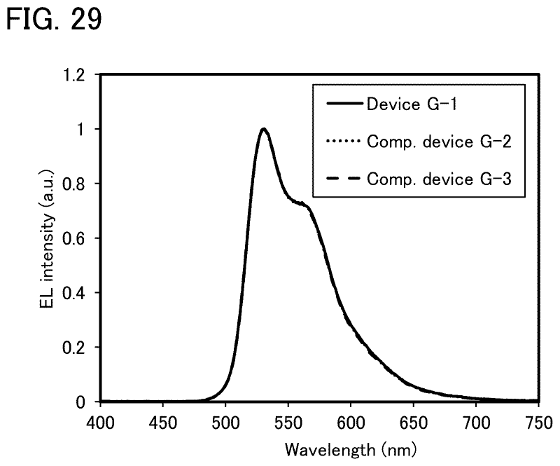

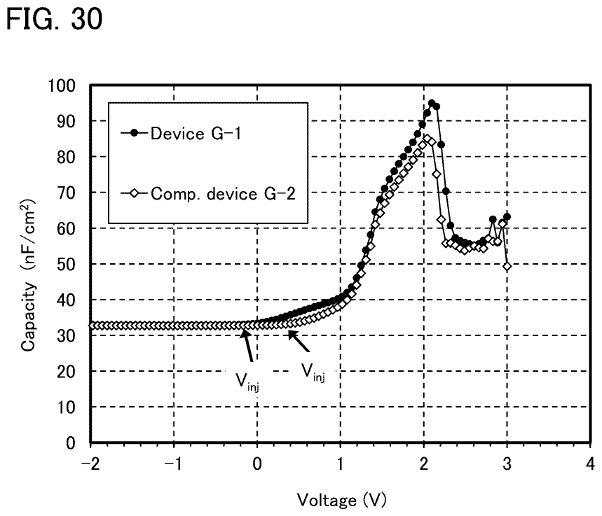

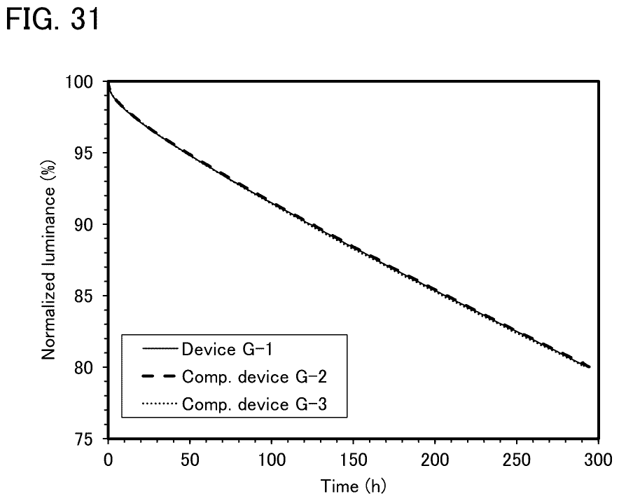

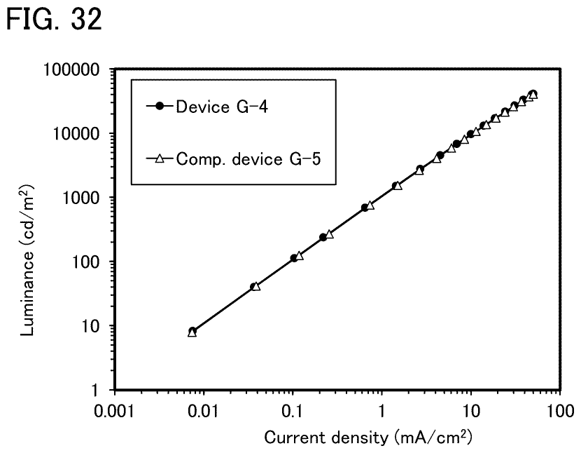

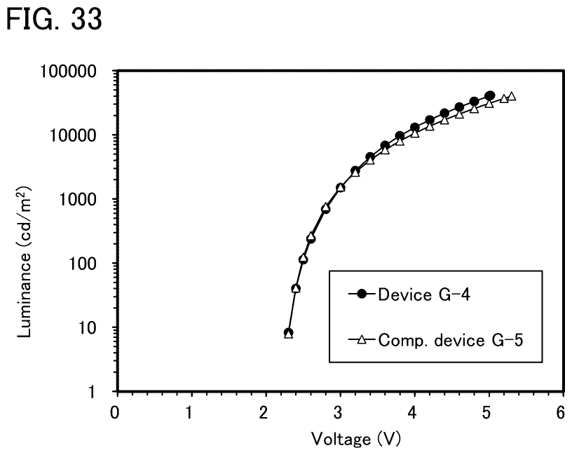

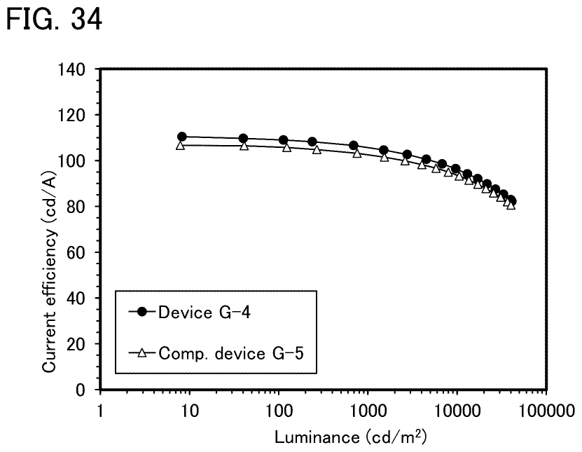

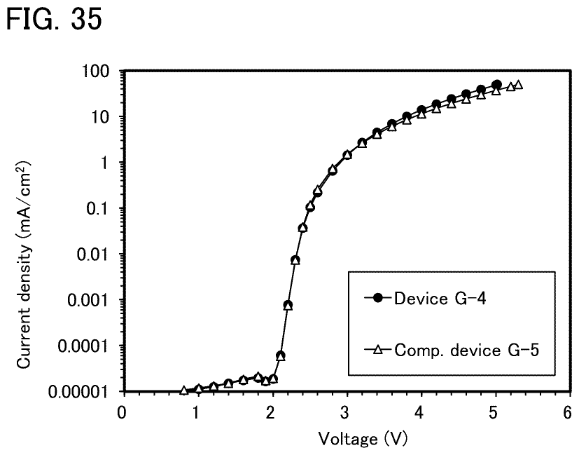

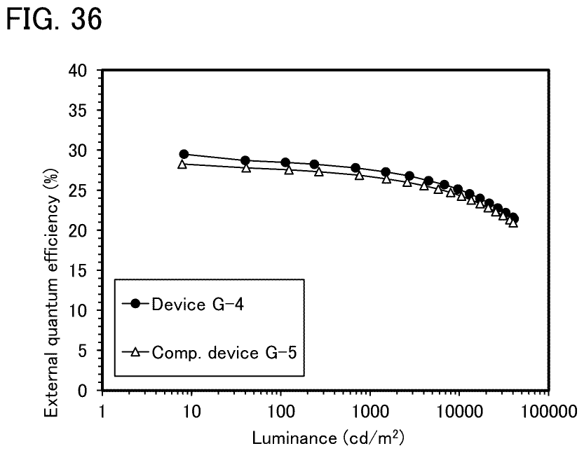

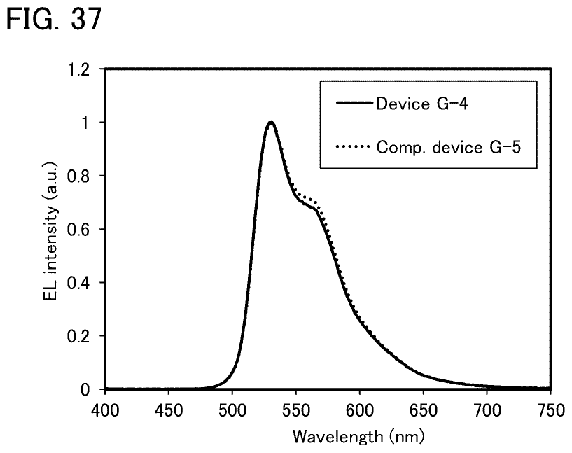

FIG. 24 shows luminance-current density characteristics of the light-emitting device G-1 and the comparative light-emitting devices G-2 and G-3. FIG. 25 shows luminance-voltage characteristics thereof. FIG. 26 shows current efficiency-luminance characteristics thereof. FIG. 28 shows external quantum efficiency-luminance characteristics thereof. FIG. 29 shows electroluminescence spectra thereof. FIG. 27 shows current density-voltage characteristics of the light-emitting device G-1 and the comparative light-emitting device G-2. FIG. 30 shows capacity-voltage characteristics thereof. FIG. 31 shows luminance changes over driving time when the light-emitting device G-1 and the comparative light-emitting devices G-2 and G-3 are driven at a constant current of 2 mA (50 mA/cm²). FIG. 32 shows luminance-current density characteristics of the light-emitting device G-4 and the comparative light-emitting device G-5. FIG. 33 shows luminance-voltage characteristics thereof. FIG. 34 shows current efficiency-luminance characteristics thereof. FIG. 35 shows current density-voltage characteristics thereof. FIG. 36 shows external quantum efficiency-luminance characteristics thereof. FIG. 37 shows electroluminescence spectra thereof. In the legends in FIG. 24 to FIG. 37, the light-emitting device G-1, the comparative light-emitting device G-2, the comparative light-emitting device G-3, the light-emitting device G-4, and the comparative light-emitting device G-5 are denoted by Device G-1, Comp. device G-2, Comp. device G-3, Device G-4, and Comp. device G-5, respectively.

Table 7 shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 7

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Light-emitting | 3.20 | 0.0318 | 0.794 | 0.369 | 0.609 | 810 | 102 | 26.7 |
| device G-1 | | | | | | | | |
| Comparative | 3.40 | 0.0428 | 1.07 | 0.369 | 0.609 | 1092 | 102 | 26.7 |
| light-emitting | | | | | | | | |
| device G-2 | | | | | | | | |
| Comparative | 2.80 | 0.0329 | 0.822 | 0.367 | 0.611 | 818 | 100 | 25.9 |
| light-emitting | | | | | | | | |
| device G-3 | | | | | | | | |
| Light-emitting | 2.80 | 0.0259 | 0.647 | 0.362 | 0.614 | 690 | 107 | 27.8 |
| device G-4 | | | | | | | | |
| Comparative | 2.80 | 0.0294 | 0.736 | 0.366 | 0.612 | 759 | 103 | 26.9 |
| light-emitting | | | | | | | | |
| device G-5 | | | | | | | | |

From FIG. 24 to FIG. 37 and Table 7, the light-emitting devices G-1 and G-4 were found to be light-emitting devices with favorable characteristics that emit green light derived from Ir(5mppy-d₃)₂(mbfpypy-d₃).

FIG. 25 and FIG. 27 show that the driving voltage of the light-emitting device G-1 is lower than that of the comparative light-emitting device G-2 at low luminance or low current density. FIG. 26 and FIG. 28 show that the light-emitting device G-1 has higher emission efficiency than the comparative light-emitting device G-3.

FIG. 30 shows that Vᵢₙⱼ of the light-emitting device G-1 is lower than that of the comparative light-emitting device G-2. Note that Vᵢₙⱼ represents a voltage required for injection of electrons from the electron-injection layer 915 or the second electrode 902 into the second electron-transport layer 914_2. This indicates that the light-emitting device G-1 enables electrons to be injected into the second electron-transport layer 914_2 at a lower voltage than the comparative light-emitting device G-2.

FIG. 31 shows that the light-emitting device G-1 has a luminance change over driving time, which is equivalent to those of the comparative light-emitting device G-2 and the comparative light-emitting device G-3, and has a long lifetime.

FIG. 33 and FIG. 35 show that the light-emitting device G-4 has a lower driving voltage than the comparative light-emitting device G-5. FIG. 34 and FIG. 36 show that the light-emitting device G-4 has higher emission efficiency than the comparative light-emitting device G-5.

Here, Table 8 shows the ordinary refractive indices (nₒ) and GSP slopes of the evaporated films of major organic compounds used in the light-emitting devices. The ordinary refractive indices at a wavelength of 633 nm are shown as the ordinary refractive indices in Table 8. A spectroscopic ellipsometer (M-2000U, manufactured by J.A. Woollam Japan) was used to measure the ordinary refractive indices, and the material for each layer was deposited to a thickness of 50 nm over a quartz substrate by a vacuum evaporation method to form films used as measurement samples. The GSP slopes of the films of the organic compounds in Table 8 were measured by the method described in Embodiment 1.

TABLE 8

| | nₒ (@633 nm) | GSP slope (mV/nm) |
|---|---|---|
| mPPhen2P | 1.80 | 1.5 |
| 2mPCCzPDBq | 1.88 | 12.4 |
| mmtBuPh-mDMePyPTzn | 1.62 | 44.3 |
| mmTMSPh-mDMePyPTzn | 1.60 | 35.0 |

As shown in Table 8, the film of mmTMSPh-mDMePyPTzn, which is the organic compound of one embodiment of the present invention, has a lower refractive index than the films of mPPhen2P and 2mPCCzPDBq and a smaller GSP slope than the film of mmtBuPh-mDMePyPTzn.

As described above, it was found that the light-emitting device G-1 has a lower Vᵢₙⱼ than the comparative light-emitting device G-2 and is capable of injecting electrons into the second electron-transport layer 914_2 at a lower voltage than the comparative light-emitting device G-2. It was also found that the driving voltage of the light-emitting device G-1 is lower than that of the comparative light-emitting device G-2 at low luminance or low current density. These results can be explained by the fact that in the form of a film, mmTMSPh-mDMePyPTzn, which was used in the light-emitting device G-1 as shown in Table 6, has a smaller GSP slope than mmtBuPh-mDMePyPTzn, which was used in the comparative light-emitting device G-2 as shown in Table 6. In other words, the light-emitting device G-1 achieved electron injection into the second electron-transport layer 914_2 at a lower voltage owing to the use of mmTMSPh-mDMePyPTz, which has a smaller GSP slope in the form of a film, in the second electron-transport layer 9142. Furthermore, the achievement of electron injection into the second electron-transport layer 914_2 at a lower voltage enabled a reduction in the driving voltage of the light-emitting device.

The above results reveal that the light-emitting device using the organic compound of one embodiment of the present invention has a low driving voltage and high emission efficiency. This is because, in the light-emitting device, the organic compound of one embodiment of the present invention in the electron-transport layer enables higher efficiency of light extraction from the light-emitting layer and effective voltage application to the light-emitting layer since the organic compound of one embodiment of the present invention has a low refractive index and a small GSP slope in the form of a film.

Since the organic compound of one embodiment of the present invention has a small GSP slope in the form of a film and the light-emitting device can have low Vᵢₙⱼ for injection into the second electron-transport layer, a wider range of materials can be used for the first electron-transport layer 914_1 and the light-emitting layer 913 in the light-emitting device using the organic compound of one embodiment of the present invention. This indicates that the organic compound of one embodiment of the present invention can be suitably used for light-emitting devices having a variety of structures. Furthermore, the organic compound of one embodiment of the present invention is found to be suitable for use in a carrier-transport layer in a display device where the carrier-transport layer is shared by a plurality of pixels with different emission colors.

### Example 3

This example describes the fabrication of alight-emitting device B-1 and alight-emitting device B-3 each including mmTMSPh-mDMePyPTzn, which is the organic compound of one embodiment of the present invention described in Example 1, and a comparative light-emitting device B-2, a comparative light-emitting device B-4 to a comparative light-emitting device B-6 each including a comparative organic compound, and the measurement results of the device These light-emitting devices emit blue fluorescent light. Structural formulae of organic compounds used for the light-emitting devices are shown below.

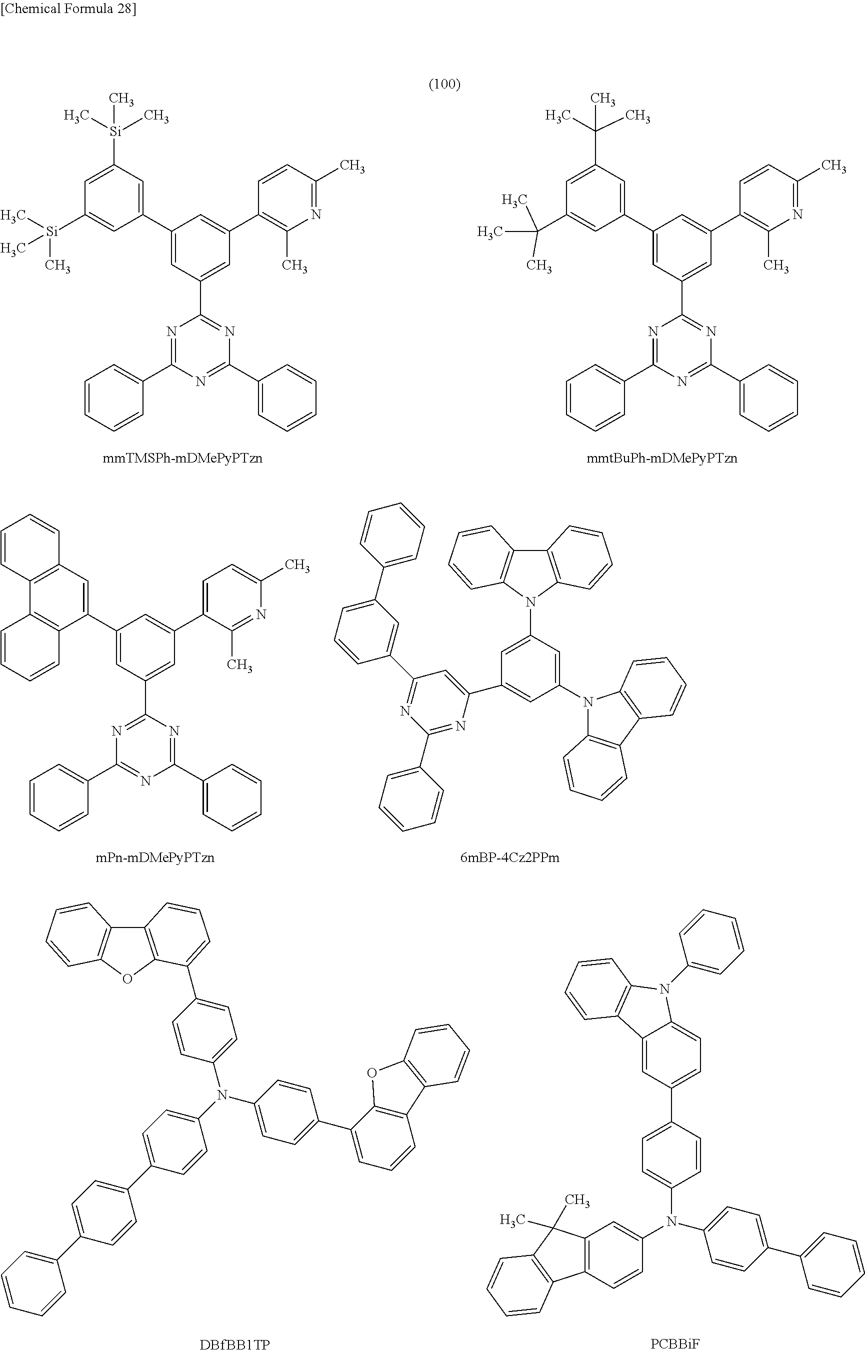

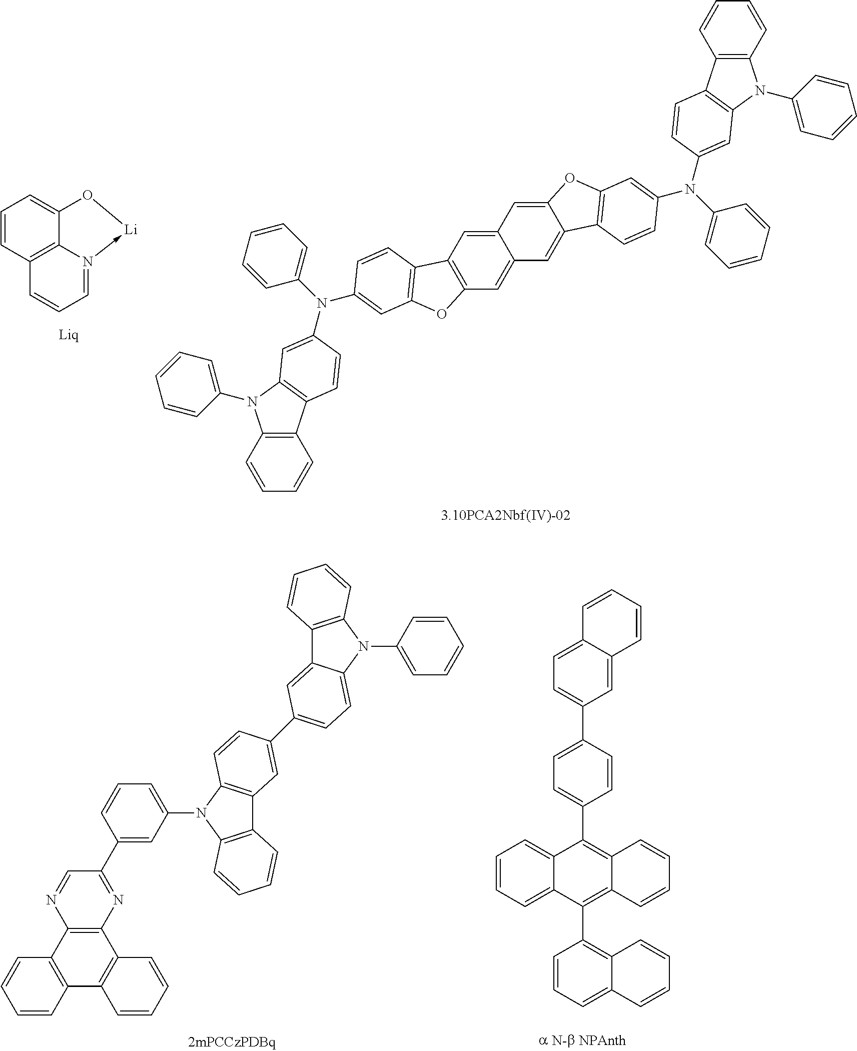

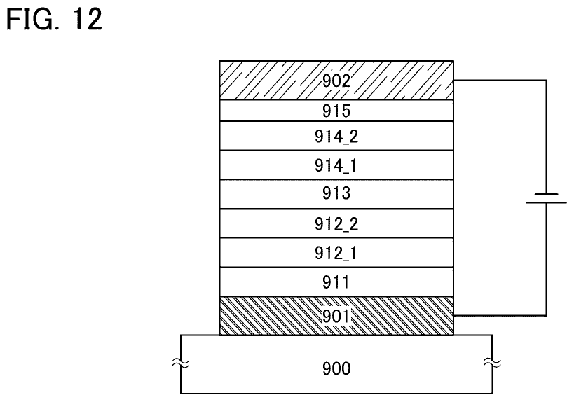

As illustrated in FIG. 12, the light-emitting devices each have a structure in which a hole-injection layer 911, hole-transport layers (a first hole-transport layer 912_1 and a second hole-transport layer 9122), a light-emitting layer 913, electron-transport layers (a first electron-transport layer 914_1 and a second electron-transport layer 9142), and an electron-injection layer 915 are stacked in this order over a first electrode 901 formed over a glass substrate 900.

### <Fabrication Method of Light-Emitting Device B-1>

Indium tin oxide containing silicon oxide (ITSO) was deposited by a sputtering method over the glass substrate 900 to a thickness of 55 nm, so that the first electrode 901 as a transparent electrode was formed. The electrode area was set to 4 mm² (2 mm×2 mm).

Next, as in the fabrication method of the light-emitting device G-1 described in Example 2, pretreatment for fabricating the light-emitting device over a substrate and vacuum baking were performed. After that, natural cooling was performed.

Next, after the hole-injection layer 911 was formed as in the fabrication method of the light-emitting device G-1 in Example 2, the first hole-transport layer 912_1 was formed to a thickness of 25 nm over the hole-injection layer 911 by evaporation of PCBBiF. Then, the second hole-transport layer 912_2 was formed to a thickness of 10 nm by evaporation of N,N-bis[4-(dibenzofuran-4-yl)phenyl]-4-amino-p-terphenyl (abbreviation: DBfBB1TP).

Next, over the second hole-transport layer 912_2, 9-(1-naphthyl)-10-[4-(2-naphthyl)phenyl]anthracene (abbreviation: αN-βNPAnth) and N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02) were deposited by co-evaporation to a thickness of 25 nm at the weight ratio of 1:0.015 (αN-PNPAnth: 3,10PCA2Nbf(IV)-02), whereby the light-emitting layer 913 was formed.

Next, the first electron-transport layer 914_1 was formed to a thickness of 10 nm over the light-emitting layer 913 by evaporation of 2mPCCzPDBq. Then, the second electron-transport layer 914_2 was formed to a thickness of 20 nm by co-evaporation of mmTMSPh-mDMePyPTzn, which is the organic compound of one embodiment of the present invention, and Liq at the weight ratio of 1:1 (mmTMSPh-mDMePyPTzn: Liq).

Next, as in the fabrication method of the light-emitting device G-1 in Example 2, the electron-injection layer 915 and the second electrode 902 were formed.

### <Fabrication Method of Comparative Light-Emitting Device B-2>

The light-emitting device B-2 is different from the light-emitting device B-1 in that mmTMSPh-mDMePyPTzn, the organic compound of one embodiment of the present invention used in the second electron-transport layer 914_2 of the light-emitting device B-1, was replaced with mmtBuPh-mDMePyPTzn, which is a comparative organic compound. Other components were fabricated in a manner similar to that for the light-emitting device B-1.

### <Fabrication Method of Light-Emitting Device B-3>

The light-emitting device B-3 is different from the light-emitting device B-1 in that the first electron-transport layer 914_1 was formed to a thickness of 10 nm by evaporation of mmTMSPh-mDMePyPTzn, which is the organic compound of one embodiment of the present invention, and that the second electron-transport layer 914_2 was formed to a thickness of 20 nm by co-evaporation of mPn-mDMePyPTzn and Liq at the weight ratio of 1:1 (mPn-mDMePyPTzn: Liq). Other components were fabricated in a manner similar to that for the light-emitting device B-1.

### <Fabrication Method of Comparative Light-Emitting Device B-4>

The comparative light-emitting device B-4 is different from the light-emitting device B-3 in that mmTMSPh-mDMePyPTzn, which is the organic compound of one embodiment of the present invention used for the first electron-transport layer 914_1, was replaced with mmtBuPh-mDMePyPTzn, which is a comparative organic compound. Other components were fabricated in a manner similar to that for the light-emitting device B-3.

### <Fabrication Method of Comparative Light-Emitting Device B-5>

The comparative light-emitting device B-5 is different from the light-emitting device B-3 in that mmTMSPh-mDMePyPTzn used in the first electron-transport layer 914_1, which is the organic compound of one embodiment of the present invention, was replaced with 6-(biphenyl-3-yl)-4-[3,5-bis(9H-carbazol-9-yl)phenyl]-2-phenylpyrimidine (abbreviation: 6mBP-4Cz2PPm), which is a comparative organic compound. Other components were fabricated in a manner similar to that for the light-emitting device B-3.

### <Fabrication Method of Comparative Light-Emitting Device B-6>

The comparative light-emitting device B-6 is different from the light-emitting device B-3 in that mmTMSPh-mDMePyPTzn, which is the organic compound of one embodiment of the present invention used for the first electron-transport layer 914_1, was replaced with 2mPCCzPDBq, which is the comparative organic compound. Other components were fabricated in a manner similar to that for the light-emitting device B-3.

Table 9 lists the device structures of the light-emitting device G-1 and the comparative light-emitting devices B-1 and B-2. Table 10 lists the device structures of the light-emitting device B-3 and the comparative light-emitting devices B-4 to B-6.

TABLE 9

| | | | | Light-emitting | Comparative light- |
|---|---|---|---|---|---|
| | | Thickness | | device B-1 | emitting device B-2 |
| Second electrode | | 100 | nm | Al | |
| Electron-injection layer | | 1 | nm | LiF | |
| Electron- | 2 | 20 | nm | mmTMSPh-mDMePyPTzn:Liq (1:1) | mmtBuPh-mDMePyPTzn:Liq (1:1) |

| transport layer | 1 | 10 | nm | 2mPCCzPDBq |
|---|---|---|---|---|
| Light-emitting layer | | 25 | nm | αN-βNPAnth:3,10PCA2Nbf(IV)-02 (1:0.015) |
| Hole-transport | 1 | 10 | nm | DBfBB1TP |
| layer | 2 | 25 | nm | PCBBiF |

| | | | |
|---|---|---|---|
| Hole-injection layer | 10 | nm | PCBBiF:OCHD-003 (1:0.03) |
| First electrode | 55 | nm | ITSO |

TABLE 10

| | | | | Comparative | Comparative | Comparative |
|---|---|---|---|---|---|---|
| | | | Light-emitting | light-emitting | light-emitting | light-emitting |
| | | Thickness | device B-3 | device B-4 | device B-5 | device B-6 |
| Second | | 100 nm | Al | | | |
| electrode | | | | | | |
| Electron- | | 1 nm | LiF | | | |
| injection | | | | | | |
| layer | | | | | | |
| Electron- | 2 | 20 nm | mPn-mDMePyPTzn:Liq (1:1) | | | |
| transport | 1 | 10 nm | mmTMSPh- | mmtBuPh- | 6mBP- | 2mPCCzPDBq |
| layer | | | mDMePyPTzn | mDMePyPTzn | 4Cz2PPm | |

| Light-emitting | | 25 nm | αN-βNPAnth:3,10PCA2Nbf(IV)-02 (1:0.015) |
|---|---|---|---|
| layer | | | |
| Hole- | 1 | 10 nm | DBfBB1TP |
| transport | 2 | 25 nm | PCBBiF |
| layer | | | |

| | | |
|---|---|---|
| Hole-injection | 10 nm | PCBBiF:OCHD-003 (1:0.03) |
| layer | | |
| First electrode | 55 nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for one hour were performed at the time of sealing). Then, the characteristics of the light-emitting devices were measured.

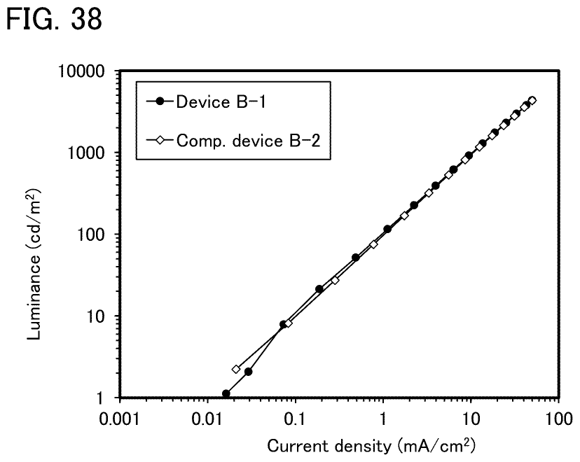

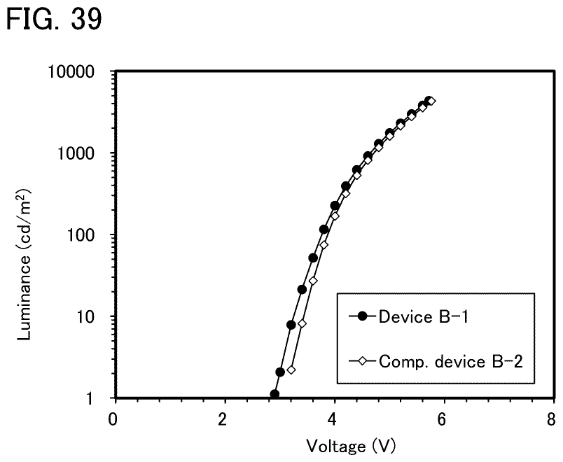

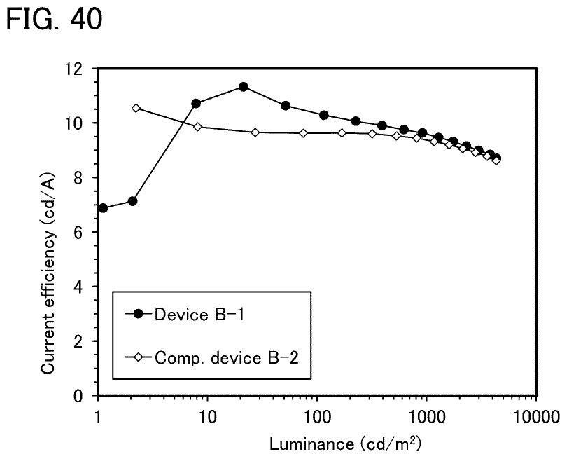

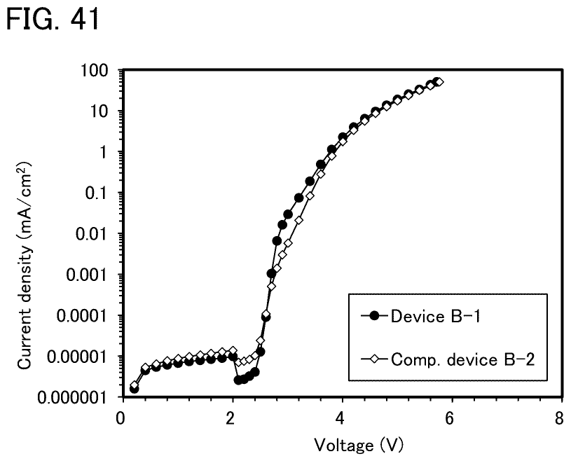

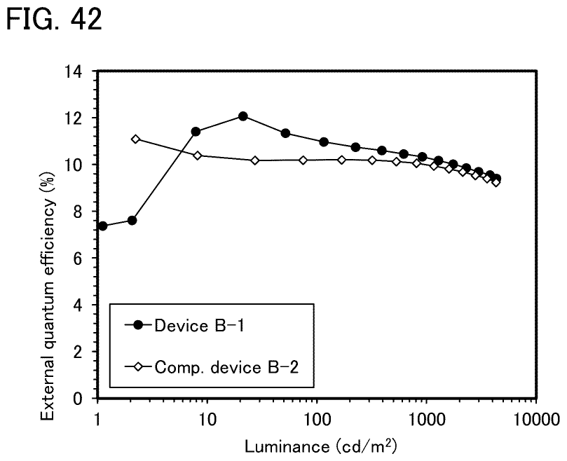

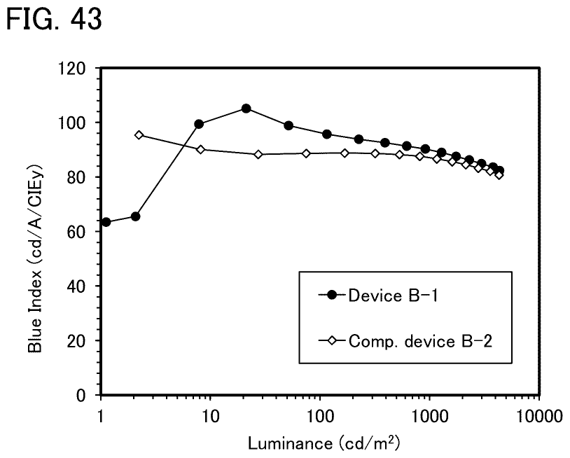

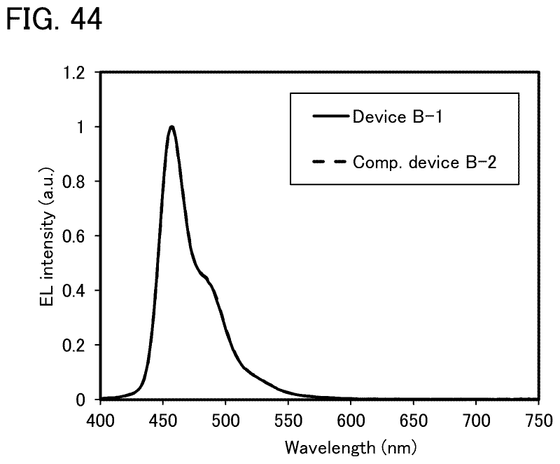

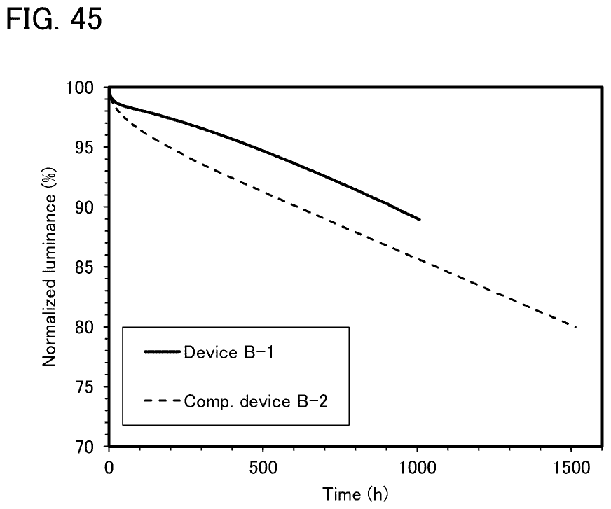

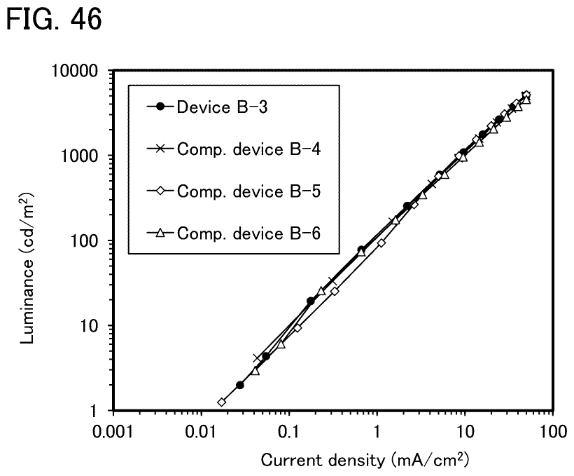

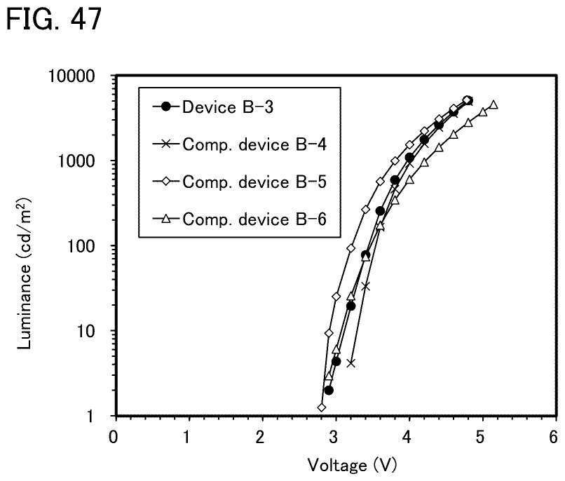

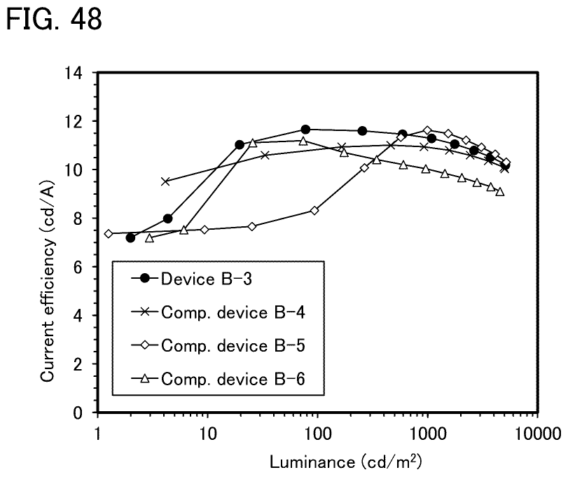

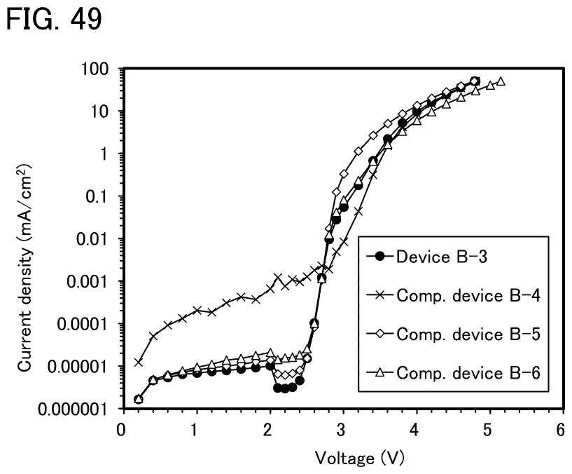

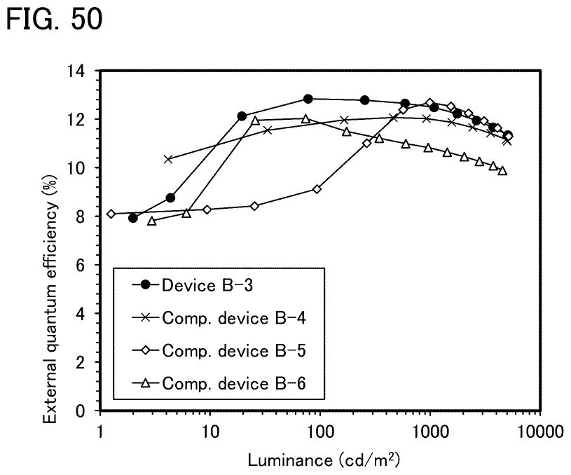

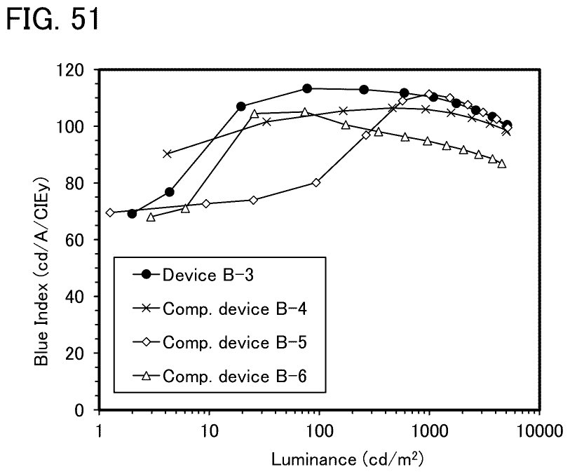

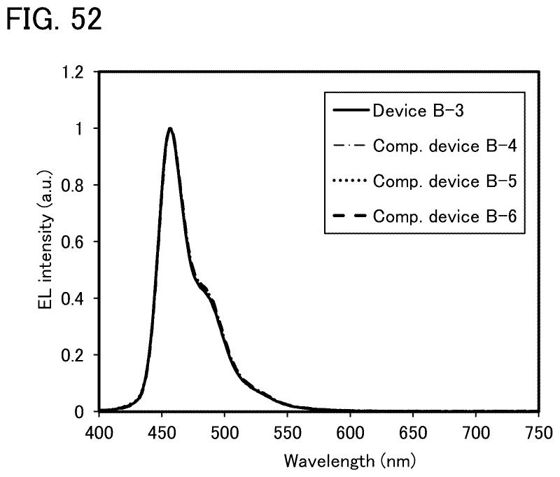

FIG. 38 shows luminance-current density characteristics of the light-emitting device B3-1 and the comparative light-emitting device B-2. FIG. 39 shows luminance-voltage characteristics thereof. FIG. 40 shows current efficiency-luminance characteristics thereof. FIG. 41 shows current density-voltage characteristics thereof. FIG. 42 shows external quantum efficiency-luminance characteristics thereof. FIG. 43 shows blue index-luminance characteristics thereof. FIG. 44 shows electroluminescence spectra thereof. FIG. 45 shows luminance changes over driving time when light-emitting device B-1 and the comparative light-emitting device B-2 are driven at a constant current of 2 mA (50 mA/cm²). FIG. 46 shows luminance-current density characteristics of the light-emitting device B-3 and the comparative light-emitting devices B-4 to B-6. FIG. 47 shows luminance-voltage characteristics thereof. FIG. 48 shows current efficiency-luminance characteristics thereof. FIG. 49 shows current density-voltage characteristics thereof. FIG. 50 shows external quantum efficiency-luminance characteristics thereof. FIG. 51 shows blue index-luminance characteristics thereof. FIG. 52 shows electroluminescence spectra thereof. In the legends in FIG. 38 to FIG. 52, the light-emitting device B-1, the comparative light-emitting device B-2, the light-emitting device B-3, the comparative light-emitting device B-4, the comparative light-emitting device B-5, and the comparative light-emitting device B-6 are denoted by Device B-1, Comp. device B-2, Device B-3, Comp. device B-4, Comp. device B-5, and Comp. device B-6, respectively.

Note that the blue index (BI) is a value obtained by dividing current efficiency (cd/A) by the y value of CIE chromaticity (x, y), and is one of the indicators of characteristics of blue light emission. As the y chromaticity value of blue light emission becomes smaller, the color purity thereof tends to be higher. Blue light emission having a small y chromaticity value and high color purity enables expression of blue with a wide range of chromaticity on a display and reduces luminance of blue light emission necessary for a display to express white, leading to lower power consumption of a display. Thus, the BI, which is current efficiency based on the y chromaticity value as one of the indicators of color purity of blue, is suitably used as a means for showing efficiency of blue light emission in some cases. The light-emitting device with a higher BI can be regarded as a blue-light-emitting device having higher efficiency for a display.

Table 11 shows the main characteristics of the light-emitting devices at a luminance of approximately 100 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 11

| | | | | | | | | External | |
|---|---|---|---|---|---|---|---|---|---|
| | | | Current | | | | Current | quantum | |
| | Voltage | Current | density | Chromaticity | Chromaticity | Luminance | efficiency | efficiency | BI |
| | (V) | (mA) | (mA/cm²) | x | y | (cd/m²) | (cd/A) | (%) | (cd/A/CIEy) |
| Light-emitting | 3.80 | 0.0450 | 1.12 | 0.137 | 0.107 | 116 | 10.3 | 11.0 | 95.7 |
| device B-1 | | | | | | | | | |
| Comparative | 3.80 | 0.0311 | 0.778 | 0.137 | 0.109 | 74.9 | 9.63 | 10.2 | 88.6 |
| light-emitting | | | | | | | | | |
| device B-2 | | | | | | | | | |
| Light-emitting | 3.40 | 0.0266 | 0.666 | 0.138 | 0.103 | 77.6 | 11.7 | 12.8 | 113 |
| device B-3 | | | | | | | | | |
| Comparative | 3.60 | 0.0604 | 1.51 | 0.137 | 0.104 | 165 | 10.9 | 12.0 | 105 |
| light-emitting | | | | | | | | | |
| device B-4 | | | | | | | | | |
| Comparative | 3.20 | 0.0449 | 1.12 | 0.138 | 0.104 | 93.3 | 8.32 | 9.12 | 80.1 |
| light-emitting | | | | | | | | | |
| device B-5 | | | | | | | | | |
| Comparative | 3.40 | 0.0264 | 0.659 | 0.137 | 0.107 | 73.8 | 11.2 | 12.0 | 105 |
| light-emitting | | | | | | | | | |
| device B-6 | | | | | | | | | |

From FIG. 38 to FIG. 52 and Table 11, the light-emitting device B-1 was found to be a light-emitting device with favorable characteristics that emits blue light derived from 3,10PCA2Nbf(IV)-02.

As shown in FIG. 39 and FIG. 41, the driving voltage of the light-emitting device B-1 was lower than that of the comparative light-emitting device B-2 at low luminance or low current density. As shown in FIG. 40, FIG. 42, and FIG. 43, the light-emitting device B-1 has higher current efficiency, higher external quantum efficiency, and a higher blue index than the comparative light-emitting device B-2.

As shown in FIG. 48, FIG. 50, and FIG. 51, the light-emitting device B-3 has higher current efficiency, higher external quantum efficiency, and a higher blue index than the comparative light-emitting devices B-4 to B-6 in a wide luminance range.

Here, Table 12 shows the ordinary refractive indices (nₒ) and GSP slopes of the evaporated films of major organic compounds used in the light-emitting devices. The ordinary refractive indices at a wavelength of 633 nm are shown as the ordinary refractive indices in Table 12. A spectroscopic ellipsometer (M-2000U, manufactured by J.A. Woollam Japan) was used to measure the ordinary refractive indices, and the material for each layer was deposited to a thickness of 50 nm over a quartz substrate by a vacuum evaporation method to form films used as measurement samples. The GSP slopes of the films of the organic compounds in Table 12 were measured by the method described in Embodiment 1.

TABLE 12

| | nₒ (@633 nm) | GSP slope (mV/nm) |
|---|---|---|
| 2mPCCzPDBq | 1.88 | 12.4 |
| 6mBP-4Cz2PPm | 1.76 | 21.5 |
| mmtBuPh-mDMePyPTzn | 1.62 | 44.3 |
| mmTMSPh-mDMePyPTzn | 1.60 | 35.0 |

As shown in Table 12, mmTMSPh-mDMePyPTzn, which is the organic compound of one embodiment of the present invention, is an organic compound with a lower refractive index of the film than 6mBP-4Cz2PPm and 2mPCCzPDBq and a lower GSP slope of the film than mmtBuPh-mDMePyPTzn.

The above results reveal that the light-emitting device using the organic compound of one embodiment of the present invention has a low driving voltage and high emission efficiency. This is because, in the light-emitting device, the organic compound of one embodiment of the present invention in the electron-transport layer enables higher efficiency of light extraction from the light-emitting layer and effective voltage application to the light-emitting layer owing to the low refractive index and the small GSP slope of the film of the organic compound of one embodiment of the present invention.

### Example 4

### Synthesis Example 2

Described in this synthesis example are a method for synthesizing the organic compound of one embodiment of the present invention, 2-{3′,5′-bis(trimethylsilyl)-5-(pyrimidin-5-yl)biphenyl-3-yl}-4,6-diphenyl-1,3,5-triazine (abbreviation: mmTMSPh-mPmPTzn) (Structural Formula (102)), and the physical properties thereof.

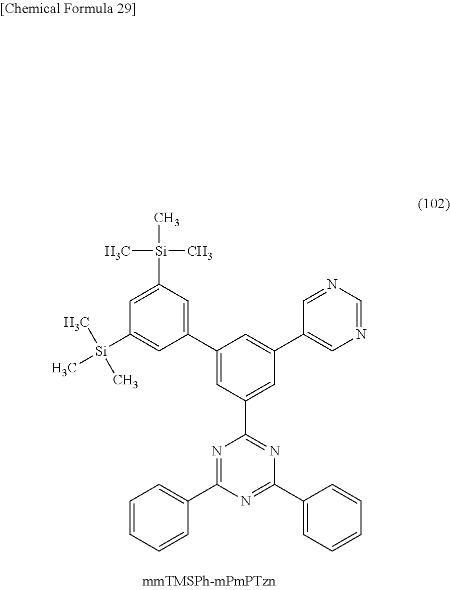 [SMILES: C[Si](C)(C)c1cc(-c2cc(-c3cncnc3)cc(-c3nc(-c4ccccc4)nc(-c4ccccc4)n3)c2)cc([Si](C)(C)C)c1]

### Step 1: Synthesis of 2-{5-chloro-[3′,5′-bis(trimethylsilyl)]biphenyl-3-yl}-4,6-diphenyl-1,3,5-triazine

Into a three-neck flask were put 7.5 g (18 mmol) of 2-(3-bromo-5-chlorophenyl)-4,6-diphenyl-1,3,5-triazine, 6.8 g (20 mmol) of 2-[3,5-bis(trimethylsilyl)phenyl]-4,4,5,5-tetramethyl-1,3,2-dioxaborolane, 4.9 g (36 mmol) of potassium carbonate, 70 mL of toluene, 35 mL of ethanol (abbreviation: EtOH), and 18 mL of water, and the mixture was degassed. After the degassing, 40 mg (0.18 mmol) of palladium(II) acetate (abbreviation: Pd(OAc)₂) and 0.22 g (0.71 mmol) of tris(2-methylphenyl)phosphine (abbreviation: P(o-tolyl)₃) were added thereto, and the mixture was stirred at 80° C. for 11 hours. After the reaction was completed, toluene and water were added to this mixture, and the mixture was separated into an organic layer and an aqueous layer. An aqueous layer was subjected to extraction with toluene, the extracted solution and the organic layer were combined, and magnesium sulfate was added thereto to adsorb moisture. This mixture was gravity-filtered, and the obtained filtrate was concentrated to give a pale orange solid. This solid was purified by silica gel column chromatography with a developing solvent of toluene and hexane in a ratio of 1:10. Then, 10.4 g of a white solid containing a target substance was obtained. The synthesis scheme of Step 1 is shown by (a-3).

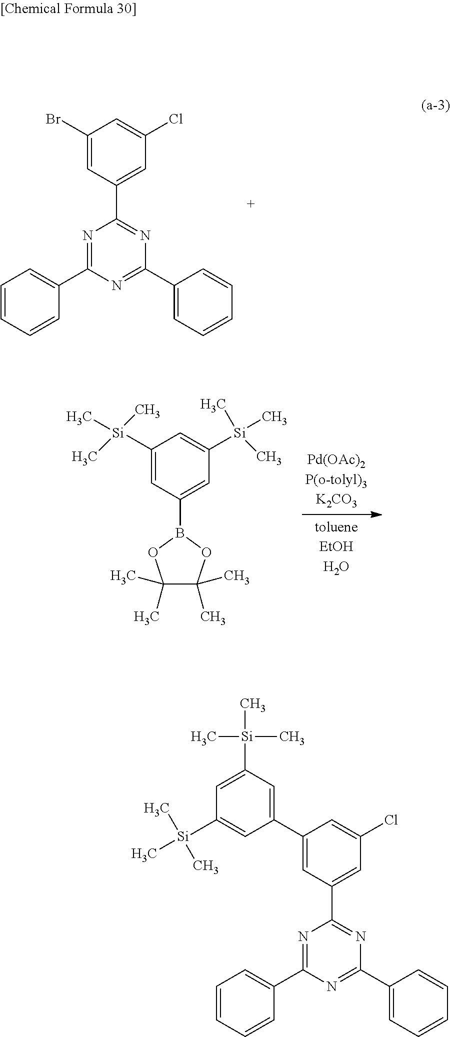

### Step 2: Synthesis of mmTMSPh-mPmPTzn

Into a three-neck flask were put 5.0 g (8.8 mmol) of 2-{5-chloro-[3′,5′-bis(trimethylsilyl)]biphenyl-3-yl}-4,6-diphenyl-1,3,5-triazine obtained in Step 1, 2.2 g (18 mmol) of 5-pyrimidylboronic acid, 3.7 g (27 mmol) of potassium carbonate, 50 mL of tetrahydrofuran (abbreviation: THF), and 13 mL of water, and the mixture was degassed. After the degassing, 40 mg (0.18 mmol) of palladium(II) acetate (abbreviation: Pd(OAc)₂) and 0.17 g (0.35 mmol) of 2-dicyclohexylphosphino-2′,4′,6′-triisopropylbiphenyl (abbreviation: XPhos) were added thereto, and the mixture was stirred at 65° C. for 18 hours. After the reaction was completed, toluene and water were added to this mixture, and the mixture was separated into an organic layer and an aqueous layer. An aqueous layer was subjected to extraction with toluene, the extracted solution and the organic layer were combined, and magnesium sulfate was added thereto to adsorb moisture. This mixture was gravity-filtered, and the obtained filtrate was concentrated to give a yellow solid. This solid was purified by silica gel column chromatography with a developing solvent of toluene and ethyl acetate in a ratio of 10:1, which was then changed to 5:1, giving a pale yellow solid. This solid was recrystallized with toluene and ethanol to give 2.6 g of a target white solid in a yield of 49%. By a train sublimation method, 2.6 g of the obtained solid was purified. In the purification by sublimation, the solid was heated at 260° C. under a pressure of 3.3 Pa for 2 hours. After the sublimation purification, 1.1 g of a white solid was obtained at a yield of 44%. The synthesis scheme of Step 2 is shown in (a-4) below.

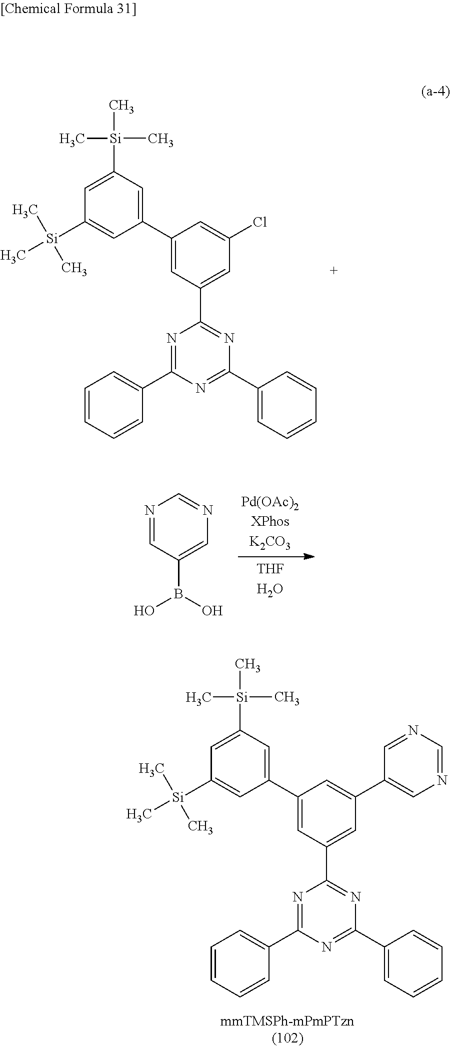 [SMILES: C[Si](C)(C)c1cc(-c2cc(Cl)cc(-c3nc(-c4ccccc4)nc(-c4ccccc4)n3)c2)cc([Si](C)(C)C)c1 ; OB(O)c1cncnc1 ; C[Si](C)(C)c1cc(-c2cc(-c3cncnc3)cc(-c3nc(-c4ccccc4)nc(-c4ccccc4)n3)c2)cc([Si](C)(C)C)c1]

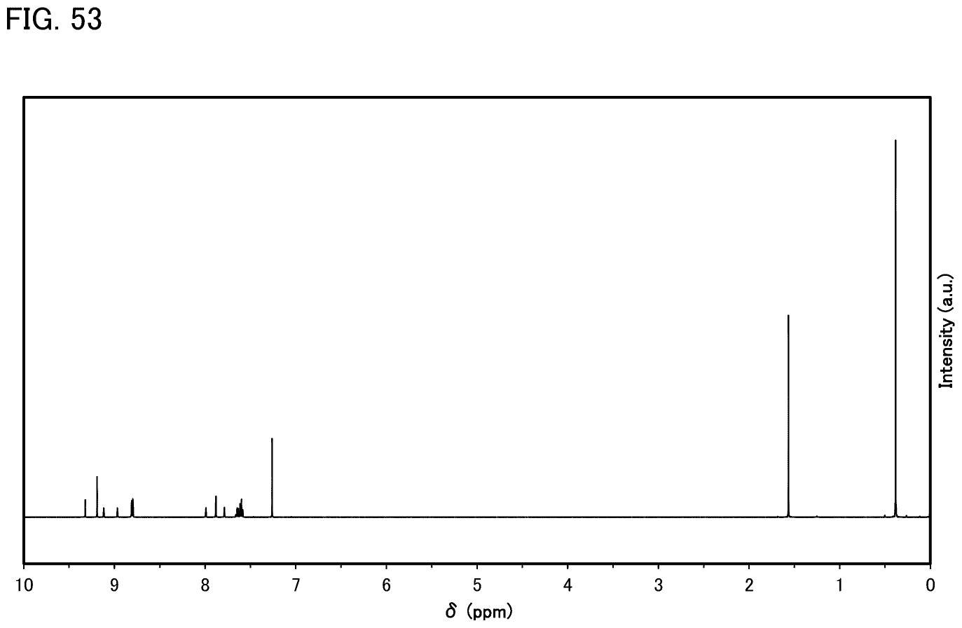

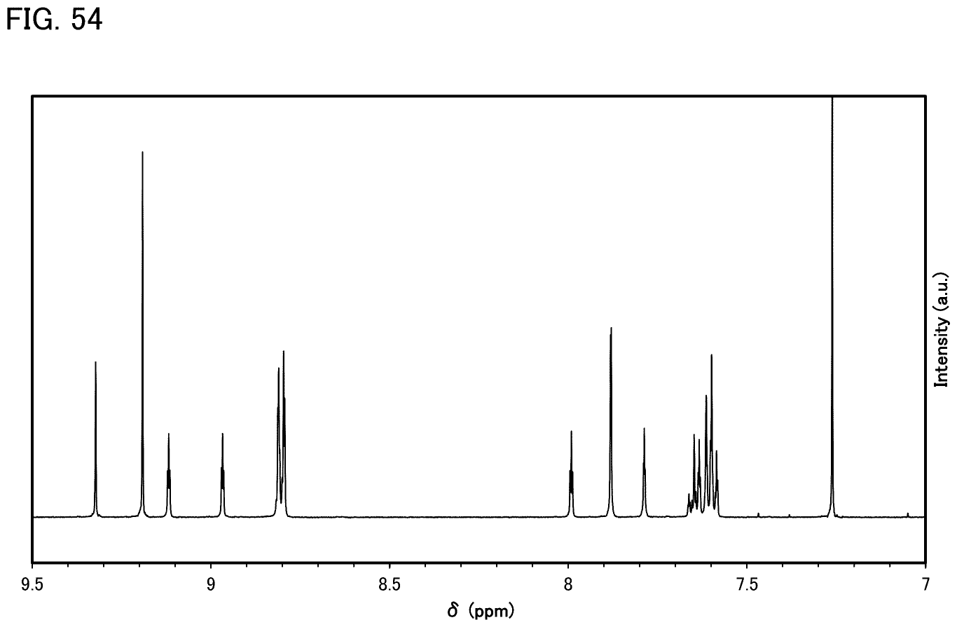

Analysis results by nuclear magnetic resonance (¹H-NMR) spectroscopy of the white solid obtained in the above Step 2 are shown below. FIG. 53 is the ¹H-NMR chart, and FIG. 54 is an enlarged view of the range of 7 ppm to 9.5 ppm of FIG. 53. These results revealed that the organic compound of one embodiment of the present invention, mmTMSPh-mPmPTzn, was obtained in this example.

¹H-NMR. δ (CDCl₃, 500 MHz): 0.38 (s, 18H), 7.58-7.66 (m, 6H), 7.79 (t, 1H, J=1.1 Hz), 7.88 (d, 2H, J=1.1 Hz), 7.99 (t, 1H, J=1.7 Hz), 8.79-8.82 (m, 4H), 8.97 (t, 1H, J=1.6 Hz), 9.12 (t, 1H, J=1.6 Hz), 9.19 (s, 2H), 9.32 (s, 1H).

### <Measurement of Emission and Absorption Spectra>

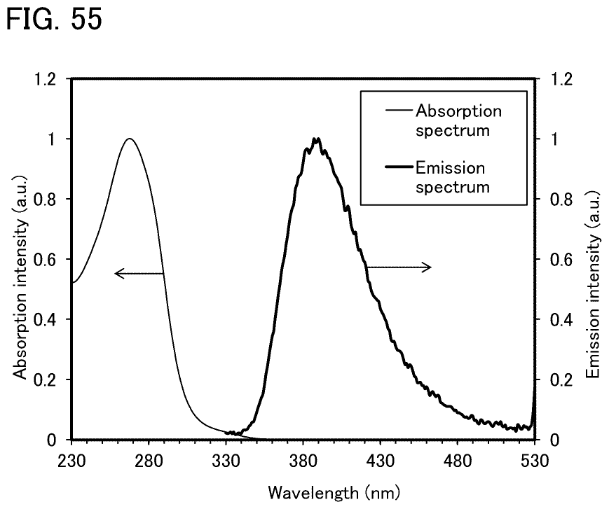

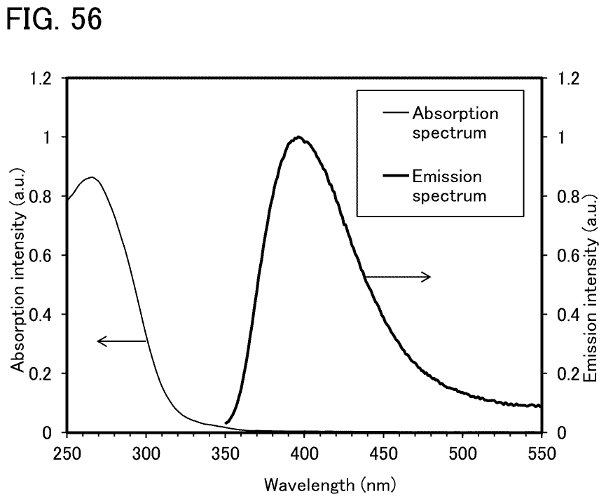

FIG. 55 shows the measurement results of an absorption spectrum and an emission spectrum of a dichloromethane solution of mmTMSPh-mPmPTzn, as in <Measurement of emission and absorption spectra>in Example 1. Furthermore, the absorption and emission spectra of the thin film are shown in FIG. 56.

As shown in FIG. 55, the dichloromethane solution of mmTMSPh-mPmPTzn has an absorption spectrum peak at a wavelength of 270 nm and an emission spectrum peak at a wavelength of 390 nm (excitation wavelength: 270 nm). As shown in FIG. 56, the thin film of mmTMSPh-mPmPTzn has an absorption spectrum peak at a wavelength of 266 nm and an emission spectrum peak at a wavelength of 396 nm (excitation wavelength: 300 nm). It was found from FIG. 55 and FIG. 56 that mmTMSPh-mPmPTzn exhibits no absorption within the visible range (at wavelengths longer than 450 nm).

### <Measurement of Refractive Index>

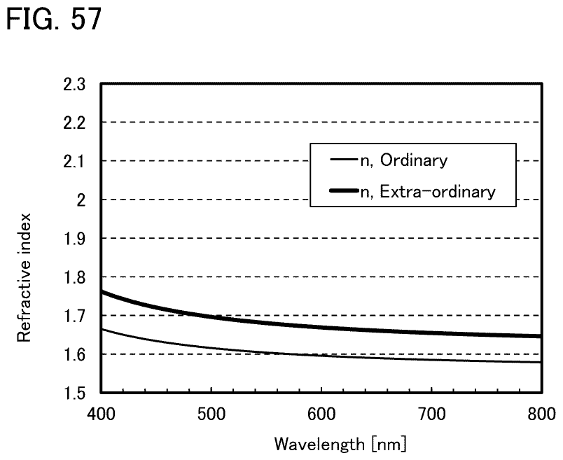

FIG. 57 shows the measurement results of the refractive indices of the film of mmTMSPh-mPmPTzn, as in <Measurement of refractive index in Example 1>. As shown in FIG. 57, the film of mmTMSPh-mPmPTzn has an ordinary refractive index in the range of 1.50 to 1.75 for the entire blue emission region (at wavelengths of 455 nm and 465 nm) and also has an ordinary refractive index at a wavelength of 633 nm in the range of 1.45 to 1.70, indicating that the film has a low refractive index.

### <Measurement of GSP Slope and Pz>

Next, the GSP slope of an evaporated film of mmTMSPh-mPmPTzn was measured. The measurement was performed by the method described in Embodiment 1. Furthermore, the degree of SOP (Pz) of the evaporated film of mmTMSPh-mPmPTzn in the direction perpendicular to the substrate surface was calculated. Note that Pz is a value obtained by multiplying the GSP slope and the relative permittivity of the film. As the dielectric constant of the film, it is possible to use a value obtained by multiplying the vacuum permittivity and the square of the ordinary refractive index nₒ (the value at a wavelength of 633 nm). Table 13 shows the measurement results. Table 13 also shows the GSP slope and the Pz of an evaporated film of 2-{3-(3,5-di-tert-butyl)phenyl-5-(pyrimidin-5-yl)phenyl}-4,6-diphenyl-1,3,5-triazine (abbreviation: mmtBuPh-mPmPTzn) for comparison. The chemical formula of mmTMSPh-mPmPTzn, which is the organic compound of one embodiment of the present invention, and the chemical formula of mmtBuPh-mPmPTzn, which is the comparative organic compound, are shown below.

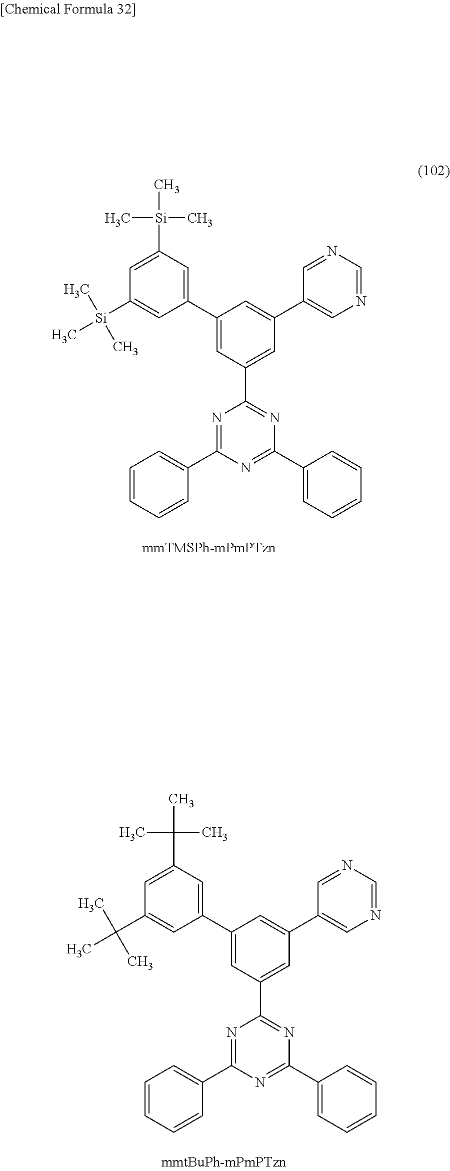 [SMILES: C[Si](C)(C)c1cc(-c2cc(-c3cncnc3)cc(-c3nc(-c4ccccc4)nc(-c4ccccc4)n3)c2)cc([Si](C)(C)C)c1 ; CC(C)(C)c1cc(-c2cc(-c3cncnc3)cc(-c3nc(-c4ccccc4)nc(-c4ccccc4)n3)c2)cc(C(C)(C)C)c1]

TABLE 13

| | GSP slope (mV/nm) | Pz (mC/m²) |
|---|---|---|
| mmTMSPh-mDMePyPTzn | 64.3 | 1.44 |
| mmtBuPh-mPmPTzn | 103.7 | 2.38 |

As shown in Table 13, mmTMSPh-mPmPTzn, which is the organic compound of one embodiment of the present invention, has a smaller GSP slope and Pz in the form of a film than the film than mmtBuPh-mPmPTzn, which is the comparative organic compound. In mmTMSPh-mPmPTzn, the trisubstituted silyl groups have a structure in which the quaternary carbon atoms of the two tert-butyl groups of mmtBuPh-mDMePyPTzn are each replaced with a silicon atom. It can be considered that, since a silicon atom has lower electronegativity than a carbon atom, mmTMSPh-mPmPTzn had a smaller permanent electric dipole moment than mmtBuPh-mDMePyPTzn, which resulted in the small Pz and GSP slope in the evaporated film.

### Example 5

This example describes the fabrication of a light-emitting device G-6 including mmTMSPh-mPmPTzn, which is the organic compound of one embodiment of the present invention described in Example 4, and a comparative light-emitting device G-7 and a comparative light-emitting device G-8 each including a comparative organic compound, and the measurement results of the device. These light-emitting devices emit green phosphorescent light. Structural formulae of organic compounds used for the light-emitting devices are shown below.

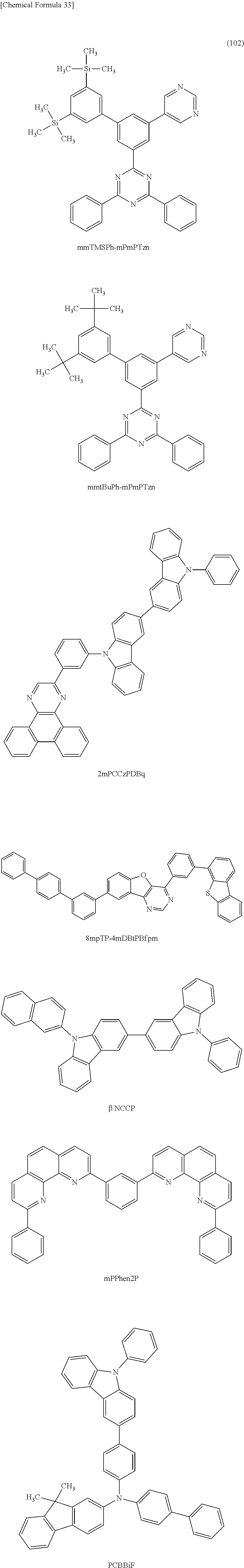

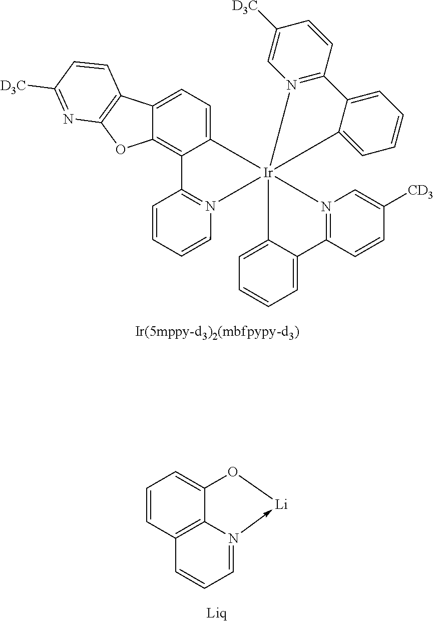

### <Fabrication Method of Light-Emitting Device G-6>

The light-emitting device G-6 is different from the light-emitting device G-1 in that the first electron-transport layer 914_1 was formed to a thickness of 20 nm by evaporation of mmTMSPh-mPmPTzn, which is the organic compound of one embodiment of the present invention, and that the second electron-transport layer 914_2 was formed to a thickness of 20 nm by evaporation of mPPhen2P. Other components were fabricated in a manner similar to that for the light-emitting device G-1.

### <Fabrication Method of Comparative Light-Emitting Device G-7>

The comparative light-emitting device G-7 is different from the light-emitting device G-6 in that mmTMSPh-mPmPTzn, which is the organic compound of one embodiment of the present invention used for the first electron-transport layer 914_1, is replaced with mmtBuPh-mPmPTzn, which is the comparative organic compound. Other components were fabricated in a manner similar to that for the light-emitting device G-6.

### <Fabrication Method of Comparative Light-Emitting Device G-8>

The comparative light-emitting device G-8 is different from the light-emitting device G-6 in that mmTMSPh-mPmPTzn, which is the organic compound of one embodiment of the present invention used for the first electron-transport layer 914_1, is replaced with 2mPCCzPDBq, which is the comparative organic compound. Other components were fabricated in a manner similar to that for the light-emitting device G-6.

The structures of the light-emitting device G-6 and the comparative light-emitting devices G-7 and G-8 are listed in Table 14.

TABLE 14

| | | | | Light-emitting | Comparative light- | Comparative light- |
|---|---|---|---|---|---|---|
| | | Thickness | | device G-6 | emitting device G-7 | emitting device G-8 |
| Seconde lectrode | | 100 | nm | Al | | |
| Electron-injection layer | | 1 | nm | LiF | | |
| Electron | 2 | 20 | nm | mPPhen2P | | |
| transport layer | 1 | 20 | nm | mmTMSPh-mPmPTzn | mmtBuPh-mPmPTzn | 2mPCCzPDBq |

| | | | |
|---|---|---|---|
| Light-emitting layer | 40 | nm | 8mpTP-4mDBtPBfpm:βNCCP:Ir(5mppy-d₃)₂(mbfpypy-d₃) (0.5:0.5:0.1) |
| Hole-transport layer | 40 | nm | PCBBiF |
| Hole-injection layer | 10 | nm | PCBBiF:OCHD-003 (1:0.03) |
| First electrode | 70 | nm | ITSO |

### <Characteristics of Light-Emitting Device>

The light-emitting devices were sealed using a glass substrate in a glove box containing a nitrogen atmosphere so as not to be exposed to atmospheric air (a sealing material was applied to surround the devices, and UV treatment and heat treatment at 80° C. for one hour were performed at the time of sealing). Then, the characteristics of the light-emitting devices were measured.

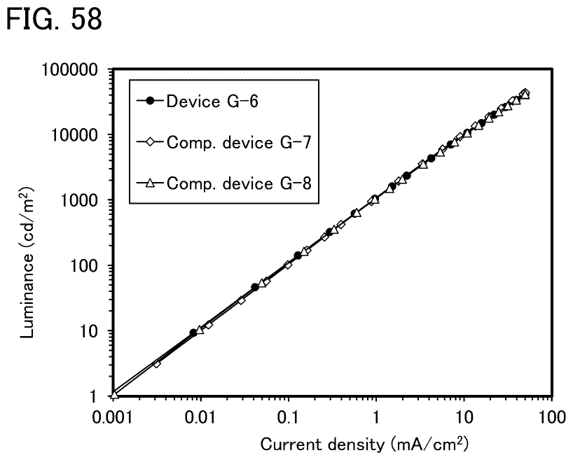

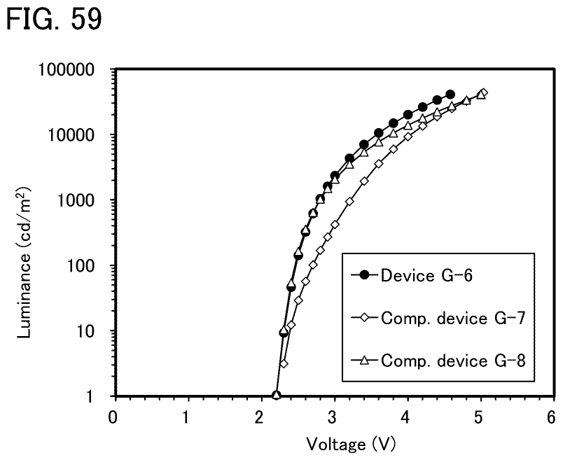

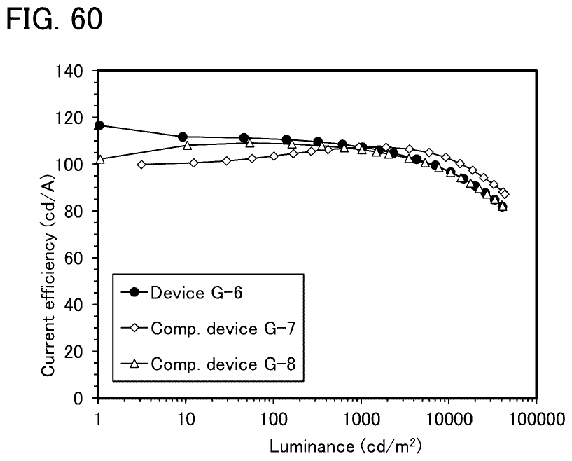

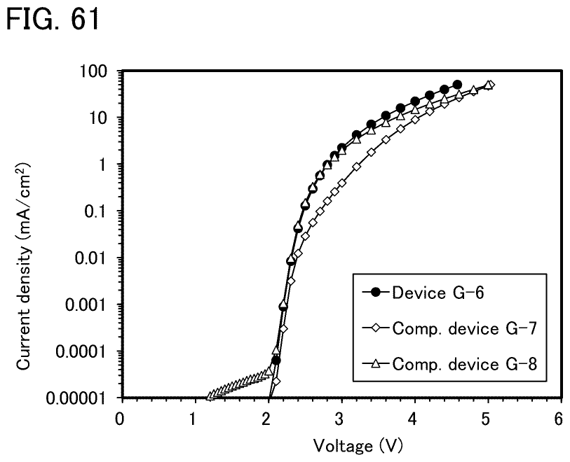

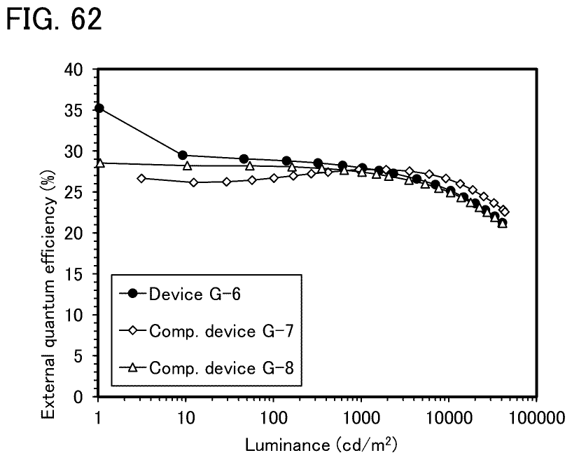

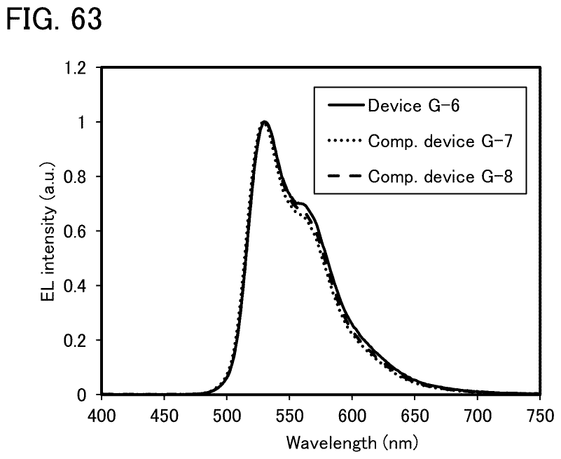

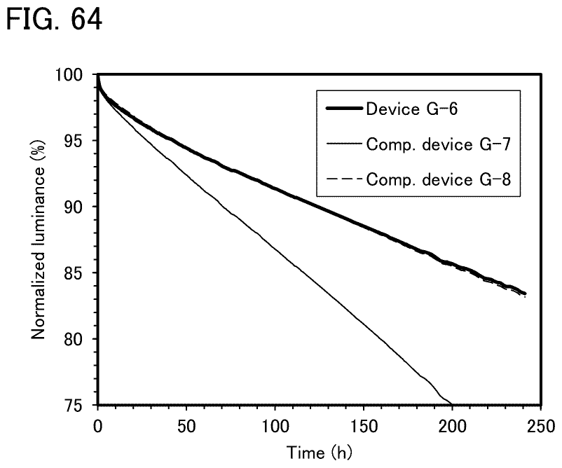

FIG. 58 shows luminance-current density characteristics of the light-emitting device G-6 and the comparative light-emitting devices G-7 and G-8. FIG. 59 shows luminance-voltage characteristics thereof. FIG. 60 shows current efficiency-luminance characteristics thereof. FIG. 61 shows current density-voltage characteristics thereof. FIG. 62 shows external quantum efficiency-luminance characteristics thereof. FIG. 63 shows electroluminescence spectra thereof. FIG. 64 shows luminance changes over driving time when the light-emitting device G-6 and the comparative light-emitting devices G-7 and G-8 are driven at a constant current of 2 mA (50 mA/cm²). In the legends in FIG. 58 to FIG. 64, the light-emitting device G-6, the comparative light-emitting device G-7, and the comparative light-emitting device G-8 are denoted by Device G-6, Comp. device G-7, and Comp. device G-8, respectively.

Table 15 shows the main characteristics of the light-emitting devices at a luminance of approximately 1000 cd/m². Note that the luminance, CIE chromaticity, and emission spectra were measured with a spectroradiometer (SR-UL1R manufactured by TOPCON TECHNOHOUSE CORPORATION). The external quantum efficiency was calculated from the luminance and the emission spectra measured with the spectroradiometer, on the assumption that the devices had Lambertian light-distribution characteristics.

TABLE 15

| | | | | | | | | External |
|---|---|---|---|---|---|---|---|---|
| | | | Current | | | | Current | quantum |
| | Voltage | Current | density | Chromaticity | Chromaticity | Luminance | efficiency | efficiency |
| | (V) | (mA) | (mA/cm²) | x | y | (cd/m²) | (cd/A) | (%) |
| Light-emitting | 2.80 | 0.0386 | 0.966 | 0.363 | 0.614 | 1036 | 107 | 27.9 |
| device G-6 | | | | | | | | |
| Comparative | 3.20 | 0.0352 | 0.879 | 0.351 | 0.623 | 944 | 107 | 27.7 |
| light-emitting | | | | | | | | |
| device G-7 | | | | | | | | |
| Comparative | 2.80 | 0.0385 | 0.963 | 0.357 | 0.619 | 1023 | 106 | 27.4 |
| light-emitting | | | | | | | | |
| device G-8 | | | | | | | | |

From FIG. 58 to FIG. 64 and Table 15, the light-emitting device G-6 was found to be a light-emitting device with favorable characteristics that emit green light derived from Ir(5mppy-d₃)₂(mbfpypy-d₃).

FIG. 59 and FIG. 61 show that the light-emitting device G-6 has a lower driving voltage than the comparative light-emitting device G-7. FIG. 60 and FIG. 62 show that the light-emitting device G-6 has higher emission efficiency than the comparative light-emitting device G-8. FIG. 64 shows that the luminance change of the light-emitting device G-6 over driving time is smaller than that of the comparative light-emitting device G-7 and is equivalent to that of the comparative light-emitting device G-8, and thus the light-emitting device G-6 has a long lifetime.

Table 16 shows the ordinary refractive indices (nₒ), Pz, and GSP slopes of the evaporated films of the organic compounds used in the first electron-transport layers 914_1 of the light-emitting devices and a film formed by co-evaporation of the same combination of organic compounds at the same mixing ratio as those in the light-emitting layer 913 of each light-emitting device. The ordinary refractive index at a wavelength of 532 nm is shown as the ordinary refractive index in Table 16. A spectroscopic ellipsometer (M-2000U, manufactured by J.A. Woollam Japan) was used to measure the ordinary refractive indices, and the material for each layer was deposited to a thickness of 50 nm over a quartz substrate by a vacuum evaporation method to form films used as measurement samples. The Pz and GSP slope of the films of the organic compounds in Table 16 were measured by the method described in Embodiment 1.

TABLE 16

| | nₒ | Pz | GSP |
|---|---|---|---|
| | (@532 | (mC/ | slope |
| | nm) | m²) | (mV/nm) |
| mmTMSPh-mDMePyPTzn | 1.61 | 1.44 | 64.3 |
| mmtBuPh-mPmPTzn | 1.63 | 2.38 | 103.7 |
| 2mPCCzPDBq | 1.92 | 0.38 | 12.4 |
| 8mpTP-4mDBtPBfpm:βNCCP:Ir(5mppy- | — | 1.67 | 51.5 |
| d₃)₂(mbfpypy-d₃) (0.5:0.5:0.1) | | | |

As shown in Table 16, the film of mmTMSPh-mPmPTzn, which is the organic compound of one embodiment of the present invention, has a lower refractive index than the films of mmtBuPh-mPmPTzn and 2mPCCzPDBq, which are comparative organic compounds, and has a smaller Pz and a smaller GSP slope than the film of mmtBuPh-mPmPTzn. As shown in Table 16, the film of mmTMSPh-mPmPTzn, which is the organic compound of one embodiment of the present invention, also has a smaller Pz than the film formed by co-evaporation of the same combination of organic compounds at the same mixing ratio as those in the light-emitting layer 913. Meanwhile, the film of mmtBuPh-mPmPTzn, which is a comparative organic compound, has a larger Pz than the film formed by co-evaporation of the same combination of organic compounds at the same mixing ratio as those in the light-emitting layer 913.

As described above, the light-emitting device G-6 has higher emission efficiency than the comparative light-emitting device G-8. This result can be explained by the fact that the film of mmTMSPh-mPmPTzn used in the first electron-transport layer 914_1 of the light-emitting device G-6 has a lower refractive index than the film of 2mPCCzPDBq used in the first electron-transport layer 914_1 of the comparative light-emitting device G-8, as shown in Table 16. In other words, the light-emitting device G-6 achieved higher light extraction efficiency owing to the use of mmTMSPh-mPmPTzn, which has a low refractive index in the form of a film, in the first electron-transport layer 914_1, resulting in increased light emission efficiency.

As described above, the light-emitting device G-6 has a lower driving voltage and a longer lifetime than the comparative light-emitting device G-7. This result can be explained by the fact that, as shown in Table 16, the film of mmTMSPh-mPmPTzn used as the first electron-transport layer 914_1 of the light-emitting device G-6 has a smaller Pz than the film formed by co-evaporation of the same combination of organic compounds at the same mixing ratio as those in the light-emitting layer 913, whereas the film of mmtBuPh-mPmPTzn used in the comparative light-emitting device G-7 has a larger Pz than the film formed by co-evaporation of the same combination of organic compounds at the same mixing ratio as those in the light-emitting layer 913. In other words, the light-emitting device G-6 enabled virtual positive interface charge to be generated at the interface between the light-emitting layer 913 and the first electron-transport layer 914_1 owing to the use of mmTMSPh-mPmPTzn having a smaller Pz in the form of a film than the light-emitting layer 913, which achieved improved property of electron injection from the first electron-transport layer 914_1 into the light-emitting layer 913. Furthermore, the light-emitting device had a favorable carrier balance and a longer lifetime.

This application is based on Japanese Patent Application Serial No. 2024-230213 filed with Japan Patent Office on Dec. 26, 2024, the entire contents of which are hereby incorporated by reference.

What is claimed is:

1. An organic compound represented by General Formula (G1): 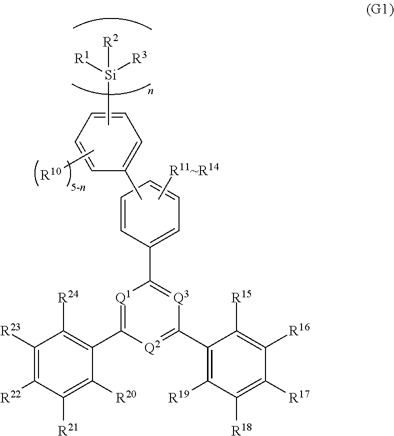
wherein:
each of Q¹ to Q³ independently represents N or CH;
at least two of Q¹ to Q³ represent N;
each of R¹ to R³ independently represents an alkyl group having 1 to 6 carbon atoms or a phenyl group;
n is an integer greater than or equal to 2 and less than or equal to 5;
R¹⁰ represents hydrogen, an alkyl group having 1 to 6 carbon atoms, or a cycloalkyl group having 3 to 10 carbon atoms;
each of R¹¹ to R²⁴ independently represents any one of hydrogen, an alkyl group having 1 to 6 carbon atoms, a cycloalkyl group having 3 to 10 carbon atoms, a substituted or unsubstituted aryl group having 6 to 30 carbon atoms, and a substituted or unsubstituted heteroaryl group having 3 to 30 carbon atoms;
at least any one of R¹¹ to R²⁴ represents a substituted or unsubstituted heteroaryl group having 3 to 30 carbon atoms;
a plurality of R¹'s are the same or different from each other;
a plurality of R²'s are the same or different from each other;
a plurality of R³'s are the same or different from each other; and
when 5-n is greater than or equal to 2, a plurality of R¹⁰'s are the same or different from each other.

2. The organic compound according to claim 1,
wherein the organic compound is represented by General Formula (G2): 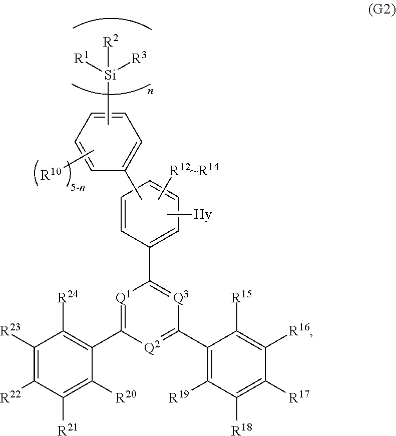
wherein:
each of R¹² to R²⁴ independently represents any one of hydrogen, an alkyl group having 1 to 6 carbon atoms, a cycloalkyl group having 3 to 10 carbon atoms, and a substituted or unsubstituted aryl group having 6 to 30 carbon atoms; and
Hy represents a substituted or unsubstituted heteroaryl group having 3 to 30 carbon atoms.

3. The organic compound according to claim 1,
wherein the substituted or unsubstituted heteroaryl group having 3 to 30 carbon atoms comprises nitrogen.

4. The organic compound according to claim 1,
wherein one or more of atoms included in an aromatic ring of the substituted or unsubstituted heteroaryl group having 3 to 30 carbon atoms are nitrogen atoms.

5. The organic compound according to claim 1,
wherein the substituted or unsubstituted heteroaryl group having 3 to 30 carbon atoms is any one of a substituted or unsubstituted pyridinyl group, a substituted or unsubstituted pyrimidinyl group, and a substituted or unsubstituted pyrazinyl group.

6. The organic compound according to claim 5,
wherein the substituted or unsubstituted heteroaryl group having 3 to 30 carbon atoms comprises at least any one of an alkyl group having 1 to 6 carbon atoms and a cycloalkyl group having 3 to 10 carbon atoms.

7. The organic compound according to claim 1,
wherein n is 2 or 3.

8. The organic compound according to claim 1,
wherein each of Q¹ to Q³ represents N.

9. A light-emitting device comprising the organic compound according to claim 1.

10. A light-emitting device comprising:
a first electrode;
a second electrode; and
an EL layer positioned between the first electrode and the second electrode,
wherein the EL layer comprises a light-emitting layer and an electron-transport layer,
wherein the electron-transport layer is positioned between the light-emitting layer and the second electrode,
wherein a distance between the electron-transport layer and the second electrode is less than or equal to 5 nm, and
wherein the electron-transport layer comprises the organic compound according to claim 1.

11. An organic compound represented by General Formula (G3): 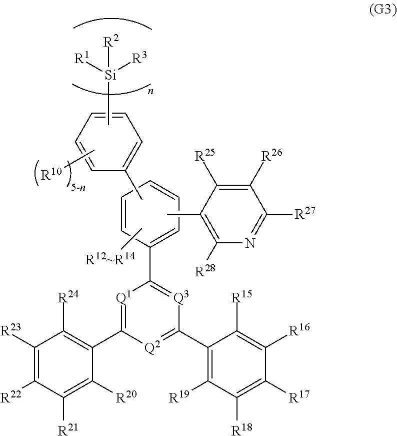
wherein:
each of Q¹ to Q³ independently represents N or CH;
at least two of Q¹ to Q³ represent N;
each of R¹ to R³ independently represents an alkyl group having 1 to 6 carbon atoms or a phenyl group;
n is an integer greater than or equal to 2 and less than or equal to 5;
R¹⁰ represents any one of hydrogen, an alkyl group having 1 to 6 carbon atoms, and a cycloalkyl group having 3 to 10 carbon atoms;
each of R¹² to R²⁴ independently represents any one of hydrogen, an alkyl group having 1 to 6 carbon atoms, a cycloalkyl group having 3 to 10 carbon atoms, and a substituted or unsubstituted aryl group having 6 to 30 carbon atoms;
each of R²⁵ to R²⁸ independently represents any one of hydrogen, an alkyl group having 1 to 6 carbon atoms, and a cycloalkyl group having 3 to 10 carbon atoms;
a plurality of R¹'s are the same or different from each other;
a plurality of R²'s are the same or different from each other;
a plurality of R³'s are the same or different from each other; and
when 5-n is greater than or equal to 2, a plurality of R¹⁰'s are the same or different from each other.

12. The organic compound according to claim 11,
wherein the organic compound is represented by General Formula (G4):

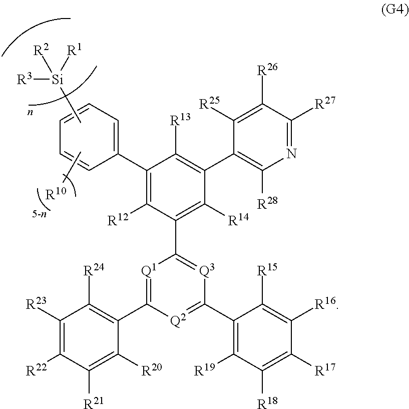

13. The organic compound according to claim 11,
wherein n is 2 or 3.

14. The organic compound according to claim 11,
wherein the organic compound is represented by General Formula (G5): 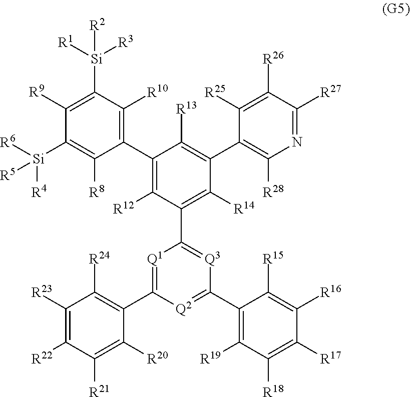
wherein:
each of R⁴ to R⁶ independently represents any one of an alkyl group having 1 to 6 carbon atoms and a phenyl group; and
each of R⁸ and R⁹ independently represents any one of hydrogen, an alkyl group having 1 to 6 carbon atoms, and a cycloalkyl group having 3 to 10 carbon atoms.

15. The organic compound according to claim 11,
wherein at least any one of R²⁵ to R²⁸ represents an alkyl group having 1 to 6 carbon atoms or a cycloalkyl group having 3 to 10 carbon atoms.

16. The organic compound according to claim 11,
wherein each of Q¹ to Q³ represents N.

17. The organic compound according to claim 11,
wherein the organic compound is represented by Structural Formula (100):

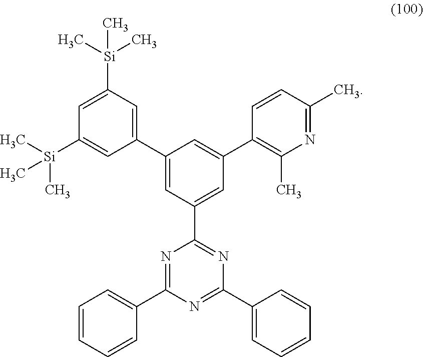 [SMILES: Cc1ccc(-c2cc(-c3cc([Si](C)(C)C)cc([Si](C)(C)C)c3)cc(-c3nc(-c4ccccc4)nc(-c4ccccc4)n3)c2)c(C)n1]

18. A light-emitting device comprising the organic compound according to claim 11.

19. A light-emitting device comprising:
a first electrode;
a second electrode; and
an EL layer positioned between the first electrode and the second electrode,
wherein the EL layer comprises a light-emitting layer and an electron-transport layer,
wherein the electron-transport layer is positioned between the light-emitting layer and the second electrode,
wherein a distance between the electron-transport layer and the second electrode is less than or equal to 5 nm, and
wherein the electron-transport layer comprises the organic compound according to claim 11.
